(19) United States
(12) Patent Application Publication
(10) Pub. No.: US 2017/0276669 A1
(43) Pub. Date: Sep. 28, 2017

(54) PRECISE ESTIMATION OF GLOMERULAR FILTRATION RATE FROM MULTIPLE BIOMARKERS

(71) Applicants: THE JOHNS HOPKINS UNIVERSITY, Baltimore, MD (US); TUFTS MEDICAL CENTER, INC., Boston, MA (US)

(72) Inventors: Josef CORESH, Pikesville, MD (US); Andrew LEVEY, Brookline, MA (US); Lesley INKER, Boston, MA (US)

(21) Appl. No.: 15/504,153

(22) PCT Filed: Aug. 11, 2015
(86) PCT No.: PCT/US2015/044567

§ 371 Date: Feb. 15, 2017

Related U.S. Application Data
(60) Provisional application No. 62/037,647, filed on Aug. 15, 2014.

(51) Int. Cl. G01N 33/53; G01N 33/68

(52) U.S. Cl. CPC G01N 33/53; G01N 33/6848; G01N 33/6851; G01N 33/6893; G01N 2800/347

(57) ABSTRACT

The present invention relates to the field of nephrology. More specifically, the present invention provides methods and compositions useful for more precisely estimating glomerular filtration rate (GFR). In a specific embodiment, a method for calculating the estimated glomerular filtration rate (eGFR) in a patient comprises the steps of (a) measuring the level of one or more metabolites using mass spectrometry from a blood sample obtained from the patient; and (b) calculating the eGFR using an algorithm that utilizes the measured levels of the one or more metabolites.

### CROSS-REFERENCE TO RELATED APPLICATIONS

This application claims the benefit of U.S. Provisional Application No. 62/037,647, filed Aug. 15, 2014, which is incorporated herein by reference in its entirety.

### STATEMENT OF GOVERNMENTAL INTEREST

This invention was made with government support under grant nos. R01DK097020, 5U01 DK067651, and 1R21 DK67651, all of which were awarded by the National Institutes of Health. The government has certain rights in the invention.

### FIELD OF THE INVENTION

The present invention relates to the field of nephrology. More specifically, the present invention provides methods and compositions useful for more precisely estimating glomerular filtration rate (GFR).

### BACKGROUND OF THE INVENTION

The diagnosis, classification, prognosis and quantification of progression of chronic kidney disease (CKD) rely heavily on estimation of glomerular filtration rate (eGFR) as a measure of kidney function. Direct measurement of GFR relying on exogenous filtration markers (mGFR) is used infrequently due to its complexity, including injection of an exogenous filtration marker. Current recommendations are therefore to use an equation including serum creatinine and covariates to estimate the GFR for most clinical and research situations. The most accurate equation for general use is the CKD Epidemiology Collaboration creatinine (CKD-EPI eGFRcr) equation published in 2009, and this is recommended by Kidney Disease International Global Outcomes (KDIGO) Guidelines for Chronic Kidney Disease. This equation has a 1-P30 of 15.9% (errors of more than 30% from the gold standard mGFR) and root mean square error of log GFR (RMSE) of 0.20, and includes demographic variables to take into account the non-GFR influences of age, sex and race on creatinine generation. Subsequent work by the CKD-EPI showed that addition of serum cystatin C to calculate eGFRcr-cys could improve precision and accuracy to 1-P30 of 8.5% in a population where CKD-EPI eGFRcr has 1-P30 of 12.8%. This demonstrated that while measures of precision and accuracy vary across populations, they can be improved by using two analytes. However, adoption of cystatin C has been slow and even this level of precision is not optimal for clinical decision making in some circumstances.

While direct GFR measurements (mGFR) are considered the gold standard, they still contain substantial imprecision. For example, in the African-American Study of Kidney Disease and Hypertension (AASK) study, two measurements of GFR using urinary clearance of I¹²⁵ Iothalamate made an average of 62 days apart had 1-P30 of 8.0%, meaning 8.0% of the measurements were outside 30% of the initial reference mGFR. In linear regression, precision of estimation is usually measured using the root mean square error (RMSE) which is the standard deviation of the residuals. In the AASK study, RMSE of a regression of the second vs. first mGFR is 0.146 on the log scale. If residuals are normally distributed, approximately 5% of the errors are outside +/−1.96*RMSE which for mGFR is +1-0.286 on the log scale (approximately +/−28.6%). Random error in mGFR does not bias regression equations to estimate GFR since regression assumes the dependent variable contains error. In contrast, estimates of the precision and accuracy with which eGFR predicts the true underlying GFR (tGFR) are inflated when mGFR has error since these estimates typically assume the gold standard is measured without error. Random error can be reduced by averaging multiple mGFRs obtaining a closer estimate of the true GFR.

Current attempts to more accurately estimate GFR remain imprecise with better estimates needed in multiple clinical setting. The need is particularly acute when current estimates are biased, such as abnormal muscle mass (e.g. wasting due to disease, amputation of a limb, obesity) or altered creatinine metabolism (e.g. creatine supplements, altered creatinine secretion in the kidney). Therefore, it is important that improved estimates be developed and validated with gold standard measured GFR, rather than surrogates such as estimated GFR by creatinine. For example, in International Application No. PCT/US/2014/037762 and U.S. Pat. No. 6,610,502, GFR was never directly measured in establishing estimated GFR. Thus, the methods described therein can only estimate “estimated” GFR. Accordingly, new methods are needed to more precisely estimate GFR.

### SUMMARY OF THE INVENTION

The present invention is based, at least in part, on the development of a panel of multiple markers based on a single blood draw to provide a precise estimate of GFR (eGFR). Current recommendations for estimating GFR call for the use of an equation that utilizes serum creatinine and covariates (age, sex, race in the most rigorously validated CKD-EPI 2009 equation). Direct measurement of GFR relying on exogenous filtration markers is used infrequently due to the requirement of several hours and collection of multiple blood or urine samples and use tracers, sometimes radioactive. The present invention provides a precise estimate of GFR (eGFR) based on multiple biomarkers in a single blood draw with excellent precision and validity in estimating GFR measured using gold standard methods which include injection of an exogenous filtration marker.

The precise estimated GFR (eGFR) is developed to estimate GFR itself (kidney function) based on gold standard GFR measurements (mGFR). Precision is enhanced by using mGFR on multiple occasions to better estimate the true underlying average GFR (tGFR). GFR estimates based on mGFR are superior to estimates based on creatinine clearance (which is biased) or GFR estimates (eGFR) based on other markers which are surrogates themselves. A table of biomarkers, with specific emphasis on metabolites, is provided each of which provides similar or better estimate of GFR than serum creatinine, the most widely used biomarker for GFR. A combination of the markers (precise panel eGFR) provides dramatically improved precision and validity compared to estimates based on serum creatinine or even cystatin C. Algorithms for combining the markers which optimize prediction are also provided and evaluated using multiple measures of precision and validity (RMSE, 1-P30, 1-P20, 1-P10, AUC, sensitivity and specificity) documenting marked improvement over the current clinical standard.

Accordingly, in one aspect, the present invention provides methods for calculating an estimated GFR (eGFR) in a patient. In a specific embodiment, a method for calculating the estimated glomerular filtration rate (eGFR) in a patient comprises the steps of (a) measuring the level of one or more metabolites using mass spectrometry from a blood sample obtained from the patient; and (b) calculating the eGFR using an algorithm that utilizes the measured levels of the one or more metabolites. In particular embodiments, the algorithm is developed using GFR measured (mGFR) using an exogenous filtration marker. Filtration markers used in mGFR include, but are not limited to, inulin, iothalamate and iohexol.

The one or more metabolites can comprise any combination of a metabolite described in Tables 2-13. In a specific embodiment, the one or more metabolites comprise one or more of X-11564. C-glycosyltryptophan, p-cresol sulfate, myo-inositol, X-02249, and pseudouridine. In another embodiment, the one or more metabolites comprise one or more of creatinine and X-11564, C-glycosyltryptophan, 1-methylhistidine, leucine, and 1-myristoylglycerophosphocholine (14:0). In yet another embodiment, the one or more metabolites comprise one or more of C-glycosyltryptophan, myo-inositol, pseudouridine, N-acetyl-1-methylhistidine, and phenylacetylglutamine.

The one or more metabolites can also comprise one or more of creatinine, C-glycosyltryptophan, pseudouridine, myo-inositol, and phenylacetylglutamine. In another embodiment, the one or more metabolites comprise one or more of X-11564, C-glycosyltryptophan, pseudouridine. X-17299, N-acetylthreonine. N-acetylserine, erythritol, arabitol, urea, and X-16394. In yet another specific embodiment, the one or more metabolites comprise one or more of X-11564, C-glycosyltryptophan, pseudouridine, X-17299, and N-acetylthreonine. In another embodiment, the one or more metabolites comprise one or more of C-glysyltryptophan*, pseudouridine. N-acetyl-threonine, N-acetylserine, and erythritol.

In particular embodiments, the one or more metabolites comprise one or more of valine, tyrosine, 4-methyl-2-oxopentanoate, glycerophosphorylcholine (GPC), uridine, threonine, X-19380, X-19411, tryptophan, X-11564, C-glycosyltryptophan*, pseudouridine, X-17299, N-acetylthreonine, N-acetylserine, erythritol, arabitol, urea, X-16394, X-11423, crythronate*, creatinine, myo-inositol, N6-carbamoylthreonyladenosine, X-12749, X-12104, N-acetylalanine, N2,N2-dimethylguanosine, 4-acetamidobutanoate, X-11945, 1-methylhistidine, arabonate, N-formylmethionine, 2-hydroxyisobutyrate, xylonate, succinylcarnitine, N-acetylneuraminate, X-12686, N-acetyl-1-methylhistidine*, homocitrulline, X-17703, X-11444, threitol, X-18887, X-12846, p-cresol sulfate, 3-methylglutarylcarnitine (C6), N-Methyl-2-pyridone-5-carboxamide, glutarylcarnitine (C5). X-16982, isobutyrylcarnitine, 3-indoxyl sulfate, X-17357, galactitol (dulcitol), X-12822, X-13837, X-02249, X-12411, X-13844, kynurenine, X-12007, X-13553, X-12125, N2,N5-diacetylornithine, O-methylcatechol sulfate, X-13835, X-12729, X-12814, leucine, and 1-myristoylglycerophosphocholine (14:0), betaine, 2-hydroxybutyrate (AHB), and X-18914.

In certain embodiments, the algorithm further utilizes serum creatinine levels. In another embodiment, the algorithm further utilizes serum cystatin C levels. The algorithm can further utilize one or more demographic parameters selected from the group consisting of age, sex and race. In a specific embodiment, the algorithm further utilizes one or more of serum creatinine levels, serum cystatin C levels, age, sex and race. In particular embodiments of the present invention, the algorithm is a linear model. In certain embodiment, the algorithm is a non-linear model.

The present invention also provides a method for calculating the estimated GFR in a patient comprising the steps of (a) measuring the level of one or more metabolites using mass spectrometry from a blood sample obtained from the patient, wherein the one or more metabolites comprise X-11564, C-glycosyltryptophan, pseudouridine, X-17299, and N-acetylthreonine; and (b) calculating the estimated GFR using an algorithm that utilizes the measured levels of the metabolites and one or more of serum creatinine levels, serum cystatin C levels, age, sex and race. In another specific embodiment, a method for calculating the estimated GFR in a patient comprises the steps of (a) measuring the level of one or more metabolites from a blood sample obtained from the patient, wherein the one or more metabolites comprise X-11564, C-glycosyltryptophan, pseudouridine, X-17299, and N-acetylthreonine; and (b) calculating the estimated GFR using an algorithm that utilizes the measured levels of the metabolites and one or more of serum creatinine levels, serum cystatin C levels, age, sex and race. The measuring step can be performed using mass spectrometry. In a specific embodiment, the measuring step is performed using high performance liquid chromatography followed by multiple reaction monitoring (MRM) mass spectrometry techniques. In particular embodiments, a cocktail of standards is added into every analyzed sample to allow for instrument performance monitoring. In another embodiment, the measuring step is performed using an immunoassay.

The present invention also provides a method for determining the estimated GFR in a patient comprising the step of calculating the estimated GFR using an algorithm that utilizes the measured levels of one or more metabolite biomarkers and one or more of serum creatinine levels, serum cystatin C levels, age, sex and race, wherein the metabolite biomarkers comprise X-11564. C-glycosyltryptophan, pseudouridine, X-17299, and N-acetylthreonine, and further wherein the metabolite biomarkers are measured from a blood sample obtained from the patient.

In particular embodiments, the algorithm is developed using GFR measured (mGFR) using an exogenous filtration marker. The algorithm can be a linear or non-linear model. In a specific embodiment, the algorithm is a stepwise regression model.

### BRIEF DESCRIPTION OF THE FIGURES

FIG. 1. Histogram of correlations with average measured GFR for 780 metabolites. Line shows the expectation under the null hypothesis.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a.” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

It is understood that when combinations, subsets, groups, etc., of these metabolite biomarkers are disclosed that while specific reference of each various individual and collective combinations and permutation of these metabolites may not be explicitly disclosed, each is specifically contemplated and described herein. For example, if a particular metabolite is disclosed, each and every possible combination of that metabolite with all the other metabolites disclosed is specifically contemplated unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application.

The present invention provides methods for precise estimation of GFR. Combinations of multiple blood analytes based on a blood draw can lead to a precise estimate of GFR (eGFR) of better precision than the current clinically used measures (cGFR using serum creatinine or even combined with serum cystatin C) and comparable (possibly better precision) than single measures of GFR (mGFR) using injection of exogenous substances. These methods can be tested in a range of clinical settings and using different measurement platforms to create new tests based on a blood measure of comparable or better precision to GFR measurements based on the gold standard clearance of exogenously injected filtration markers.

These new, more precise estimates of GFR can improve the diagnosis, classification, prognostication, risk assessement and guide to therapy for many individuals where current methods are inadequate. In addition, more precise estimates will lead to more accurate dosing of molecules (drugs and contrast agents) cleared by the kidney which can reduce subsequent toxicity and complications. These new, more precise estimates can improve precision of detecting progression of kidney disease, improving clinical care and drug development.

As described herein, a number of analytes have stronger negative correlation with kidney function than serum creatinine providing excellent use for improving the current estimates of kidney function (pseudouridine, N-acetylthreonine, N-acetylserine, erythritol, arabitol and erythronate; metabolites measureable but only known by their precise mass spectrographic characteristics but unnamed: X-11564, X-17299, X-16394, X-11423; metabolites known to be associated with kidney function but precision was uncertain: C-glycosyltryptophan; metabolites often used in estimating GFR: creatinine and urea).

A number of analytes have a strong positive correlation with kidney function. They can be used to improve detection deficiencies and adverse metabolic alterations when kidney function is low (strongest correlates include valine, tyrosine, 4-methyl-2-oxopentanoate, glycerophosphorylcholine (GPC), uridine, threonine and tryptophan; metabolites measureable but only known by their precise mass spectrographic characteristics but unnamed: X-19380, X-19411; less strongly correlated but selected by stepwise regression as useful in improving eGFR are: leucine, 1-myristoylglycerophosphocholine (14:0)).

As further described herein, different algorithms can be used to combine the markers, all of which improve on the current clinical standard eGFRcr. This allows for flexibility which can reduce susceptibility to error when specific factors influencing any one metabolite are present (e.g., reduced muscle mass leading to eGFRcr which is biased towards high values missing cases of kidney disease or its progression). eGFR can be calculated using a one-step algorithm or individual estimates from each metabolite, or group of metabolites, and then these can be combined using robust methods which average while down weighting outlier values which may be unreliable in the individual.

### I. Definitions

The terms “patient,” “individual,” or “subject” are used interchangeably herein, and refer to a mammal, particularly, a human. The patient may have a mild, intermediate or severe disease or condition. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

The terms “measuring” and “determining” are used interchangeably throughout, and refer to methods which include obtaining or providing a patient sample and/or detecting the level of a metabolite biomarker(s) in a sample. In one embodiment, the terms refer to obtaining or providing a patient sample and detecting the level of one or more metabolite biomarkers in the sample. In another embodiment, the terms “measuring” and “determining” mean detecting the level of one or more metabolite biomarkers in a patient sample. The term “measuring” is also used interchangeably throughout with the term “detecting.” In certain embodiments, the term is also used interchangeably with the term “quantitating.”

The terms “sample,” “patient sample,” “biological sample,” and the like, encompass a variety of sample types obtained from a patient, individual, or subject and can be used in a diagnostic or monitoring assay. In particular embodiments, the patient sample may be obtained from a healthy subject, a diseased patient or a patient having associated symptoms of CKD. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In a specific embodiment, a sample comprises a blood sample. In another embodiment, a sample comprises a plasma sample. In yet another embodiment, a serum sample is used.

The definition of “sample” can also include, in certain embodiments, samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any functional, antigen-binding fragment or derivative of any of the herein described antibodies.

As used herein, the term “antigen” is generally used in reference to any substance that is capable of reacting with an antibody. More specifically, as used herein, the term “antigen” refers to a metabolite described herein. An antigen can also refer to a synthetic peptide, polypeptide, protein or fragment of a polypeptide or protein, or other molecule which elicits an antibody response in a subject, or is recognized and bound by an antibody.

As used herein, the term “biomarker” refers to a molecule that is associated either quantitatively or qualitatively with a biological change. Examples of biomarkers include metabolites, polypeptides, proteins or fragments of a polypeptide or protein; and polynucleotides, such as a gene product, RNA or RNA fragment. In certain embodiments, a “biomarker” means a compound that is differentially present (i.e., increased or decreased) in a biological sample from a subject or a group of subjects having a first phenotype (e.g., having a disease or condition) as compared to a biological sample from a subject or group of subjects having a second phenotype (e.g., not having the disease or condition or having a less severe version of the disease or condition). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%/0, by at least 130%, by at least 140%/0, by at least 150%, or more, or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using, for example, either Welch's T-test or Wilcoxon's rank-sum Test). Biomarker levels can be used, in conjunction with other parameters (e.g., creatinine, cystatin and/or other demographics (e.g., age, race, sex)) to calculate estimated GFR in a patient.

In certain embodiments, the terms “comparing” or “comparison” can refer to making an assessment of how the level or proportion of one or more biomarkers in a sample from a patient relates to the level or proportion of the corresponding one or more biomarkers in a standard or control sample. For example, “comparing” may refer to assessing whether the level or proportion of one or more biomarkers in a sample from a patient is the same as, more or less than, or different from the level or proportion of the corresponding one or more biomarkers in standard or control sample. More specifically, the term may refer to assessing whether the level or proportion of one or more biomarkers in a sample from a patient is the same as, more or less than, different from or otherwise corresponds (or not) to the level or proportion of predefined biomarker levels/ratios that correspond to a particular disease, disorder or condition. In another embodiment, the terms “comparing” or “comparison” refers to making an assessment of how the level or proportion of one or more biomarkers in a sample from a patient relates to the level or proportion of another biomarker in the same sample. For example, a ratio of one biomarker to another from the same patient sample can be compared. Ratios of metabolite biomarkers can be compared to other ratios in the same sample or to predefined reference or control ratios.

As used herein, the terms “indicates” or “correlates” (or “indicating” or “correlating.” or “indication” or “correlation,” depending on the context) can mean that the patient has a particular eGFR. In specific embodiments, a particular set or pattern of the amounts of one or more metabolite biomarkers (and other parameters (e.g., creatinine, cystatin and/or other demographics (e.g., age, race, sex)) may be correlated to an estimated GFR. In certain embodiments. “indicating.” or “correlating.” as used according to the present invention, may comprise any linear or non-linear method of quantifying the relationship among levels/ratios of biomarkers and other parameters (e.g., creatinine, cystatin, and/or demographics) for the estimation of GFR.

Various methodologies of the instant invention can include a step that involves comparing a value, level, feature, characteristic, property, etc. to a “suitable control,” referred to interchangeably herein as an “appropriate control,” a “control sample,” a “reference” or simply a “control.” A “suitable control,” “appropriate control,” “control sample,” “reference” or a “control” is any control or standard familiar to one of ordinary skill in the art useful for comparison purposes. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition. “reference levels” of combinations of biomarkers may also be ratios of absolute or relative amounts or concentrations of two or more biomarkers with respect to each other. Such reference levels may also be tailored to specific techniques that are used to measure levels of biomarkers in biological samples (e.g., LC-MS, GC-MS. ELISA, PCR, etc.), where the levels of biomarkers may differ based on the specific technique that is used.

As used herein, the term “predetermined threshold value” of a biomarker refers to the level of the same biomarker in a corresponding control/normal sample or group of control/normal samples obtained from normal, or healthy, subjects, e.g., subjects who do not have a kidney disease, disorder or condition. Further, the term “altered level” of a biomarker in a sample refers to a level that is either below or above the predetermined threshold value for the same biomarker and thus encompasses either high (increased) or low (decreased) levels.

The terms “specifically binds to.” “specific for,” and related grammatical variants refer to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly. “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M. As used herein, the terms “specific binding” or “specifically binding” when used in reference to the interaction of an antibody and a protein or peptide means that the interaction is dependent upon the presence of a particular structure (i.e., the epitope) on the protein.

As used herein, the terms “binding agent specific for” or “binding agent that specifically binds” refers to an agent that binds to a biomarker and does not significantly bind to unrelated compounds. Examples of binding agents that can be effectively employed in the disclosed methods include, but are not limited to, proteins and antibodies, such as monoclonal or polyclonal antibodies, or antigen-binding fragments thereof, aptamers, lectins, etc. In certain embodiments, a binding agent binds a biomarker (e.g., a metabolite biomarker) with an affinity constant of, for example, greater than or equal to about 1×10⁻⁶ M.

### II. Detection of GFR Metabolite Biomarkers

A. Detection by Mass Spectrometry

In one aspect, the metabolite biomarkers of the present invention may be detected by mass spectrometry, a method that employs a mass spectrometer to detect gas phase ions. Examples of mass spectrometers are time-of-flight, magnetic sector, quadrupole filter, ion trap, ion cyclotron resonance, Orbitrap, hybrids or combinations of the foregoing, and the like.

In particular embodiments, the biomarkers of the present invention are detected using selected reaction monitoring (SRM) mass spectrometry techniques. Selected reaction monitoring (SRM) is a non-scanning mass spectrometry technique, performed on triple quadrupole-like instruments and in which collision-induced dissociation is used as a means to increase selectivity. In SRM experiments two mass analyzers are used as static mass filters, to monitor a particular fragment ion of a selected precursor ion. The specific pair of mass-over-charge (m/z) values associated to the precursor and fragment ions selected is referred to as a “transition” and can be written as parent m/z→fragment m/z (e.g. 673.5→534.3). Unlike common MS based proteomics, no mass spectra are recorded in a SRM analysis. Instead, the detector acts as counting device for the ions matching the selected transition thereby returning an intensity distribution over time. Multiple SRM transitions can be measured within the same experiment on the chromatographic time scale by rapidly toggling between the different precursor/fragment pairs (sometimes called multiple reaction monitoring, MRM). Typically, the triple quadrupole instrument cycles through a series of transitions and records the signal of each transition as a function of the elution time. The method allows for additional selectivity by monitoring the chromatographic coelution of multiple transitions for a given analyte. The terms SRM/MRM are occasionally used also to describe experiments conducted in mass spectrometers other than triple quadrupoles (e.g. in trapping instruments) where upon fragmentation of a specific precursor ion a narrow mass range is scanned in MS2 mode, centered on a fragment ion specific to the precursor of interest or in general in experiments where fragmentation in the collision cell is used as a means to increase selectivity. In this application the terms SRM and MRM or also SRM/MRM can be used interchangeably, since they both refer to the same mass spectrometer operating principle. As a matter of clarity, the term MRM is used throughout the text, but the term includes both SRM and MRM, as well as any analogous technique, such as e.g. highly-selective reaction monitoring, hSRM, LC-SRM or any other SRM/MRM-like or SRM/MRM-mimicking approaches performed on any type of mass spectrometer and/or, in which the peptides are fragmented using any other fragmentation method such as e.g. CAD (collision-activated dissociation (also known as CID or collision-induced dissociation), HCD (higher energy CID), ECD (electron capture dissociation), PD (photodissociation) or ETD (electron transfer dissociation).

In another specific embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. For example, MALDI-TOF can be utilized with trypsin digestion and tandem mass spectrometry as described herein.

In an alternative embodiment, the mass spectrometric technique comprises surface enhanced laser desorption and ionization or “SELDI,” as described, for example, in U.S. Pat. No. 6,225,047 and U.S. Pat. No. 5,719,060. Briefly, SELDI refers to a method of desorption/ionization gas phase ion spectrometry (e.g. mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes comprising energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker panel, pursuant to the present invention.

In another mass spectrometry method, the biomarkers can be first captured on a chromatographic resin having chromatographic properties that bind the biomarkers. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

B. Detection by Immunoassay

In other embodiments, the metabolite biomarkers of the present invention can be detected and/or measured by immunoassay. Immunoassay requires specific capture reagents/binding agent, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

In certain embodiments, the levels of the metabolite biomarkers employed herein are quantified by immunoassay, such as enzyme-linked immunoassay (ELISA) technology. In specific embodiments, the levels of expression of the biomarkers are determined by contacting the biological sample with antibodies, or antigen binding fragments thereof, that selectively bind to the metabolite biomarkers; and detecting binding of the antibodies, or antigen binding fragments thereof, to the metabolite biomarkers. In certain embodiments, the binding agents employed in the disclosed methods and compositions are labeled with a detectable moiety.

For example, the level of a metabolite biomarker in a sample can be assayed by contacting the biological sample with an antibody, or antigen binding fragment thereof, that selectively binds to the target biomarker (referred to as a capture molecule or antibody or a binding agent), and detecting the binding of the antibody, or antigen-binding fragment thereof, to the biomarker. The detection can be performed using a second antibody to bind to the capture antibody complexed with its target metabolite biomarker. Kits for the detection of biomarkers as described herein can include pre-coated strip plates, biotinylated secondary antibody, standards, controls, buffers, streptavidin-horse radish peroxidise (HRP), tetramethyl benzidine (TMB), stop reagents, and detailed instructions for carrying out the tests including performing standards.

The present disclosure also provides methods in which the levels of the metabolite biomarkers in a biological sample are determined simultaneously. For example, in one embodiment, methods are provided that comprise: (a) contacting a biological sample obtained from the subject with a plurality of binding agents that selectively bind to a plurality of metabolite biomarkers disclosed herein for a period of time sufficient to form binding agent-biomarker complexes: (b) detecting binding of the binding agents to the plurality of metabolite biomarkers, thereby determining the levels of the metabolite biomarkers in the biological sample; and (c) comparing the levels of the plurality of metabolite biomarkers in the biological sample with predetermined threshold values, wherein levels of at least one of the plurality of metabolite biomarkers above/below the predetermined threshold values can be used to calculate eGFR. Examples of binding agents that can be effectively employed in such methods include, but are not limited to, antibodies or antigen-binding fragments thereof, aptamers, lectins and the like.

In a further aspect, the present disclosure provides compositions that can be employed in the disclosed methods. In certain embodiments, such compositions a solid substrate and a plurality of binding agents immobilized on the substrate, wherein each of the binding agents is immobilized at a different, indexable, location on the substrate and the binding agents selectively bind to a plurality of metabolite biomarkers disclosed herein. In a specific embodiment, the locations are pre-determined. In other embodiments, kits are provided that comprise such compositions. In certain embodiments, the plurality of metabolite biomarkers includes one or more of the metabolites described herein including X-11564, C-glycosyltryptophan, pseudouridine, X-17299, N-acetylthreonine, N-acetylserine, erythritol, arabitol, urea, and X-16394. In other embodiments, the plurality of metabolite biomarkers further includes at least one metabolite biomarker selected from the group consisting of valine, tyrosine, 4-methyl-2-oxopentanoate, glycerophosphorylcholine (GPC), uridine, threonine, X-19380, X-19411, and tryptophan. The plurality of metabolite biomarkers can comprise X-11564, C-glycosyltryptophan, pseudouridine, X-17299, and N-acetylthreonine. In other embodiments, the plurality of metabolite biomarkers comprises C-glysyltryptophan*, pseudouridine, N-acetyl-threonine, N-acetylserine, and erythritol. In general, the plurality of metabolite biomarkers can comprise one or more of valine, tyrosine, 4-methyl-2-oxopentanoate, glycerophosphorylcholine (GPC), uridine, threonine. X-19380, X-19411, tryptophan, X-11564, C-glycosyltryptophan*, pseudouridine, X-17299, N-acetylthreonine, N-acetylserine, erythritol, arabitol, urea, X-16394. X-11423, erythronate*, creatinine, myo-inositol. N6-carbamoylthreonyladenosine. X-12749, X-12104, N-acetylalanine, N2,N2-dimethylguanosine, 4-acetamidobutanoate, X-11945, 1-methylhistidine, arabonate, N-formylmethionine, 2-hydroxyisobutyrate, xylonate, succinylcarnitine, N-acetylneuraminate, X-12686, N-acetyl-1-methylhistidine*, homocitrulline, X-17703, X-11444, threitol, X-18887, X-12846, p-cresol sulfate, 3-methylglutarylcarnitine (C6), N1-Methyl-2-pyridone-5-carboxamide, glutarylcarnitine (C5), X-16982, isobutyrylcarnitine, 3-indoxyl sulfate, X-17357, galactitol (dulcitol), X-12822, X-13837, X-02249, X-12411, X-13844, kynurenine, X-12007, X-13553, X-12125, N2,N5-diacetylornithine, O-methylcatechol sulfate, X-13835, X-12729, X-12814, leucine, and 1-myristoylglycerophosphocholine (14:0), betaine, 2-hydroxybutyrate (AHB), X-18914. In other embodiments, such compositions additionally comprise binding agents that selectively bind to other biomarkers. Binding agents that can be employed in such compositions include, but are not limited to, antibodies, or antigen-binding fragments thereof, aptamers, lectins, other metabolites and the like.

In a related aspect, methods for calculating eGFR in a subject are provided, such methods comprising: (a) contacting a biological sample obtained from the subject with a composition disclosed herein for a period of time sufficient to form binding agent-metabolite biomarker complexes; (b) detecting binding of the binding agents to a plurality of metabolite biomarkers, thereby determining the levels of metabolite biomarkers in the biological sample; and (c) comparing the levels of metabolite biomarkers in the biological sample with predetermined threshold values, wherein levels of expression of at least one of the plurality of metabolite biomarkers above/below the predetermined threshold values can be used to calculate eGFR.

Although antibodies are useful because of their extensive characterization, any other suitable agent (e.g., a peptide, an aptamer, or a small organic molecule) that specifically binds a metabolite biomarker of the present invention is optionally used in place of the antibody in the above described immunoassays. For example, an aptamer that specifically binds a metabolite biomarker and/or one or more of its further breakdown products might be used. Aptamers are nucleic acid-based molecules that bind specific ligands. Methods for making aptamers with a particular binding specificity are known as detailed in U.S. Pat. No. 5,475,096; U.S. Pat. No. 5,670,637; U.S. Pat. No. 5,696,249; U.S. Pat. No. 5,270,163; U.S. Pat. No. 5,707,796; U.S. Pat. No. 5,595,877; U.S. Pat. No. 5,660,985; U.S. Pat. No. 5,567,588; U.S. Pat. No. 5,683,867; U.S. Pat. No. 5,637,459; and U.S. Pat. No. 6,011,020.

In specific embodiments, the assay performed on the biological sample can comprise contacting the biological sample with one or more capture agents (e.g., antibodies, peptides, aptamer, etc., combinations thereof) to form a metabolite biomarker:capture agent complex. The complexes can then be detected and/or quantified.

In one method, a first, or capture, binding agent, such as an antibody that specifically binds the metabolite biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker is then used to detect binding of the metabolite biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In another embodiment, the assay is a competitive binding assay, wherein labeled biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, chips and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different metabolite biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, are well known in the art. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis. In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

C. Other Methods for Detecting Metabolite Biomarkers

In several embodiments, the metabolite biomarkers of the present invention may be detected by means of an electrochemicaluminescent assay, for example, developed by Meso Scale Discovery (Gaithersrburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. No. 7,497,997; U.S. Pat. No. 7,491,540; U.S. Pat. No. 7,288,410; U.S. Pat. No. 7,036,946; U.S. Pat. No. 7,052,861; U.S. Pat. No. 6,977,722; U.S. Pat. No. 6,919,173; U.S. Pat. No. 6,673,533; U.S. Pat. No. 6,413,783; U.S. Pat. No. 6,362,011; U.S. Pat. No. 6,319,670; U.S. Pat. No. 6,207,369; U.S. Pat. No. 6,140,045; U.S. Pat. No. 6,090,545; and U.S. Pat. No. 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

The metabolite biomarkers of the present invention can also be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry). Furthermore, a sample may also be analyzed by means of a chip. Chips generally comprise solid substrates and have a generally planar surface, to which a capture reagent (also called an adsorbent or affinity reagent) is attached. Frequently, the surface of a chip comprises a plurality of addressable locations, each of which has the capture reagent bound there. These include, for example, chips produced by Advion, Inc. (Ithaca. N.Y.).

### II. Determination of a Patient's Glomerular Filtration Rate Status

A. Metabolite Biomarker Panels

The present invention relates to the use of metabolite biomarkers to calculate an estimated GFR. A patient's eGFR can be calculated using one or more metabolite biomarkers described herein, serum creatinine, serum cystatin C, and/or demographics. More specifically, the biomarkers of the present invention include a metabolite described herein including any combinations of metabolites listed in Tables 2-13. In particular embodiments, the biomarkers of the present invention include, but are not limited to, valine, tyrosine, 4-methyl-2-oxopentanoate, glycerophosphorylcholine (GPC), uridine, threonine, X-19380, X-19411, tryptophan, X-11564, C-glycosyltryptophan*, pseudouridine, X-17299, N-acetylthreonine, N-acetylserine, erythritol, arabitol, urea, X-16394, X-11423, erythronate*, creatinine, myo-inositol, N6-carbamoylthreonyladenosine, X-12749, X-12104, N-acetylalanine, N2,N2-dimethylguanosine, 4-acetamidobutanoate, X-11945, 1-methylhistidine, arabonate, N-formylmethionine, 2-hydroxyisobutyrate, xylonate, succinylcarnitine, N-acetylneuraminate, X-12686, N-acetyl-1-methylhistidine*, homocitrulline. X-17703, X-11444, threitol, X-18887, X-12846, p-cresol sulfate, 3-methylglutarylcarnitine (C6), N1-Methyl-2-pyridone-5-carboxamide, glutarylcarnitine (C5), X-16982, isobutyrylcarnitine, 3-indoxyl sulfate, X-17357, galactitol (dulcitol), X-12822, X-13837. X-02249. X-12411, X-13844, kynurenine, X-12007, X-13553, X-12125. N2,N5-diacetylornithine, O-methylcatechol sulfate, X-13835, X-12729, X-12814, leucine and 1-myristoylglycerophosphocholine (14:0), betaine, 2-hydroxybutyrate (AHB), and X-18914. Other biomarkers known in the relevant art may be used in combination with the biomarkers described herein.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the biomarker panels of the present invention may show a statistical difference in different GFR statuses of at least p<0.05, p<10⁻², p<10⁻³, p<10⁻⁴ or p<10⁻⁵. Diagnostic tests that use these biomarkers may show an ROC of at least 0.6, at least about 0.7, at least about 0.8, or at least about 0.9.

Furthermore, in certain embodiments, the values measured for markers of a biomarker panel are mathematically combined and the combined value is correlated to the underlying diagnostic question. Biomarker values may be combined by any appropriate state of the art mathematical method. Well-known mathematical methods for correlating a marker combination to a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART. Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in a correlating a biomarker combination of the present invention, e.g. to determine/calculate GFR, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone. C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

B. Generation of Classification Algorithms for Qualifying GFR Status

In some embodiments, data that are generated using samples such as “known samples” can then be used to “train” a classification model. A “known sample” is a sample that has been pre-classified. The data that are used to form the classification model can be referred to as a “training data set.” The training data set that is used to form the classification model may comprise raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g., Bayesian classifier or Fischer analysis), logistic classifiers, and support vector classifiers (support vector machines).

Another supervised classification method is a recursive partitioning process. Recursive partitioning processes use recursive partitioning trees to classify data derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application Publication No. 2002/0193950 (Gavin et al. “Method or analyzing mass spectra”). U.S. Patent Application Publication No. 2003/0004402 (Hitt et al., “Process for discriminating between biological states based on hidden patterns from biological data”), and U.S. Patent Application Publication No. 2003/0055615 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows® or Linux™ based operating system. In embodiments utilizing a mass spectrometer, the digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including R, C. C++, visual basic, etc.

The learning algorithms described above are useful both for developing classification algorithms for the biomarkers already discovered, and for finding new biomarker biomarkers. The classification algorithms, in turn, form the base for diagnostic tests by providing diagnostic values (e.g., cut-off points) for biomarkers used singly or in combination.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Precise Estimation of GFR from Multiple Blood Biomarkers

### Materials and Methods

Study Population.

Metabolite discovery used stored serum from 200 individuals with GFR measurements using urinary clearance of 1-125 Iothalamate in the African-American Study of Kidney Disease and Hypertension (AASK) at the 48 month follow-up visit. This subset selected as having reliable mGFRs by choosing individuals whose mGFR at the 42 and 54 months follow-up visits were within 25% of the mGFR at the 48 month visit.

GFR Measurement.

GFR was measured as the weighted mean of 4 timed voluntary ¹²⁵I-iothalamate urinary clearances of 25-35 minutes' duration. Comparisons of ¹²⁵I-iothalamate clearances to urinary clearance of inulin, the reference standard for GFR measurements, showed high correlations.

Clinical Chemistry Measurements.

SCr was assayed using the Beckman rate-Jaffé method based on the alkaline picrate reaction (reference range, 0.8-1.4 mg/dL) and calibrated to standardized SCr values measured at the Cleveland Clinic Research Laboratory subsequently calibrate to IDMS traceable methods. Results of the calibration procedure have been described previously. Stevens et al., 57(3 Suppl. 2) AM. J. KIDNEY DIS. S9-16 (2011); Stevens et al., 50(1) AM. J. KIDNEY DIS. 23-35 (2007).

To measure SCysC, stored serum specimens were thawed in 2005-2006 after being frozen at −70° C. since collection. Samples were assayed at the Cleveland Clinic Research Laboratory using a particle-enhanced immunonephelometric assay (N Latex Cystatin C; Dade Behring) of 0.97 and 1.90 mg/L (72.7 and 142.3 mol/L), respectively. SCysC has been shown to be robust to multiple freeze-thaw cycles.

Metabolomic Measurements.

Metabolite profiling was measured using serum samples collected during the AASK study and frozen at −80° C. Detection and quantification of 829 metabolites was completed by Metabolon Inc. (Durham, USA) using an untargeted, gas chromatography-mass spectrometry and liquid chromatography-mass spectrometry (GC-MS and LC-MS)-based metabolomic quantification protocol. Evans et al., 81(16) ANAL. CHEM. 6656-67 (2009); Ohta et al., 37(4) TOXIOCOLOIC PATH. 521-35 (2009). Values were standardized for each metabolite and 49 metabolites with no variation (all values 1.0) were excluded leaving 780 metabolites.

Sample Preparation and Metabolic Profiling: The non-targeted metabolic profiling platform employed for this analysis combined three independent platforms implemented by Metabolon under a service agreement using these methods: ultrahigh performance liquid chromatography/tandem mass spectrometry (UHPLC/MS/MS) optimized for basic species. UHPLC/MS/MS optimized for acidic species, and gas chromatography/mass spectrometry (GC/MS). Samples were processed essentially as described previously (Ohta T, Masutomi N, Tsutsui, N, et al. Untargeted metabolomic profiling as an evaluative tool of fenofibrate-induced toxicology in Fischer 344 male rats. Toxicol. Pathol. 2009; 37(4)521; Evans A M, DeHaven C D, Barrett T, Mitchell M, and Milgram E. Integrated, nontargeted ultrahigh performance liquid chromatography/electrospray ionization tandem mass spectrometry platform for the identification and relative quantification of the small-molecule complement of biological systems. Anal. Chem. 2009; 81:6656-67). For each sample, 100 μL of serum was used for analyses. Using an automated liquid handler (Hamilton LabStar, Salt Lake City, Utah), protein was precipitated with methanol that contained four standards to report on extraction efficiency. The resulting supernatant was split into equal aliquots for analysis on the three platforms. Aliquots, dried under nitrogen and vacuum-desiccated, were subsequently either reconstituted in 50 μL 0.1% formic acid in water (acidic conditions) or in 50 μL 6.5 mM ammonium bicarbonate in water, pH 8 (basic conditions) for the two UHPLC/MS/MS analyses or derivatized to a final volume of 50 μL for GC/MS analysis using equal parts bistrimethyl-silyl-trifluoroacetamide and solvent mixture acetonitrile:dichloromethane:cyclohexane (5:4:1) with 5% triethylamine at 60° C. for one hour. In addition, three types of controls were analyzed in concert with the experimental samples: aliquots of a “client matrix” formed by pooling a small amount of each sample served as technical replicates throughout the data set, extracted water samples served as process blanks, and a cocktail of standards spiked into every analyzed sample allowed instrument performance monitoring. Experimental samples and controls were randomized across six platform run days.

For UHLC/MS/MS analysis, aliquots were separated using a Waters Acquity UPLC (Waters, Millford, Mass.) and analyzed using an LTQ mass spectrometer (Thermo Fisher Scientific, Inc., Waltham, Mass.) which consisted of an electrospray ionization (ESI) source and linear ion-trap (LIT) mass analyzer. The MS instrument scanned 99-1000 m/z and alternated between MS and MS² scans using dynamic exclusion with approximately 6 scans per second. Derivatized samples for GC/MS were separated on a 5% phenyldimethyl silicone column with helium as the carrier gas and a temperature ramp from 60° C. to 340° C. and then analyzed on a Thermo-Finnigan Trace DSQ MS (Thermo Fisher Scientific, Inc.) operated at unit mass resolving power with electron impact ionization and a 50-750 atomic mass unit scan range.

Metabolites were identified by automated comparison of the ion features in the experimental samples to a reference library of chemical standard entries that included retention time, molecular weight (m/z), preferred adducts, and in-source fragments as well as associated MS spectra, and were curated by visual inspection for quality control using software developed at Metabolon (DeHaven C D. Evans A M, Dai H, and Lawton K A. Organization of GC/MS and LC/MS Metabolomics data into Chemical Libraries. J. Cheminform. 2010; 2(1):9).

For data display purposes and statistical analysis, each biochemical was rescaled to set the median equal to 1. In addition, any missing values were assumed to be below the limits of detection and these values were imputed with the compound minimum (minimum value imputation).

Data Analysis.

GFR was averaged across the 3 consistent mGFRs (measured at 42, 48 and 54 months) to provide the most precise estimate of true GFR which is the primary outcomes to be estimated in this study, referred to as MGFR (log of the average of 3 consistent mGFRs). GFR and metabolites were log transformed to allow for the physiologically expected inverse association between GFR and filtration markers.

Correlations were calculated between all 780 metabolites and MGFR. Metabolites with correlations of similar or greater negative values to log of serum creatinine (Scr) were considered the most promising. Combinations of metabolites were then examined for their predictive ability for producing a precise estimated GFR (eGFR). In particular embodiments, non-linear algorithms that emphasize consensus estimates and exclude outliers are used for robustness. In other embodiments, linear regression algorithms can be used. Because linear regression was sufficient to show superiority to the currently used algorithms, the following discussion focuses on multiple linear regression.

Combinations of metabolites were explored in several groupings of specific clinical utility: (1) Metabolites only excluding demographic covariates since this would simplify GFR estimation and may prove to be more robust to patient characteristics; (2) Metabolites with demographics; (3) Known metabolites; and (4) Above with traditional markers (log serum creatinine and cystatin C).

Predictions were compared to the gold standard MGFR for different measures of precision and validity: (1) RMSE-root mean square error providing a continuous measure of precision: and (2) 1-P30, 1-P20 and 1-P10 which estimate the percentage of estimates which are further than 30%, 20%, and 10% of the gold standard. These estimates were compared across models using bootstrapping.

The current clinical standards of the CKD-EPI equation that uses serum creatinine and demographics for estimating GFR was used as the main comparison with the goal of showing superiority. We also compared this result to a best fit equation with creatinine and demographics fit in this dataset. We use the dedicated method to assay creatinine, the Jaffe assay, in routine clinical chemistry as the primary comparison but also show the performance of the less precise metabolite discovery creatinine assay. We recognize that mass spectrography (MS) can be optimized to yield creatinine measurements with similar precision and greater validity than the Jaffe assay, while the current MS creatinine discovery assay had lower precision. In addition, cystatin C and the combination of creatinine and cystatin C were examined as proposed estimates which have been rigorously examined but are much less widely used.

### Results

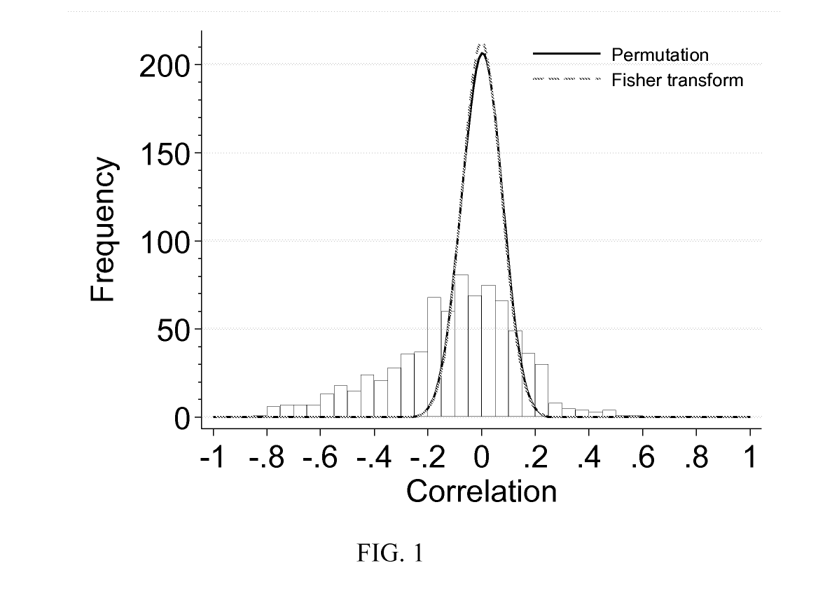

Twelve participants had missing serum creatinine Jaffe data and were excluded from the analysis. The baseline characteristics of the study participants (Table 1) were similar to those of the overall AASK study. Mean MGFR was 48 (range 10-94) ml/min/1.73 m.² The correlations of metabolites with the MGFR was centered around zero with an excess of metabolites with a strong negative correlation (FIG. 1). A dozen markers showed a stronger correlation than serum creatinine (identified M513 in the Metabolon panel) with another dozen analytes having weaker correlation than creatinine but still lower than −0.60. Table 13 shows a list of all metabolites ranked by their correlation with MGFR, including 9 metabolites with strong positive correlations (>0.40, p<0.001). Random permutation of the MGFR shows that if the null hypothesis were true then 95%, 99% and minimum-maximum of the correlations with marker values would be in these intervals −0.14 to 0.14, −0.18 to 0.18 and −0.22 to 0.21 (average of 500 simulations).

Performance of serum creatinine improves when measured using the Jaffe clinical chemistry assay compared to its measurement as part of the discovery panel (RMSE declines from 0.29 to 0.23 without demographics). As expected, serum creatinine based estimates are much better when age and sex are included in the regression models (RMSE 0.26 for Metabolon screen and 0.19 for Jaffe creatinine). eGFRcr using the clinically accepted CKD-EPI equation performs very similarly to a regression optimized for the AASK study in this sample (RMSE 0.201 vs. 0.191) suggesting we can use it as a reference representing both the current clinical practice and the best creatinine performance when combined with demographics.

In models without demographics each of the top 10 markers results in more precise estimates (higher correlation and lower RMSE) than serum creatinine measured using the Metabolomic discovery method with 3 of the metabolites (X-11564, C-glycosyltryptophan and pseudouridine) having stronger correlations than even serum creatinine assayed using the Jaffe assay. The combination of top 5 metabolites improves the RMSE to 0.1448 (1-P30 of 3.19%) and this is significantly better than the precision obtained by the clinically accepted CKD-EPI eGFRcr (RMSE 0.2008, 1-P30 7.98%, p=0.04). The prediction by the top 5 and top 10 metabolite improves only modestly with incorporation of demographic variables suggesting they are not strongly related to age and sex (Table 13 shows correlation of markers with age and sex). Sensitivity analyses show that panels with good precision and low error rates can be constructed even if unnamed metabolites are excluded (Table 5, RMSE 0.1577 and 0.1483 for top 5 and top 10 known metabolites with corresponding 1-P30 or 3.19% and 1.60%).

In this dataset, RMSE and 1-P30 is 0.170 and 4.8% and 0.140 and 4.3% for CKD-EPIcr-cys and regression with log creatinine, log cystatin and metabolites, respectively. When the top 5 metabolites are combined with these four variables, the RMSE declines to 0.1279 and 1-P30 reduces to 1.06% i (p=0.008).

Stepwise regression as well as other algorithms allow for more parsimonious selection of subsets of analytes that yield excellent improved precision. For all metabolites and limited to those with known names respectively, Tables 4 and 5 list performance of these models and Tables 11 and 12 list the specific analytes and regression coefficients. Models were also constructed that specifically included the Jaffe creatinine assay since some high precision method to estimate creatinine may be desirable to include in a panel precisely estimating GFR. Likewise, models which include demographics are explored. Overall, a number of models can yield excellent precision and show improved statistical significance compared to eGFRcr. For example, the best stepwise model considering creatinine has RMSE of 0.144 with 4 known analytes (C-glycosyltryptophan, pseudouridine, myo-inositol, phenylacetylglutamine) improving the percentage of large errors (1-P30) to 1.6% from 8% (p<0.01) for eGFRcr (1-P20 improved to 16.5% from 25.0%, p<0.05). Considering unknown analytes and/or cystatin C can provide similar or even somewhat better precision showing a range of options for excellent precision in estimating measured GFR (Table 4, 5, 11 and 12). It is also noteworthy that in some models, metabolites positively correlated with GFR, improve the estimates; the most useful among these were leucine and 1-myristoylglycerophosphocholine (14:0).

### Discussion

An unbiased metabolomics screen revealed many metabolites that are strongly negatively correlated with measured GFR. Combining metabolites into a panel to precisely estimate GFR (precise eGFR) resulted in extremely precise estimates which were clearly superior to the currently used eGFRcr, even without the use of demographics or creatinine itself. These panels were more precise than estimates using the low molecular weight protein, cystatin C. Multiple panels and algorithms perform well which can be useful in adapting to a wide range of clinical situations. Adding cystatin C to creatinine, demographics and other top metabolites resulted in the most precise eGFR which nearly eliminated large errors (1-P30 1.1% vs. 8.0%/o with eGFRcr, 6.9%/o for eGFRcys and 4.8% for eGFRcr-cys). These levels of precision are as good or better than that seen with single measures of GFR.

The previous literature on metabolites related to kidney function focused on using eGFRcr as the gold standard. Several previous papers show correlations between metabolites and eGFRcr which is useful but the previous approaches do not lead to a fully enabled concept since merely being a measure of kidney function which is equivalent to creatinine is not useful. To be clinically useful, the test must be superior to the existing clinical standard (eGFRcr) and the promising new estimates (eGFRcys and eGFRcr-cys). The current approach of using measured GFR allows for an unbiased comparison to these clinical standards and provides clear evidence of several analytes and algorithms results in statistically significant improvement. Showing the relationship of metabolites to prognosis is of utility as well and several papers have shown associations with incidence of CKD association with CKD stage some with emphasis on cGFRcr, uremia, risk of CKD progression and ESRD. Some found no added value in improving the correlation with eGFR (association of metabolites with diet).

The present study has several strengths and limitations. The strengths include use of a gold standard measure of GFR in a study (AASK) which contributed to development of the MDRD Study and CKD-EPI eGFR equations. The gold standard's precision is enhanced by focusing the average of three successive GFR measures in a sample in which all three measures are consistent with the middle measure so that we have a very high level of confidence in the fold standard minimizing the chances that large errors are due to errors in the gold standard. The Metabolon platform allows for an unbiased examination of a large number of metabolites with identification of the leading metabolites.

The limitations of the study are mostly related to the steps one should take in making sure that a valid concept is rigorously tested in multiple clinical settings to allow an assessment of incremental clinical gain over current standards and cost effectiveness. First, the results should be validated in additional cohorts and robustness to special situations should be assessed, although we have used bootstrapping to make sure the current results a robust. It is also important to expect that prediction by eGFR will have a ceiling effect based on the quality of the gold standard which in most studies is likely to be less rigorous than in this discovery study which used an average of three consistent measured GFRs. Second, it will be important to determine the clinical factors, physiologic and pharmacologic, which influence any given analytes and robustness of any specific eGFR. However, we would propose that by using multiple analytes from different metabolic pathways, the overall eGFR would be less sensitive to the effect of any given non-GFR effect but this should be tested and quantified. We also propose that by having multiple analytes to choose from, it will be possible to minimize the risk of bias and error in a wider range of clinical settings. We also propose that the redundant information in multiple analytes in the eGFR can be used to exclude outlier analytes and produce an estimate, reflecting the average of the consistent analytes, which may be even more robust across a broad set of clinical settings. Third, some of the best metabolites (e.g., X-1564 and X-17299) are not yet named. However, their detailed mass spectrometry characteristics are known, documented in the Metabolon database, and they can be measured. Identification of these metabolite would allow for determination of absolute concentrations but the current paper shows that relative concentrations can yield useful results; pools of serum can be used to make sure calibration is consistent over time, even for unknown metabolites. Finally, assays for each analytes should be optimized and implemented in a setting which avoids drift over time. Initially, this can be done in a single laboratory, such as Metabolon's, but use across multiple laboratories should be associated with a standardization efforts comparable to what occurred for serum creatinine over the past decade.

The clinical applications of a precise eGFR are numerous and, in fact, it may be that many applications have been hampered by the current estimates having limited precision and limited robustness. First, clinical situations where muscle metabolism is altered make eGFRcr susceptible to error and indicate potential greater utility for an estimate based on other markers. Second, eGFR should be used whenever greater precision can improve patient care and minimize outcomes. The current error rates are not low (1-P30 of 10-40%), but we must recognize that in many cases nephrology care does not change across a relatively wide range of GFR. For example, blood pressure and glucose targets do not vary across relatively large GFR ranges. Toxic complications of drugs or contrast agents cleared by kidney filtration may very well benefit from improved GFR precision. Similarly, kidney transplant donors and recipients may benefit from eGFR with a low probability of having large errors. Some centers have implemented GFR measurements when greater accuracy is needed. These direct GFR measurements are based on injection of exogenous compounds (radioactive or not) but these often involve substantial burden in term of time (often requiring 4-6 hours) and can have limited precision due to incomplete bladder emptying in renal clearance estimates, non-renal clearance for blood clearance estimates and difficulties in standardization of the multiple steps and assays to obtain a measurement.

### CONCLUSIONS

Combination of multiple blood analytes based on a single blood draw can lead to a precise estimate of GFR (precise eGFR) of better precision than the current clinically used measures (eGFR using serum creatinine or even combined with serum cystatin C) and comparable (possibly better precision) than single measures of GFR (mGFR) using injection of exogenous substances. Different combinations of markers and algorithms allow for different desirable characteristics (e.g., metabolite only panel suitable for single platform analysis; obviating the need for clinical covariates; ability to exclude specific analytes; robustness to unreliability of one or more analytes). These methods can be tested in a range of clinical settings and using different measurement platforms to create new tests based on a single blood measure of comparable precision to GFR measurement using exogenous gold standards substantially improving the diagnosis, classification and prognostication for many individuals where current methods are inadequate.

TABLE 1
Characteristics of 188 AASK participants at the index visit*

| | Mean | |
|---|---|---|
| Characteristic | (SD) | Min-Max |
| Sex, male, % | 68 | |
| Age | 60 (9) | (29-74) |
| Serum creatinine, mg/dL | 2.0 (0.9) | (0.9-6.5) |
| Serum cystatin C, mg/dL | 1.8 (0.7) | (0.8-4.4) |
| mGFR, ml/min/1.73 m² | 48 (17) | (10-94) |
| mGFR at previous visit (42 month visit) | 47 (17) | (10-84) |
| mGFR at subsequent visit (54 month | 47 (17) | (9-96) |
| visit) | | |
| Average mGFR, ml/min/1.73 m2 | 47 (17) | (10-91) |
| (MGFR) | | |
| Systolic blood pressure, mmHg | 132 (12) | (109-163) |
| Diastolic blood pressure, mmHg | 80 (7) | (62-97) |
| Serum urea nitrogen, mg/dL | 25 (13) | (7-100) |

*Index visit is the AASK 48 month follow-up visit (F48). Participants with missing data on serum creatinine or cystatin at this visit were excluded (n = 12)

TABLE 2
Metabolites ranked by strength of negative correlation with average GFR

| | Correlation with MGFR | | | Correlation | | |
|---|---|---|---|---|---|---|
| | | | Adj. for | with | | |
| | | | Jaffe | demographics | | Biochemical name (X for |
| Metabolite # | r | p-value | creatinine | Age | Sex | unknown) |
| 545 | −0.808 | 0 | −0.44 | −0.05 | 0.04 | X-11564 |
| 186 | −0.787 | 0 | −0.45 | 0.02 | −0.01 | C-glycosyltryptophan* |
| 435 | −0.774 | 0 | −0.41 | −0.04 | 0.00 | pseudouridine |
| 746 | −0.768 | 0 | −0.33 | −0.03 | 0.13 | X-17299 |
| 374 | −0.766 | 0 | −0.50 | −0.04 | 0.06 | N-acetylthreonine |
| 373 | −0.758 | 0 | −0.39 | −0.01 | 0.15 | N-acetylserine |
| 241 | −0.758 | 0 | −0.37 | 0.07 | 0.04 | erythritol |
| 161 | −0.739 | 0 | −0.35 | −0.02 | 0.03 | arabitol |
| 499 | −0.733 | 0 | −0.38 | −0.03 | −0.03 | urea |
| 714 | −0.732 | 0 | −0.28 | −0.05 | 0.13 | X-16394 |
| 525 | −0.730 | 0 | −0.26 | 0.04 | 0.04 | X-11423 |
| 242 | −0.718 | 0 | −0.28 | 0.04 | 0.01 | erythronate* |
| 214 | −0.710 | 0 | −0.11 | −0.09 | 0.24 | creatinine |
| 359 | −0.703 | 0 | −0.25 | 0.03 | 0.01 | myo-inositol |
| 385 | −0.699 | 0 | −0.25 | −0.01 | 0.09 | N6-carbamoylthreonyladenosine |
| 618 | −0.683 | 0 | −0.17 | 0.00 | 0.00 | X-12749 |
| 576 | −0.683 | 0 | −0.42 | −0.02 | −0.04 | X-12104 |
| 366 | −0.682 | 0 | −0.41 | −0.03 | 0.12 | N-acetylalanine |
| 382 | −0.678 | 0 | −0.32 | −0.05 | 0.04 | N2,N2-dimethylguanosine |
| 114 | −0.667 | 0 | −0.14 | −0.01 | 0.03 | 4-acetamidobutanoate |
| 566 | −0.658 | 0 | −0.24 | −0.04 | 0.08 | X-11945 |
| 26 | −0.644 | 0 | −0.30 | 0.01 | 0.16 | 1-methylhistidine |
| 162 | −0.637 | 0 | −0.13 | −0.01 | 0.02 | arabonate |
| 375 | −0.635 | 0 | −0.39 | 0.00 | 0.00 | N-formylmethionine |
| 69 | −0.633 | 0 | −0.33 | −0.09 | 0.12 | 2-hydroxyisobutyrate |
| 510 | −0.614 | 0 | −0.12 | −0.04 | −0.02 | xylonate |
| 469 | −0.609 | 0 | −0.32 | −0.08 | 0.00 | succinylcarnitine |
| 371 | −0.604 | 0 | −0.19 | −0.05 | 0.06 | N-acetylneuraminate |
| 603 | −0.600 | 0 | −0.18 | −0.05 | 0.03 | X-12686 |
| 363 | −0.597 | 0 | −0.06 | −0.04 | 0.06 | N-acetyl-1-methylhistidine* |
| 298 | −0.593 | 0 | −0.24 | 0.04 | −0.06 | homocitrulline |
| 775 | −0.590 | 0 | −0.25 | 0.10 | −0.01 | X-17703 |
| 531 | −0.575 | 0 | −0.21 | 0.09 | 0.07 | X-11444 |
| 480 | −0.568 | 0 | −0.05 | −0.03 | −0.01 | threitol |
| 797 | −0.566 | 0 | −0.39 | 0.02 | −0.16 | X-18887 |
| 632 | −0.565 | 0 | −0.26 | 0.17 | 0.07 | X-12846 |
| 399 | −0.563 | 0 | −0.27 | 0.21 | −0.12 | p-cresol sulfate |
| 110 | −0.557 | 0 | −0.18 | 0.07 | −0.12 | 3-methylglutarylcarnitine (C6) |
| 379 | −0.557 | 0 | −0.27 | −0.03 | −0.11 | N1-Methyl-2-pyridone-5-carboxamide |
| 271 | −0.552 | 0 | −0.18 | −0.07 | 0.10 | glutarylcarnitine (C5) |
| 729 | −0.550 | 0 | −0.21 | −0.01 | 0.14 | X-16982 |
| 319 | −0.550 | 0 | −0.28 | 0.07 | −0.05 | isobutyrylcarnitine |
| 104 | −0.549 | 0 | −0.15 | 0.07 | −0.09 | 3-indoxyl sulfate |
| 755 | −0.545 | 0 | −0.11 | 0.12 | −0.02 | X-17357 |
| 251 | −0.543 | 2.22E−16 | −0.20 | 0.01 | 0.02 | galaclitol (dulcitol) |
| 625 | −0.543 | 2.22E−16 | −0.06 | −0.01 | 0.01 | X-12822 |
| 651 | −0.539 | 2.22E−16 | −0.13 | −0.09 | 0.02 | X-13837 |
| 514 | −0.529 | 1.11E−15 | −0.26 | −0.11 | −0.08 | X-02249 |
| 596 | −0.528 | 1.33E−15 | −0.12 | 0.04 | −0.02 | X-12411 |
| 652 | −0.528 | 1.33E−15 | −0.12 | −0.05 | 0.03 | X-13844 |
| 326 | −0.527 | 1.55E−15 | −0.35 | −0.02 | −0.05 | kynurenine |
| 567 | −0.523 | 2.89E−15 | −0.01 | −0.08 | 0.01 | X-12007 |
| 643 | −0.520 | 4.66E−15 | −0.11 | −0.06 | 0.13 | X-13553 |
| 580 | −0.517 | 6.88E−15 | 0.00 | 0.01 | 0.02 | X-12125 |
| 383 | −0.516 | 7.77E−15 | −0.09 | −0.06 | 0.11 | N2,N5-diacetylornithine |
| 390 | −0.516 | 7.99E−15 | −0.12 | 0.04 | −0.12 | O-methylcatechol sulfate |
| 650 | −0.509 | 2.35E−14 | 0.02 | −0.17 | 0.12 | X-13835 |
| 609 | −0.504 | 4.62E−14 | 0.04 | −0.19 | 0.14 | X-12729 |
| 621 | −0.500 | 7.88E−14 | 0.02 | −0.04 | 0.03 | X-12814 |

TABLE 3
Metabolites ranked by strength of positive correlation with average GFR

| | Correlation with average | | | | | |
|---|---|---|---|---|---|---|
| | mGFR | | | | | |
| Metabolite | | | Adj. Jaffe | Correlation | | Biochemical name (X for |
| # | r | p-value | creatinine | Age | Sex | unknown) |
| 501 | 0.400 | 8.13E−09 | 0.29 | −0.03 | 0.11 | valine |
| 495 | 0.409 | 3.35E−09 | 0.24 | 0.00 | 0.08 | tyrosine |
| 124 | 0.426 | 6.00E−10 | 0.31 | 0.00 | 0.24 | 4-methyl-2-oxopentanoate |
| 276 | 0.460 | 1.37E−11 | 0.27 | 0.03 | 0.07 | glycerophosphorylcholine (GPC) |
| 500 | 0.466 | 6.30E−12 | 0.25 | −0.05 | 0.11 | undine |
| 482 | 0.474 | 2.33E−12 | 0.29 | −0.01 | 0.11 | threonine |
| 816 | 0.476 | 1.89E−12 | 0.19 | 0.01 | 0.14 | X-19380 |
| 817 | 0.528 | 1.33E−15 | 0.32 | −0.04 | 0.12 | X-19411 |
| 492 | 0.552 | 0 | 0.33 | −0.03 | 0.20 | tryptophan |

TABLE 4
Prediction of GFR using different estimates

| | Without age and sex | | | | With age and sex | | | |
|---|---|---|---|---|---|---|---|---|
| | RMSE | 1-P30 | 1-P20 | 1-P10 | RMSE | 1-P30 | 1-P20 | 1-P10 |
| eGFR cr¹ | | | | | 0.201 | 8.0% | 25.0% | 59.0% |
| eGFR cys¹ | | | | | 0.208 | 6.9% | 28.7% | 63.8% |
| eGFR cr + cys¹ | | | | | 0.170 | 4.8% | 20.2% | 56.4% |
| bio_214 (creatinine) | 0.286 | 29.8% | 45.2% | 70.7% | 0.263 | 23.9% | 41.0% | 68.1% |
| Creatinine (Jaffe) | 0.227 | 17.0% | 36.2% | 64.9% | 0.192 | 8.5% | 27.7% | 54.8% |
| Cystatin C | 0.168 | 9.0% | 20.7% | 53.7% | 0.165 | 8.5% | 18.6% | 47.9% |
| Creatinine (Jaffe) + | 0.155 | 5.9% | 20.7% | 47.9% | 0.140 | 4.3% | 12.2% | 46.8% |
| Cystatin C | | | | | | | | |
| bio_545 (X-11564) | 0.173 | 6.9% | 25.0% | 60.1% | 0.164 | 5.9% | 19.1% | 60.1% |
| bio_186 (C-glycosyl- | 0.179 | 7.4% | 25.0% | 61.7% | 0.179 | 6.9% | 23.4% | 61.2% |
| tryptophan*) | | | | | | | | |
| bio_435 (pseudo- | 0.227 | 14.4% | 38.3% | 64.4% | 0.226 | 12.8% | 34.0% | 63.3% |
| undine) | | | | | | | | |
| bio_746 (X-17299) | 0.253 | 26.6% | 41.5% | 66.5% | 0.243 | 26.1% | 42.6% | 62.8% |
| bio_374 (N-acetyl-threonine) | 0.253 | 21.3% | 39.9% | 64.9% | 0.251 | 21.3% | 38.8% | 62.2% |
| Top 5 Metabolites¹ | 0.145*** | 3.2%* | 14.9%** | 48.9%* | 0.138*** | 2.1%** | 12.8%*** | 46.8%* |
| Top 10 Metabolites¹ | 0.142*** | 2.7%* | 14.4%** | 46.8%* | 0.136*** | 2.1%** | 9.6%*** | 45.2%** |
| Creatinine + Cystatin C + | 0.139*** | 1.6%** | 12.2%*** | 47.3%* | 0.128*** | 1.1%*** | 10.6%*** | 41.0%*** |
| top 5 Metabolites | | | | | | | | |
| Best by Stepwise (6) (7) | 0.139*** | 2.7%* | 12.2%*** | 45.2%** | 0.130*** | 1.1%*** | 8.0%*** | 46.3%* |
| Best by Stepwise, | 0.124*** | 1.1%*** | 9.0%*** | 41.0%*** | 0.114*** | 0.5%*** | 5.9%*** | 37.2%*** |
| p_enter(0.05) | | | | | | | | |
| p_exit(0.1)(14) (15) | | | | | | | | |
| Best by Stepwise | 0.138*** | 0.5%*** | 14.4%** | 50.5% | 0.125*** | 1.1%*** | 9.6%*** | 42.0%*** |
| considering Cr (5) (6) | | | | | | | | |
| Creatinine + best by | 0.137*** | 2.1%** | 12.2%*** | 44.7%** | 0.127*** | 1.1%*** | 9.0%*** | 43.1%*** |
| stepwise (6) (7) | | | | | | | | |
| Best by Stepwise | 0.134*** | 2.7%* | 10.1%*** | 46.3%* | 0.127*** | 1.1%*** | 11.7%*** | 41.5%*** |
| considering Cr + Cys (5) | | | | | | | | |
| (3) | | | | | | | | |

*p ≦ 0.05,
**p ≦ 0.01,
***p ≦ 0.001 compared to eGFRcr. Significance testing only for lower panel of the table.
¹Previously developed eGFR estimates already include age and sex (race is set to African-American for all participants) as well as a spline (nearly all participants are above the knots for creatinine and cystatin C). Prediction statistics are calculated based on the eGFR itself (equivalent to having an intercept of zero and slope of 1).
² Top metabolites are based on the correlation rank order listed in Table 2 (first 5 or 10).
Stepwise regression models list the number of variables selected in parentheses with the model without demographics listed first. Default p-value for entering is 0.05 and 0.01 for exist so all variables are p < 0.01; more liberal criteria model performance (p-exit = 0.10) are also shown. Variables selected as best by stepwise considering creatinine have excellent performance and feasibility on a single assay (# indicates rank of the correlation in Table 13): X-11564 (#1), C-glycosyltryptophan (#2), Leucine (#750 positive correlation with mGFR), 1-methylhistidine (#22), 1-myristoylglycerophosphocholine (14:0) ((#735 positive correlation with mGFR); when adding age & sex the model adds: X-18914 (#733).

TABLE 5
Prediction of GFR using different estimates-limited to known metabolites

| | Without age and sex | | | | With age and sex | | | |
|---|---|---|---|---|---|---|---|---|
| | RMSE | 1-P30 | 1-P20 | 1-P10 | RMSE | 1-P30 | 1-P20 | 1-P10 |
| eGFR cr | | | | | 0.201 | 8.0% | 25.0% | 59.0% |
| eGFR cys | | | | | 0.208 | 6.9% | 28.7% | 63.8% |
| eGFR cr + cys | | | | | 0.170 | 4.8% | 20.2% | 56.4% |
| bio_214 (creatinine) | 0.286 | 29.8% | 45.2% | 70.7% | 0.263 | 23.9% | 41.0% | 68.1% |
| Creatinine (Jaffe) | 0.227 | 17.0% | 36.2% | 64.9% | 0.192 | 8.5% | 27.7% | 54.8% |
| Cystatin C | 0.168 | 9.0% | 20.7% | 53.7% | 0.165 | 8.5% | 18.6% | 47.9% |
| Creatinine + Cystatin C | 0.155 | 5.9% | 20.7% | 47.9% | 0.140 | 4.3% | 12.2% | 46.8% |
| bio_186 (C-glycosyl- | 0.179 | 7.4% | 25.0% | 61.7% | 0.179 | 6.9% | 23.4% | 61.2% |
| tryptophan*) | | | | | | | | |
| bio_435 (pseudouridine) | 0.227 | 14.4% | 38.3% | 64.4% | 0.226 | 12.8% | 34.0% | 63.3% |
| bio_374 (N-acetyl-threonine) | 0.253 | 21.3% | 39.9% | 64.9% | 0.251 | 21.3% | 38.8% | 62.2% |
| bio_373 (N-acetylserine) | 0.247 | 18.6% | 35.6% | 62.8% | 0.241 | 18.1% | 33.0% | 64.4% |
| bio_241 (erythritol) | 0.217 | 17.0% | 36.2% | 61.2% | 0.216 | 16.5% | 36.7% | 62.8% |
| Top 5 Metabolites | 0.158*** | 3.2%* | 21.8% | 52.1% | 0.156*** | 4.3% | 20.2% | 51.1% |
| Top 10 Metabolites | 0.148*** | 1.6%** | 18.1% | 47.3%* | 0.142*** | 1.1%*** | 13.8%** | 46.3%** |
| Creatinine + Cystatin C + | 0.140*** | 2.1%** | 13.3%*** | 48.9%* | 0.128*** | 2.7%** | 11.2%*** | 39.9%*** |
| top 5 Metabolites | | | | | | | | |
| Best by Stepwise (5) (7) | 0.148*** | 4.3% | 15.4%* | 52.1% | 0.140*** | 1.1%*** | 15.4%* | 46.3%** |
| Best by Stepwise, | 0.129*** | 1.1%*** | 10.6%*** | 42.6%** | 0.126*** | 1.6%*** | 8.0%*** | 36.7%*** |
| p_enter(0.05) | | | | | | | | |
| p_exit(0.1)(14) (14) | | | | | | | | |
| Best by Stepwise | 0.144*** | 1.6%** | 16.5%* | 49.5%* | 0.136*** | 1.1%*** | 13.8%*** | 45.2%** |
| considering Cr (4) (3) | | | | | | | | |
| Creatinine + best by | 0.143*** | 2.1%** | 14.9%** | 52.1% | 0.135*** | 1.1%*** | 11.7%*** | 44.1%** |
| stepwise above (5) (7) | | | | | | | | |
| Best by Stepwise | 0.134*** | 2.1%** | 12.2%*** | 47.3%* | 0.129*** | 2.1%** | 12.2%*** | 41.0%*** |
| considering Cr + Cys (4) | | | | | | | | |
| (2) | | | | | | | | |
| Creatinine + Cystatin C + | 0.135*** | 2.7%* | 12.8%*** | 43.6%** | 0.130*** | 2.1%** | 10.1%*** | 42.6%*** |
| best by stepwise above (5) | | | | | | | | |
| (7) | | | | | | | | |

*p ≦ 0.05,
**p ≦ 0.01,
***p ≦ 0.001 compared to eGFRcr. Significance testing only for lower panel of the table.
¹ Previously developed eGFR estimates already include age and sex (race is set to African-American for all participants) as well as a spline (nearly all participants are above the knots for creatinine and cystatin C). Prediction statistics are calculated based on the eGFR itself (equivalent to having an intercept of zero and slope of 1).
² Top metabolites are based on the correlation rank order of KNOWN metabolites listed in Table 2 (first 5 or 10).

Stepwise regression models list the number of variables selected in parentheses with the model without demographics listed first. Default p-value for entering is 0.05 and 0.01 for exist so all variables are p<0.01; more liberal criteria model performance (p-exit=0.10) are also shown. Variables selected as best by stepwise considering creatinine have excellent performance and feasibility on a single assay (# indicates rank of the correlation in Table 13): C-glycosyltryptophan (#2), pseudouridine (#3), myo-inositol (#14), phenylacetylglutamine (#65); when adding age & sex the model adds: N-acetylserine (#6) but drops myo-inositol (#14), phenylacetylglutamine (#65).

TABLE 6
Diagnostic performance of CKD (average mGFR <60 ml/min/1.73 m²)
measured by area under the curve (AUC), sensitivity (Sn) and specificity (Sp)
among participants with average mGFR of 45-90 ml/min/1.73 m².

| | Without age and sex | | | With age and sex | | |
|---|---|---|---|---|---|---|
| cut off 60, range 45-90 | AUC | Sn | Sp | AUC | Sn | Sp |
| eGFR cr | | | | 0.792 | 83.8% | 48.8% |
| eGFR cys | | | | 0.846 | 95.6% | 46.3% |
| eGFR cr + cys | | | | 0.869 | 92.6% | 48.8% |
| bio_214 (creatinine) | 0.712 | 85.3% | 31.7% | 0.764 | 91.2% | 43.9% |
| Creatinine (Jaffe) | 0.700 | 70.6% | 46.3% | 0.794 | 83.8% | 46.3% |
| Cystatin C | 0.827 | 82.4% | 61.0% | 0.843 | 85.3% | 65.9% |
| Creatinine + Cystatin C | 0.829 | 80.9% | 65.9% | 0.871 | 86.8% | 73.2% |
| bio_545 (X-11564) | 0.759 | 77.9% | 51.2% | 0.793 | 77.9% | 53.7% |
| bio_186 (C-glycosyltryptophan*) | 0.794 | 80.9% | 46.3% | 0.798 | 80.9% | 41.5% |
| bio_435 (pseudouridine) | 0.744 | 85.3% | 39.0% | 0.745 | 85.3% | 43.9% |
| bio_746 (X-17299) | 0.664 | 76.5% | 43.9% | 0.684 | 79.4% | 46.3% |
| bio_374 (N-acetylthreonine) | 0.783 | 83.8% | 46.3% | 0.791 | 83.8% | 46.3% |
| Top 5 Metabolites | 0.825 | 83.8% | 65.9% | 0.858 | 80.9% | 63.4% |
| Top 10 Metabolites | 0.848 | 80.9% | 68.3% | 0.869 | 83.8% | 75.6% |
| Best by Stepwise (6) (7) | 0.843 | 79.4% | 68.3% | 0.871 | 82.4% | 75.6% |
| Best by Stepwise, p_enter(0.05) | 0.882 | 85.3% | 78.0% | 0.900 | 89.7% | 80.5% |
| p_exit(0.1)(14) (15) | | | | | | |
| Best by Stepwise considering Cr (5) (6) | 0.841 | 76.5% | 68.3% | 0.872 | 79.4% | 68.3% |
| Creatinine + best by stepwise above (6) | 0.844 | 79.4% | 68.3% | 0.878 | 85.3% | 75.6% |
| (7) | | | | | | |
| Best by Stepwise considering Cr + Cys (5) | 0.860 | 82.4% | 63.4% | 0.886 | 86.8% | 70.7% |
| (3) | | | | | | |
| Creatinine + Cystatin C + top 5 | 0.851 | 83.8% | 65.9% | 0.880 | 80.9% | 73.2% |
| Metabolites | | | | | | |
| Creatinine + Cystatin C + best by | 0.865 | 76.5% | 63.4% | 0.890 | 85.3% | 75.6% |
| stepwise above (6) (7) | | | | | | |

Models correspond to those in Table 4

TABLE 7
Diagnostic performance of distinguishing CKD stage G3B (average mGFR 30
to <45 ml/min/1.73 m²) from G3A (average mGFR 45 to <60 ml/min/1.73
m²) measured by area under the curve (AUC), sensitivity (Sn) and specificity
(Sp) among participants with average mGFR of 30-60 ml/min/1.73 m².

| | Without age and sex | | | With age and sex | | |
|---|---|---|---|---|---|---|
| cut off 45, range 30-60 | AUC | Sn | Sp | AUC | Sn | Sp |
| eGFR cf | | | | 0.925 | 95.1% | 76.5% |
| eGFR cys | | | | 0.912 | 92.7% | 57.4% |
| eGFR cr + cys | | | | 0.960 | 95.1% | 67.6% |
| bio_214 (creatinine) | 0.806 | 82.9% | 61.8% | 0.820 | 82.9% | 70.6% |
| Creatinine (Jaffe) | 0.879 | 80.5% | 67.6% | 0.926 | 87.8% | 76.5% |
| Cystatin C | 0.912 | 87.8% | 77.9% | 0.916 | 87.8% | 80.9% |
| Creatinine + Cystatin C | 0.936 | 87.8% | 79.4% | 0.958 | 87.8% | 82.4% |
| bio_545 (X-11564) | 0.878 | 80.5% | 79.4% | 0.885 | 78.0% | 79.4% |
| bio_186 (C-glycosyltryptophan*) | 0.856 | 75.6% | 76.5% | 0.854 | 78.0% | 73.5% |
| bio_435 (pseudouridine) | 0.814 | 75.6% | 76.5% | 0.816 | 80.5% | 75.0% |
| bio_746 (X-17299) | 0.897 | 87.8% | 70.6% | 0.901 | 85.4% | 73.5% |
| bio_374 (N-acetylthreonine) | 0.780 | 80.5% | 70.6% | 0.761 | 78.0% | 64.7% |
| Top 5 Metabolites | 0.942 | 87.8% | 88.2% | 0.950 | 87.8% | 88.2% |
| Top 10 Metabolites | 0.936 | 87.8% | 88.2% | 0.946 | 87.8% | 85.3% |
| Best by Stepwise (6) (7) | 0.933 | 85.4% | 85.3% | 0.951 | 90.2% | 89.7% |
| Best by Stepwise, p_enter(0.05) | 0.961 | 92.7% | 85.3% | 0.968 | 95.1% | 91.2% |
| p_exit(0.1)(14) (15) | | | | | | |
| Best by Stepwise considering Cr (5) (6) | 0.915 | 87.8% | 76.5% | 0.941 | 87.8% | 82.4% |
| Creatinine + best by stepwise above (6) | 0.941 | 87.8% | 83.8% | 0.957 | 90.2% | 89.7% |
| (7) | | | | | | |
| Best by Stepwise considering Cr + Cys (5) | 0.932 | 85.4% | 83.8% | 0.951 | 87.8% | 83.8% |
| (3) | | | | | | |
| Creatinine + Cystatin C + top 5 | 0.951 | 95.1% | 85.3% | 0.962 | 90.2% | 86.8% |
| Metabolites | | | | | | |
| Creatinine + Cystatin C + best by | 0.950 | 90.2% | 85.3% | 0.963 | 90.2% | 88.2% |
| stepwise above (6) (7) | | | | | | |

### Models Correspond to Those in Table 4

TABLE 8
Diagnostic performance of CKD (average mGFR <60 ml/min/1.73 m²)
measured by area under the curve (AUC), sensitivity (Sn) and specificity (Sp)
among participants with average mGFR of 45-90 ml/min/1.73 m².

| | Without age and sex | | | With age and sex | | |
|---|---|---|---|---|---|---|
| cut off 60, range 45-90 | AUC | Sn | Sp | AUC | Sn | Sp |
| eGFR cr | | | | 0.792 | 83.8% | 48.8% |
| eGFR cys | | | | 0.846 | 95.6% | 46.3% |
| eGFR cr + cys | | | | 0.869 | 92.6% | 48.8% |
| bio_214 (creatinine) | 0.712 | 85.3% | 31.7% | 0.764 | 91.2% | 43.9% |
| Creatinine (Jaffe) | 0.700 | 70.6% | 46.3% | 0.794 | 83.8% | 46.3% |
| Cystatin C | 0.827 | 82.4% | 61.0% | 0.843 | 85.3% | 65.9% |
| Creatinine + Cystatin C | 0.829 | 80.9% | 65.9% | 0.871 | 86.8% | 73.2% |
| bio_186 (C-glycosyltryptophan*) | 0.759 | 77.9% | 51.2% | 0.793 | 77.9% | 53.7% |
| bio_435 (pseudouridine) | 0.794 | 80.9% | 46.3% | 0.798 | 80.9% | 41.5% |
| bio_374 (N-acetylthreonine) | 0.744 | 85.3% | 39.0% | 0.745 | 85.3% | 43.9% |
| bio_373 (N-acetylserine) | 0.773 | 85.3% | 53.7% | 0.775 | 86.8% | 56.1% |
| bio_241 (erythritol) | 0.818 | 85.3% | 58.5% | 0.826 | 86.8% | 58.5% |
| Top 5 Metabolites | 0.848 | 82.4% | 65.9% | 0.860 | 83.8% | 63.4% |
| Top 10 Metabolites | 0.869 | 85.3% | 78.0% | 0.906 | 88.2% | 73.2% |
| Best by Stepwise (6) (7) | 0.844 | 82.4% | 63.4% | 0.865 | 83.8% | 65.9% |
| Best by Stepwise, p_enter(0.05) | 0.901 | 82.4% | 68.3% | 0.901 | 82.4% | 78.0% |
| p_exit(0.1)(14) (15) | | | | | | |
| Best by Step-wise considering Cr (4) (3) | 0.850 | 79.4% | 68.3% | 0.869 | 86.8% | 73.2% |
| Creatinine + best by stepwise above (6) | 0.851 | 79.4% | 68.3% | 0.861 | 82.4% | 68.3% |
| (7) | | | | | | |
| Best by Stepwise considering Cr + Cys (4) | 0.880 | 79.4% | 75.6% | 0.886 | 88.2% | 73.2% |
| (2) | | | | | | |
| Creatinine + Cystatin C + top 5 | 0.865 | 82.4% | 65.9% | 0.894 | 88.2% | 70.7% |
| Metabolites | | | | | | |
| Creatinine + Cystatin C + best by | 0.872 | 83.8% | 73.2% | 0.892 | 82.4% | 73.2% |
| stepwise above (6) (7) | | | | | | |

Models correspond to those in Table 5

TABLE 9
Diagnostic performance of distinguishing CKD stage G3B (average mGFR 30
to <45 ml/min/1.73 m²) from G3A (average mGFR 45 to <60 ml/min/1.73
m²) measured by area under the curve (AUC), sensitivity (Sn) and specificity
(Sp) among participants with average mGFR of 30-60 ml/min/1.73 m².

| | Without age and sex | | | With age and sex | | |
|---|---|---|---|---|---|---|
| cut off 45, range 30-60 | AUC | Sn | Sp | AUC | Sn | Sp |
| eGFR cr | | | | 0.925 | 95.1% | 76.5% |
| eGFR cys | | | | 0.912 | 92.7% | 57.4% |
| eGFR cr + cys | | | | 0.960 | 95.1% | 67.6% |
| bio_214 (creatinine) | 0.806 | 82.9% | 61.8% | 0.820 | 82.9% | 70.6% |
| Creatinine (Jaffe) | 0.879 | 80.5% | 67.6% | 0.926 | 87.8% | 76.5% |
| Cystatin C | 0.912 | 87.8% | 77.9% | 0.916 | 87.8% | 80.9% |
| Creatinine + Cystatin C | 0.936 | 87.8% | 79.4% | 0.958 | 87.8% | 82.4% |
| bio_186 (C-glycosyltryptophan*) | 0.878 | 80.5% | 79.4% | 0.885 | 78.0% | 79.4% |
| bio_435 (pseudouridine) | 0.856 | 75.6% | 76.5% | 0.854 | 78.0% | 73.5% |
| bio_374 (N-acetylthreonine) | 0.814 | 75.6% | 76.5% | 0.816 | 80.5% | 75.0% |
| bio_373 (N-acetylserine) | 0.751 | 78.0% | 64.7% | 0.756 | 80.5% | 69.1% |
| bio_241 (erythritol) | 0.811 | 80.5% | 60.3% | 0.813 | 75.6% | 64.7% |
| Top 5 Metabolites | 0.883 | 80.5% | 80.9% | 0.882 | 78.0% | 77.9% |
| Top 10 Metabolites | 0.906 | 75.6% | 83.8% | 0.911 | 78.0% | 86.8% |
| Best by Stepwise (6) (7) | 0.916 | 78.0% | 83.8% | 0.925 | 85.4% | 86.8% |
| Best by Stepwise, p_enter(0.05) | 0.918 | 75.6% | 86.8% | 0.934 | 87.8% | 83.8% |
| p_exit(0.1)(14) (15) | | | | | | |
| Best by Stepwise considering Cr (4) (3) | 0.940 | 90.2% | 86.8% | 0.946 | 85.4% | 89.7% |
| Creatinine + best by stepwise above (6) | 0.943 | 87.8% | 89.7% | 0.949 | 87.8% | 88.2% |
| (7) | | | | | | |
| Best by Stepwise considering Cr + Cys (4) | 0.939 | 87.8% | 82.4% | 0.950 | 90.2% | 85.3% |
| (2) | | | | | | |
| Creatinine + Cystatin C + top 5 | 0.938 | 90.2% | 86.8% | 0.958 | 90.2% | 88.2% |
| Metabolites | | | | | | |
| Creatinine + Cystatin C + best by | 0.949 | 92.7% | 86.8% | 0.950 | 87.8% | 86.8% |
| stepwise above (6) (7) | | | | | | |

Models correspond to those in Table 5.

TABLE 10
Characteristics of unnamed metabolites*

| BIOCHEMICAL | LIB_ID | COMP_ID | QUANT | RT | SPECTRA |
|---|---|---|---|---|---|
| Unknown - 11945 | 200 | 33290 | 283.1 | 1.83 | 126.2:0.1 151.1::100 152:0.1 195.2:0.1 206.1:0.2 |
| | | | | | 222.1:0.1 223.1:0.2 264:0.1 265.1:0.3 266.1:0.1 |
| Unknown - 12104 | 200 | 33519 | 271.1 | 1.72 | 114.1:0.2 122.1:5.4 133.1:0.3 139.1:100 140.1:0.9 |
| | | | | | 211.1:0.1 214.1:0.1 227.1:0.2 252.2:0.2 253.1:0.4 |
| | | | | | 254.1:0.2 |
| Unknown - 12686 | 200 | 34295 | 181.1 | 1.09 | 61.1:1.5 65.1:0.7 69.1:5.8 71.1:0.5 75.1:0.8 81.1:0.7 |
| | | | | | 85.1:1 87.1:1 97.1:2.2 99.1:3.9 101.1:0.2 103.1:3.8 |
| | | | | | 105.1:0.7 107.1:0.3 115.1:6.6 117.1:3.9 121.2:0.1 |
| | | | | | 127.1:0.2 133.1:21.7 134.1:7.5 135.2:1.6 136.2:0.5 |
| | | | | | 138.1:0.2 145.1:5.5 149.1:0.5 152.1:0.2 153.2:0.2 |
| | | | | | 154.2:0.2 161.1:0.3 163:100 164.1:0.7 |
| Unknown - 12749 - | 200 | 34359 | 262.1 | 1.51 | 85.1:2.1 130.2:0.5 136.2:0.9 144.2:1.1 165.1:4.7 |
| retired - combo of | | | | | 166.2:0.3 182.1:11.1 183.2:0.5 203.1:0.9 216.1:100 |
| metabolites | | | | | 217.1:5.1 218.2:3.5 219.2:0.4 225.2:0.3 226.2:0.4 |
| | | | | | 226.9:0.3 243.1:0.5 245:5.1 246.1:0.3 |
| Unknown - 16394 | 200 | 38963 | 229.2 | 1.59 | 70:20.2 71:1.2 83:0.3 98:0.4 112.1:1.8 114.1:3.9 124:8 |
| | | | | | 125.1:0.6 126.1:2.7 132:1.3 142:100 143.1:8.4 145.1:0.2 |
| | | | | | 155:1.1 158.1:0.5 159.1:0.2 169.1:2.1 170:8.5 171:1.1 |
| | | | | | 173:0.6 183.1:0.7 186.1:0.6 187.1:0.3 196:0.4 200.1:0.2 |
| | | | | | 201.2:0.7 210.1:1.7 211.1:3.5 212.1:1 229.2:2.8 |
| | | | | | 230.2:0.3 |
| Unknown - 16982 | 200 | 39568 | 191.9 | 1.53 | 60:0.5 61:0.6 73:0.5 99:0.3 101:4.7 102.1:0.4 105:0.2 |
| | | | | | 107.1:0.4 108.1:0.4 109.1:0.3 114.1:0.4 115.1:1 |
| | | | | | 116.1:0.3 117.1:0.4 118.1:0.4 119.1:1.5 120.1:0.3 |
| | | | | | 121.1:0.9 122:0.4 124.1:0.2 127.1:0.4 128.1:0.5 |
| | | | | | 129.1:0.5 130.1:0.5 132.1:100 133.1:1.1 135.1:1.9 |
| | | | | | 136.1:0.5 140.1:1 141.1:1.2 142.1:0.6 145:9.6 146:4.5 |
| | | | | | 147:1.9 148.1:1.7 149.1:1.9 150.1:0.7 155.2:0.7 |
| | | | | | 156.1:0.7 157.1:0.5 159:4.5 160:13.9 161.1:0.5 163.1:2.6 |
| | | | | | 164.1:2.8 173:10.5 174.1:13.4 175.1:13.4 178:0.8 |
| | | | | | 213.2:0.3 |
| Unknown - 17299 | 200 | 40097 | 229.2 | 1.2 | 68:0.2 70:19.5 71:0.6 96:9.6 114:4.3 116.1:0.2 124:7.6 |
| | | | | | 125.1:0.3 126.1:2.8 132.1:0.7 142:100 143.1:5.1 152:0.2 |
| | | | | | 158.1:0.3 169:8 170:8.8 171:0.5 201.1:0.3 229.2:0.5 |
| Unknown - 02249 | 201 | 32587 | 267.2 | 4.03 | 179.3:1.1 180.3:0.1 205.1:0.4 223.1:100 224.2:6.2 |
| | | | | | 239.2:0.4 249.1:2.4 250.1:0.2 |
| Unknown - 11423 - | 201 | 32740 | 260.1 | 1.05 | 79.1:0.2 80.1:0.3 81.1:0.3 93.2:0.2 96.1:0.2 97.1:0.2 |
| retired for O-sulfo- | | | | | 119.2:2.3 120.2:0.3 134.2:0.4 135.1:0.8 136.3:0.3 |
| L-tyrosine | | | | | 137.1:1 142.1:0.2 153:0.2 155.3:0.1 161:0.3 163.1:0.5 |
| | | | | | 169.2:0.3 170.3:0.2 171.2:0.3 173.2:0.3 174:0.3 |
| | | | | | 175.1:0.2 176.1:0.1 178.9:0.3 180.1:12 181.1:6.7 |
| | | | | | 186.1:3.1 187.1:0.8 189.2:0.5 190.1:0.3 191.1:0.2 |
| | | | | | 192.1:0.2 193.2:0.2 196.2:0.3 197.2:0.4 199:100 |
| | | | | | 200.1:9.5 201.2:0.5 203.9:0.2 205:0.7 213.2:0.5 |
| | | | | | 213.9:0.3 215:29.2 216.1:3.4 217.1:0.3 219.3:0.2 221:0.2 |
| | | | | | 223.1:0.2 227.3:0.2 231.1:0.2 232.4:0.3 233.2:0.5 |
| | | | | | 241.1:0.4 242.3:1.5 242.9:7.9 244:0.8 245.1:0.2 |
| | | | | | 259.1:0.3 260.1:0.4 261.2:0.3 |
| Unknown - 11444 | 201 | 32761 | 541.2 | 3.99 | 157.1:1 175.1:1.1 176:0.7 241:1.2 271.3:0.4 279.2:0.6 |
| | | | | | 281.3:0.3 283.2:0.8 287.3:0.4 289.2:0.9 291.3:0.7 |
| | | | | | 298.2:2 299.2:1.1 300.2:0.7 301.3:3.7 302.3:1.1 |
| | | | | | 305.3:0.6 306.3:0.5 307.2:1.4 308.3:0.7 315.3:0.5 |
| | | | | | 317.3:3.4 318.3:1 319.3:0.7 320.3:0.5 329.2:0.7 |
| | | | | | 330.4:0.7 332.5:0.5 333.3:0.6 335.3:12.3 336.3:2.2 |
| | | | | | 345.3:0.6 347.3:1.8 348.3:0.6 357:0.6 358.2:0.6 |
| | | | | | 359.4:0.8 360.3:0.5 361.2:0.7 363.3:1.3 364.2:0.7 |
| | | | | | 365.3:0.7 366.4:0.4 371.3:0.7 372.3:0.5 373.3:0.5 |
| | | | | | 374.2:0.5 375.1:0.6 376.2:0.4 377.2:2 378.3:0.5 379:0.4 |
| | | | | | 386.7:0.7 387.4:0.4 389.3:1.6 390.3:0.8 391.3:1 |
| | | | | | 392.1:0.5 393.3:1.6 394.2:0.7 400.9:2 401.5:0.8 |
| | | | | | 402.1:0.3 403.2:0.5 404.2:0.7 405.3:2.8 406.3:0.7 |
| | | | | | 413.3:1 415.2:2.2 416.3:0.9 417.3:0.7 418.3:0.6 |
| | | | | | 419.2:0.6 423.3:6.4 424.3:1.6 427.2:0.5 428.4:0.4 |
| | | | | | 431.3:0.3 432.4:1 433.3:0.3 434.1:0.9 435.2:1 436.9:7 |
| | | | | | 443.4:0.7 446.1:1 447.3:0.6 448:1.1 449.3:12.3 450.3:2.9 |
| | | | | | 451.2:1.2 452.2:0.7 455.1:0.5 456.9:0.5 459.3:0.7 |
| | | | | | 460.3:1 461.3:1.9 462.3:0.7 463.3:1.9 464.1:0.7 |
| | | | | | 465.2:0.5 466.2:0.4 471.2:0.5 472.1:0.5 472.9:0.6 |
| | | | | | 475.1:0.9 477.3:0.5 478.3:0.8 479:1 480.2:0.8 481.2:19.9 |
| | | | | | 482.3:4.9 482.9:0.5 484.9:0.8 485.9:0.5 487:0.7 489.8:1 |
| | | | | | 492:0.9 493.2:18.1 494.3:4.7 494.9:0.6 495.4:0.9 |
| | | | | | 496.1:1.4 496.9:7.2 497.9:1.2 500.5:1 501:0.9 502:0.8 |
| | | | | | 503:0.7 504.1:0.5 505.2:2.4 506.2:0.8 508.4:0.5 509:1.5 |
| | | | | | 509.8:1.8 510.4:5.9 511.2:86.5 512.3:24.2 513.6:0.7 |
| | | | | | 514.5:1.8 515.5:1 516.4:0.5 517.7:0.9 518.2:1.2 |
| | | | | | 519.1:1.6 519.8:0.7 520.4:0.6 521:0.7 522.1:3.4 |
| | | | | | 523.2:100 524.3:26.5 525.2:1.1 526:0.7 527.2:0.7 |
| | | | | | 527.9:0.7 529:0.5 531.7:0.8 542.8:0.6 578.8:0.7 |
| | | | | | 612.9:0.6 648.9:0.9 684.8:0.5 718.7:0.9 766.7:0.5 |
| | | | | | 824.8:0.5 860.7:0.4 1018.5:0.7 1019.9:0.5 |
| Unknown - 11564 | 201 | 32881 | 177.1 | 1.2 | 55.3:0.7 57.2:5.8 59.2:0.8 71.2:0.8 73.1:13.3 74.2:1.3 |
| | | | | | 75.1:37.3 76.1:1.3 81.1:0.8 83.1:4.8 85.1:100 86.2:4.2 |
| | | | | | 87.1:0.8 89.1:1.2 99.1:0.9 100.1:1.1 101.2:1.5 105.1:0.8 |
| | | | | | 111.2:2.5 113.1:1.5 114:0.8 115.1:5.8 116.2:0.8 |
| | | | | | 117.1:1.1 121:0.7 126.2:0.8 129.1:11.5 130.1:0.8 |
| | | | | | 131.1:1.2 132.1:1.1 133.1:6.9 134.2:1.3 135.2:1.2 |
| | | | | | 136.1:7.9 143.1:1.1 144.8:1.1 147.1:0.9 148.2:1.9 |
| | | | | | 149.2:14.8 150.2:1.9 157.9:1.2 159.1:59.5 160.1:4.4 |
| | | | | | 163.1:1.1 177.1:4178.1:1.5 |
| Unknown - 11880 | 201 | 33225 | 537.4 | 5.44 | 213.3:0.1 237.3:0.1 239.3:0.3 254.4:0.1 255.3:0.2 |
| | | | | | 257.3:0.2 259.3:0.4 263.3:0.2 277.3:0.2 279.4:0.3 |
| | | | | | 280.4:0.3 281.4:0.1 295.4:0.2 296.6:0.2 297.4:0.7 |
| | | | | | 298.4:0.2 299.3:0.5 300.3:0.6 301.4:0.3 311.4:0.1 |
| | | | | | 313.4:0.6 314.4:0.2 315.3:3.8 316.4:0.8 333.3:2.2 |
| | | | | | 334.4:0.5 359.4:0.2 363.3:0.1 373.4:0.1 377.4:0.4 |
| | | | | | 378.4:0.1 391.5:0.2 395.4:0.2 399.4:0.2 405.4:0.7 |
| | | | | | 406.4:0.2 409.5:0.2 417.4:0.5 418.4:0.2 421.5:0.2 |
| | | | | | 439.5:0.1 457.4:3.4 458.5:1.3 465.5:0.2 473.6:0.3 |
| | | | | | 474.6:0.8 475.4:12.8 476.5:4.6 483.4:0.5 484.5:0.2 |
| | | | | | 491.5:0.2 492.5:0.2 493.4:2.2 494.5:0.7 501.4:5.5 |
| | | | | | 502.5:2 504.4:0.1 505.4:0.2 506.5:0.3 507.5:0.1 |
| | | | | | 517.6:0.6 519.4:100 520.3:31.5 521.1:0.2 |
| Unknown - 12846 | 201 | 34529 | 481.3 | 4.17 | 157.1:4.5 175.1:2.7 287.3:2.3 303.3:2.5 305.3:9.7 |
| | | | | | 306.3:1.7 317.3:1.3 333.3:1.7 334.3:0.5 347.3:10.8 |
| | | | | | 348.3:2.2 355.3:0.7 359.3:16.8 360.3:4 361.3:1.1 |
| | | | | | 363.3:5.2 364.3:1.3 373.3:2.9 375.3:4 376.3:1 383.3:0.7 |
| | | | | | 384.3:1.5 387.3:0.9 401.3:6.6 402.3:2.4 405.3:2.3 |
| | | | | | 406.4:0.7 419.3:1.1 421.3:9.5 422.3:1.9 435.3:1.1 |
| | | | | | 449.1:1.7 463.2:100 464.3:25 465.3:0.8 472.1:1.3 |
| Unknown - 17703 - | 201 | 40800 | 479.3 | 4.26 | 157:1.8 175.1:2.4 231.2:1.2 2312:0.6 275.2:0.4 |
| retired for 11- | | | | | 285.2:2.5 288.3:3.4 301.2:0.7 303.2:68 304.3:11.4 |
| ketoetiocholanolone | | | | | 3113:0.4 313.3:0.6 315.3:1.5 316.2:0.7 329.3:0.9 |
| glucuronide | | | | | 330.3:1.4 331.3:2.8 332.2:0.8 339.3:0.6 343.3:0.9 |
| | | | | | 345.3:10.7 346.3:1.9 357.3:2.6 358.3:0.7 361.2:22.9 |
| | | | | | 362.3:3.9 371.3:0.9 373.3:2.9 382.4:1.2 385.3:1.3 |
| | | | | | 386.2:0.6 399.3:2.5 400.3:1.6 402.3:3.6 403.3:2.6 |
| | | | | | 417.3:5 419.3:14.6 420.3:3.4 461.2:100 462.3:21.8 |
| Unknown - 18887 | 201 | 42272 | 328.2 | 2.17 | 104:4.1 127.1:0.2 128:0.4 180.2:0.2 183.2:0.2 197.2:0.5 |
| | | | | | 205.2:0.2 223.1:15.4 236.2:0.3 237.2:0.5 241.1:53.3 |
| | | | | | 2542:1.4 266.2:25.6 267.2:0.4 280.2:2.8 2842:3.9 |
| | | | | | 298.1:100 299.2:1.3 310.2:113 |
| Unknown - 18914 | 201 | 42299 | 266.9 | 4.43 | 140.9:0.3 194.1:0.8 195.1:0.6205:0.6 221:1.3 222.8:100 |
| | | | | | 223.9:5.8 247.9:1 248.9:14.2 249.9:1 265.9:3.4 266.9:3.3 |

Quant notes the molecular weight.
Biochemical name within the Metabolon database as well as the platform used for compound detection, the associated retention time (RT), the quant mass of the standard (Quant), and the MS/MS fragmentation of the quant ion coupled with the percent of the predominant peak (SPECTRA, frag:percent; for example 114.2:0.2 and 131.1:100 would indicate that 131.1 was the predominant mass of the MS/MS fragment and as the largest peak is designated as 100%. Mass 114.2 was detected as 0.2% of the MS/MS fragment in relation to peak 131.1).

TABLE 11
Models for estimating GFR from different sets of metabolites
Top 10 metabolites by rank of the correlation with average mGFR:

| | | | | | |
|---|---|---|---|---|---|
| Source | SS | df | MS | Number of obs = | 188 |
| Model | 29.6773957 | 10 | 2.96773957 | F(10, 177) = | 146.83 |
| Residual | 3.57753993 | 177 | .02021209 | Prob > F = | 0.0000 |
| Total | 33.2549356 | 187 | .17783388 | R-squared = | 0.8924 |
| | | | | Adj R-squared = | 0.8863 |
| | | | | Root MSE = | .14217 |

| loggfr_avg | Coef. | Std. Err. | t | P > \|t\| | [95% Conf. Interval] | | | rank |
|---|---|---|---|---|---|---|---|---|
| logbio_545 | −.2793396 | .0750019 | −3.72 | 0.000 | −.4273527 | −.1313265 | X-11564 | 1 |
| logbio_186 | −.3051049 | .066688 | −4.58 | 0.000 | −.4367109 | −.1734989 | C-glycosyltryptophan* | 2 |
| logbio_435 | −.1378877 | .0511305 | −2.70 | 0.008 | −.2387915 | −.0369839 | pseudouridine | 3 |
| logbio_746 | −.1971182 | .0760651 | −2.59 | 0.010 | −.3472295 | −.0470069 | X-17299 | 4 |
| logbio_374 | .0182053 | .0572352 | 0.32 | 0.751 | −.094746 | .1311565 | N-acetylthreonine | 5 |
| logbio_373 | −.0849153 | .0493913 | −1.72 | 0.087 | −.182387 | .0125564 | N-acetylserine | 6 |
| logbio_241 | −.0681421 | .0592983 | −1.15 | 0.252 | −.1851648 | .0438807 | erythritol | 7 |
| logbio_161 | −.0082856 | .0483569 | −0.17 | 0.864 | −.1037158 | .0871446 | arabitol | 8 |
| logbio_499 | −.0584699 | .045993 | −1.27 | 0.205 | −.1492352 | .0322954 | urea | 9 |
| logbio_714 | .0805344 | .0689427 | 1.17 | 0.244 | −.0555211 | .21659 | X-16394 | 10 |
| _cons | 3.848483 | .0118566 | 324.59 | 0.000 | 3.825085 | 3.871881 | | |

Best 6 by stepwise regression (p-value for entry 0.05, exit 0.01)

| | | | | | |
|---|---|---|---|---|---|
| Source | SS | df | MS | Number of obs = | 188 |
| Model | 29.7546488 | 6 | 4.95910814 | F(6, 181) = | 256.44 |
| Residual | 3.50028682 | 181 | .019338601 | Prob > F | 0.0000 |
| Total | 33.2549356 | 187 | .17783388 | R-squared = | 0.8947 |
| | | | | Adj R-squared = | 0.8913 |
| | | | | Root MSE | .13906 |

| loggfr_avg | Coef. | Std. Err. | t | P > \|t\| | [95% Conf. Interval] | | | rank |
|---|---|---|---|---|---|---|---|---|
| logbio_545 | −.3342452 | .0641511 | −5.21 | 0.000 | −.4608255 | −.207665 | X-11564 | 1 |
| logbio_186 | −.3359736 | .0605076 | −5.55 | 0.000 | −.4553645 | −.2165827 | C-glycosyltryptophan* | 2 |
| logbio_399 | −.0544081 | .0170302 | −3.19 | 0.002 | −.0880115 | −.0208048 | p-cresol sulfate | 37 |
| logbio_359 | −.1125838 | .0368361 | −3.06 | 0.003 | −.1852673 | −.0399004 | myo-inositol | 14 |
| logbio_514 | −.0622925 | .0225565 | −2.76 | 0.006 | −.1068 | −.0177851 | X-02249 | 48 |
| logbio_435 | −.132522 | .0488687 | −2.71 | 0.007 | −.2289477 | −.0360963 | pseudouridine | 3 |
| _cons | 3.848898 | .0112664 | 341.63 | 0.000 | 3.826668 | 3.871128 | | |

Best 5 Considering Jaffe Cr stepwise regression (p-value for entry 0.05, exit 0.01)

| | | | | | |
|---|---|---|---|---|---|
| Source | SS | df | MS | Number of obs = | 188 |
| Model | 29.8047582 | 6 | 4.9674597 | F(6, 181) = | 260.6 |
| Residual | 3.45017744 | 181 | .019061754 | Prob > F = | 0.0000 |
| Total | 33.2549356 | 187 | .17783388 | R-squared = | 0.8963 |
| | | | | Adj R-squared = | 0.8928 |
| | | | | Root MSE = | .13806 |

| loggfr_avg | Coef. | Std. Err. | t | P > \|t\| | [95% Conf. Interval] | | | |
|---|---|---|---|---|---|---|---|---|
| logbio_545 | −.3213439 | .0690627 | −4.65 | 0.000 | −.4576156 | −.1850723 | X-11564 | 1 |
| logbio_186 | −.4067093 | .0553595 | −7.35 | 0.000 | −.5159422 | −.2974763 | C-glycosyltryptophan* | 2 |
| logscr | −.1725016 | .0576152 | −2.99 | 0.003 | −.2861854 | −.0588178 | | |
| logbio_334 | .2105805 | .0592975 | 3.55 | 0.000 | .0935772 | .3275838 | leucine | 750 |
| logbio_26 | −.0661812 | .0191195 | −3.46 | 0.001 | −.1039069 | −.0284555 | 1-methylhistidine | 22 |
| logbio_28 | .0419139 | .0150977 | 2.78 | 0.006 | .0121238 | .071704 | 1- | 735 |
| | | | | | | | myristoylglycerophos- | |
| | | | | | | | phocholine (14:0) | |
| _cons | 3.947145 | .0315138 | 125.25 | 0.000 | 3.884963 | 4.009327 | | |

Best 5 Considering Jaffe Cr & CysC stepwise regression (p-value for entry 0.05, exit 0.01)

| | | | | | |
|---|---|---|---|---|---|
| Source | SS | df | MS | Number of obs = | 188 |
| Model | 30.0081638 | 6 | 5.00136063 | F(6, 181) = | 278.81 |
| Residual | 3.24677189 | 181 | .017937966 | Prob > F = | 0.0000 |
| Total | 33.2549356 | 187 | .17783388 | R-squared = | 0.9024 |
| | | | | Adj R-squared = | 0.8991 |
| | | | | Root MSE = | .13393 |

| loggfr_avg | Coef. | Std. Err. | t | P > \|t\| | [95% Conf. Interval] | | | |
|---|---|---|---|---|---|---|---|---|
| logcys | −.421111 | .0851197 | −4.95 | 0.000 | −.5890656 | −.2531564 | | |
| logbio_545 | −.2253853 | .0664313 | −3.39 | 0.001 | −.3564646 | −.094306 | X-11564 | 1 |
| logbio_186 | −.2240287 | .0645688 | −3.47 | 0.001 | −.351433 | −.0966244 | C-glycosyltryptophan* | 2 |
| logbio_775 | −.0630226 | .0196714 | −3.20 | 0.002 | −.1018374 | −.0242078 | X-17703 | 32 |
| logbio_514 | −.0642893 | .021694 | −2.96 | 0.003 | −.1070949 | −.0214837 | X-02249 | 48 |
| logbio_359 | −.1041984 | .0355785 | −2.93 | 0.004 | −.1744004 | −.0339965 | myo-inositol | 14 |
| _cons | 4.038692 | .0385863 | 104.67 | 0.000 | 3.962555 | 4.114829 | | |

Best 7 with age and sex by stepwise regression (p-value for entry 0.05, exit 0.01)

| | | | | | |
|---|---|---|---|---|---|
| Source | SS | df | MS | Number of obs = | 188 |
| Model | 30.2637024 | 9 | 3.3626336 | F(9, 178) = | 200.1 |
| Residual | 2.99123322 | 178 | .016804681 | Prob > F = | 0.0000 |
| Total | 33.2549356 | 187 | .17783388 | R-squared = | 0.9101 |
| | | | | Adj R-squared = | 0.9055 |
| | | | | Root MSE = | .12963 |

| loggfr_avg | Coef. | Std. Err. | t | P > \|t\| | [95% Conf. Interval] | | | |
|---|---|---|---|---|---|---|---|---|
| sex | −.0641232 | .0222895 | −2.88 | 0.005 | −.1081088 | −.0201375 | | |
| logbio_545 | −.3556788 | .0637406 | −5.58 | 0.000 | −.4814632 | −.2298944 | X-11564 | 1 |
| logbio_186 | −.1985949 | .0612066 | −3.24 | 0.001 | −.3193788 | −.0778111 | C-glycosyltryptophan* | 2 |
| logbio_746 | −.1715388 | .0406973 | −4.21 | 0.000 | −.2518502 | −.0912275 | X-17299 | 4 |
| logbio_373 | −.1117963 | .0417506 | −2.68 | 0.008 | −.1941862 | −.0294064 | N-acetylserine | 6 |
| logbio_435 | −.1365187 | .0458425 | −2.98 | 0.003 | −.2269833 | −.0460541 | pseudouridine | 3 |
| age | −.0042299 | .0010683 | −3.96 | 0.000 | −.006338 | −.0021218 | | |
| logbio_179 | .1190674 | .0359501 | 3.31 | 0.001 | .0481242 | .1900107 | betaine | 771 |
| logbio_64 | .0671294 | .0227812 | 2.95 | 0.004 | .0221735 | .1120854 | 2-hydroxybutyrate | 768 |
| | | | | | | | (AHB) | |
| _cons | 4.194703 | .0687437 | 61.02 | 0.000 | 4.059046 | 4.330361 | | |

Best 6 considering Cr with age and sex by stepwise regression (p-value for entry 0.05, exit 0.01)

| | | | | | |
|---|---|---|---|---|---|
| Source | SS | df | MS | Number of obs = | 188 |
| Model | 30.4941791 | 9 | 3.38824212 | F(9, 178) = | 218.46 |
| Residual | 2.76075654 | 178 | .015509868 | Prob > F = | 0.0000 |
| Total | 33.2549356 | 187 | .17783388 | R-squared = | 0.9170 |
| | | | | Adj R-squared = | 0.9128 |
| | | | | Root MSE = | .12454 |

| loggfr_avg | Coef. | Std. Err. | t | P > \|t\| | [95% Conf. Interval] | | | |
|---|---|---|---|---|---|---|---|---|
| sex | −.1231985 | .0223375 | −5.52 | 0.000 | −.1672789 | −.079118 | | |
| logbio_545 | −.3393453 | .0641557 | −5.29 | 0.000 | −.4659489 | −.2127417 | X-11564 | 1 |
| logbio_186 | −.2988977 | .0537978 | −5.56 | 0.000 | −.4050613 | −.192734 | C-glycosyltryptophan* | 2 |
| logscr | −.3220039 | .0580755 | −5.54 | 0.000 | −.436609 | −.2073989 | | |
| age | −.0033352 | .0010187 | −3.27 | 0.001 | −.0053454 | −.0013249 | | |
| logbio_26 | −.0557669 | .0171424 | −3.25 | 0.001 | −.0895954 | −.0219384 | 1-methylhistidine | 22 |
| logbio_64 | .0779415 | .0217686 | 3.58 | 0.000 | .0349839 | .1208992 | 2-hydroxybutyrate | 768 |
| | | | | | | | (AHB) | |
| logbio_28 | .0541034 | .0138153 | 3.92 | 0.000 | .0268405 | .0813662 | 1- | 735 |
| | | | | | | | myristoylglycerophos- | |
| | | | | | | | phocholine (14:0) | |
| logbio_801 | −.0527723 | .0176463 | −2.99 | 0.003 | −.0875951 | −.0179494 | X-18914 | 733 |
| _cons | 4.370895 | .0825673 | 52.94 | 0.000 | 4.207959 | 4.533832 | | |

Best 3 considering Jaffe Cr & Cys with age and sex by stepwise regression (p-value for entry 0.05, exit 0.01)

| | | | | | |
|---|---|---|---|---|---|
| Source | SS | df | MS | Number of obs = | 188 |
| Model | 30.3465079 | 7 | 4.33521541 | F(7, 180) = | 268.3 |
| Residual | 2.90842779 | 180 | .016157932 | Prob > F = | 0.0000 |
| Total | 33.2549356 | 187 | .17783388 | R-squared = | 0.9125 |
| | | | | Adj R-squared = | 0.9091 |
| | | | | Root MSE = | .12711 |

| loggfr_avg | Coef. | Std. Err. | t | P > \|t\| | [95% Conf. Interval] | | | |
|---|---|---|---|---|---|---|---|---|
| sex | −.117144 | .0230539 | −5.08 | 0.000 | −.1626347 | −.0716534 | | |
| logcys | −.3515307 | .0821922 | −4.28 | 0.000 | −.5137148 | −.1893465 | | |
| logscr | −.3087382 | .0590852 | −5.23 | 0.000 | −.425327 | −.1921495 | | |
| logbio_186 | −.1817934 | .0625769 | −2.91 | 0.004 | −.3052722 | −.0583147 | C-glycosyltryptophan* | 2 |
| age | −.0037233 | .0010197 | −3.65 | 0.000 | −.0057355 | −.0017111 | | |
| logbio_373 | −.1094776 | .0407618 | −2.69 | 0.008 | −.18991 | −.0290453 | N-acetylserine | 6 |
| logbio_545 | −.1850715 | .0695111 | −2.66 | 0.008 | −.3222329 | −.04791 | X-11564 | 1 |
| _cons | 4.553079 | .0871312 | 52.26 | 0.000 | 4.38115 | 4.725009 | | |

Best 14 by stepwise regression (p-value for entry 0.05, exit 0.10)

| | | | | | |
|---|---|---|---|---|---|
| Source | SS | df | MS | Number of obs = | 188 |
| Model | 30.6143528 | 14 | 2.18673948 | F(14, 173) = | 143.27 |
| Residual | 2.64058286 | 173 | .015263485 | Prob > F = | 0.0000 |
| Total | 33.2549356 | 187 | .17783388 | R-squared = | 0.9206 |
| | | | | Adj R-squared = | 0.9142 |
| | | | | Root MSE = | .12355 |

| loggfr_avg | Coef. | Std. Err. | t | P > \|t\| | [95% Conf. Interval] | | | |
|---|---|---|---|---|---|---|---|---|
| logbio_545 | −.2962129 | .0639477 | −4.63 | 0.000 | −.4224309 | −.1699948 | X-11564 | 1 |
| logbio_186 | −.2632994 | .0566509 | −4.65 | 0.000 | −.3751153 | −.1514835 | C-glycosyltryptophan* | 2 |
| logbio_399 | −.034826 | .0159617 | −2.18 | 0.030 | −.0663308 | −.0033212 | p-cresol sulfate | 37 |
| logbio_359 | −.0999066 | .0340143 | −2.94 | 0.004 | −.1670431 | −.0327701 | myo-inositol | 14 |
| logbio_514 | −.0621536 | .0206351 | −3.01 | 0.003 | −.1028826 | −.0214247 | X-02249 | 48 |
| logbio_576 | −.0801229 | .0263476 | −3.04 | 0.003 | −.1321271 | −.0281187 | X-12104 | 17 |
| logbio_363 | −.0383981 | .0202832 | −1.89 | 0.060 | −.0784325 | .0016364 | N-acetyl-1- | 30 |
| | | | | | | | methylhistidine* | |
| logbio_64 | .0924565 | .0219177 | 4.22 | 0.000 | .0491958 | .1357171 | 2-hydroxybutyrate | 768 |
| | | | | | | | (AHB) | |
| logbio_801 | −.0753593 | .017858 | −4.22 | 0.000 | −.1106069 | −.0401117 | X-18914 | 733 |
| logbio_565 | .0594912 | .0228072 | 2.61 | 0.010 | .0144751 | .1045074 | X-11880 | 763 |
| logbio_746 | −.2081663 | .0645693 | −3.22 | 0.002 | −.3356113 | −.0807213 | X-17299 | 4 |
| logbio_714 | .1094817 | .0586206 | 1.87 | 0.064 | −.0062221 | .2251854 | X-16394 | 10 |
| logbio_179 | .0794282 | .0341929 | 2.32 | 0.021 | .0119394 | .1469171 | betaine | 771 |
| logbio_28 | .0312802 | .0145216 | 2.15 | 0.033 | .0026178 | .0599426 | 1- | 735 |
| | | | | | | | myristoylglycerophos- | |
| | | | | | | | phocholine (14:0) | |
| _cons | 3.819721 | .011938 | 319.96 | 0.000 | 3.796158 | 3.843284 | | |

Best 15 with age and sex by stepwise regression (p-value for entry 0.05, exit 0.10)

| | | | | | |
|---|---|---|---|---|---|
| Source | SS | df | MS | Number of obs = | 188 |
| Model | 31.0489477 | 17 | 1.82640869 | F(17, 170) = | 140.75 |
| Residual | 2.20598799 | 170 | .0129764 | Prob > F = | 0.0000 |
| Total | 33.2549356 | 187 | .17783388 | R-squared = | 0.9337 |
| | | | | Adj R-squared = | 0.9270 |
| | | | | Root MSE = | .11391 |

| loggfr_avg | Coef. | Std. Err. | t | P > \|t\| | [95% Conf. Interval] | | | |
|---|---|---|---|---|---|---|---|---|
| sex | −.0640739 | .0222685 | −2.88 | 0.005 | −.1080323 | −.0201155 | | |
| logbio_545 | −.3716149 | .0604945 | −6.14 | 0.000 | −.4910321 | −.2521976 | X-11564 | 1 |
| logbio_186 | −.2454109 | .0546347 | −4.49 | 0.000 | −.3532608 | −.1375611 | C-glycosyltryptophan* | 2 |
| logbio_746 | −.1211033 | .0402948 | −3.01 | 0.003 | −.2006459 | −.0415608 | X-17299 | 4 |
| logbio_26 | −.0576107 | .016334 | −3.53 | 0.001 | −.0898543 | −.0253671 | 1-methylhistidine | 22 |
| logbio_435 | −.0951873 | .0420478 | −2.26 | 0.025 | −.1781904 | −.0121843 | pseudouridine | 3 |
| age | −.0049483 | .001 | −4.95 | 0.000 | −.0069223 | −.0029743 | | |
| logbio_179 | .1095119 | .0347806 | 3.15 | 0.002 | .0408545 | .1781693 | betaine | 771 |
| logbio_64 | .0893123 | .0218164 | 4.09 | 0.000 | .0462462 | .1323783 | 2-hydroxybutyrate | 768 |
| | | | | | | | (AHB) | |
| logbio_28 | .0467959 | .0132206 | 3.54 | 0.001 | .0206981 | .0728936 | 1- | 735 |
| | | | | | | | myristoylglycerophos- | |
| | | | | | | | phocholine (14:0) | |
| logbio_801 | −.0528736 | .0169833 | −3.11 | 0.002 | −.0863989 | −.0193483 | X-18914 | 733 |
| logbio_565 | .0613779 | .0214267 | 2.86 | 0.005 | .0190813 | .1036745 | X-11880 | 763 |
| logbio_514 | −.0775763 | .0208573 | −3.72 | 0.000 | −.1187489 | −.0364037 | X-02249 | 48 |
| logbio_525 | .1037361 | .0408685 | 2.54 | 0.012 | .0230611 | .1844112 | X-11423 | 11 |
| logbio_69 | −.081077 | .0282899 | −2.87 | 0.005 | −.1369217 | −.0252322 | 2-hydroxyisobutyrate | 25 |
| logbio_625 | .0532433 | .0222128 | 2.40 | 0.018 | .0093948 | .0970918 | X-12822 | 46 |
| logbio_214 | −.1340897 | .0640747 | −2.09 | 0.038 | −.2605743 | −.0076051 | creatinine | 13 |
| _cons | 4.218622 | .0655063 | 64.40 | 0.000 | 4.089311 | 4.347933 | | |

TABLE 12
Models for estimating GFR from different sets of metabolites - limited to KNOWN metabolites
Top 10 metabolites by rank of the correlation with average mGFR:

| | | | | | |
|---|---|---|---|---|---|
| Source | SS | df | MS | Number of obs = | 188 |
| Model | 29.3625599 | 10 | 2.93625599 | F(10, 177) = | 133.52 |
| Residual | 3.89237572 | 177 | .021990823 | Prob > F = | 0.0000 |
| Total | 33.2549356 | 187 | .17783388 | R-squared = | 0.8830 |
| | | | | Adj R-squared = | 0.8763 |
| | | | | Root MSE = | .14829 |

| loggfr_avg | Coef. | Std. Err. | t | P > \|t\| | [95% Conf. Interval] | | | rank |
|---|---|---|---|---|---|---|---|---|
| logbio_186 | −.3727788 | .0682989 | −5.46 | 0.000 | −.5075638 | −.2379938 | C-glycosyltryptophan* | 1 |
| logbio_435 | −.1794846 | .05238 | −3.43 | 0.001 | −.2828543 | −.0761148 | pseudouridine | 2 |
| logbio_374 | .0031172 | .0561254 | 0.06 | 0.956 | −.1076438 | .1138782 | N-acetylthreonine | 3 |
| logbio_373 | −.0743013 | .0518694 | −1.43 | 0.154 | −.1766633 | .0280608 | N-acetylserine | 4 |
| logbio_241 | −.016929 | .0778251 | −0.22 | 0.828 | −.1705135 | .1366556 | erythritol | 5 |
| logbio_161 | .0048379 | .0502224 | 0.10 | 0.923 | −.0942739 | .1039497 | arabitol | 6 |
| logbio_499 | −.1285772 | .0461999 | −2.78 | 0.006 | −.2197507 | −.0374037 | urea | 7 |
| logbio_242 | −.110935 | .0725427 | −1.53 | 0.128 | −.2540948 | .0322248 | erythronate* | 8 |
| logbio_214 | −.1692218 | .0640916 | −2.64 | 0.009 | −.2957037 | −.0427398 | creatinine | 9 |
| logbio_359 | −.079701 | .0459336 | −1.74 | 0.084 | −.170349 | .0109471 | myo-inositol | 10 |
| _cons | 3.839496 | .011717 | 327.69 | 0.000 | 3.816373 | 3.862619 | | |

Best 5 by stepwise regression (p-value for entry 0.05, exit 0.01)

| | | | | | |
|---|---|---|---|---|---|
| Source | SS | df | MS | Number of obs = | 188 |
| Model | 29.2433588 | 5 | 5.84867176 | F(5, 182) = | 265.35 |
| Residual | 4.01157687 | 182 | .022041631 | Prob > F = | 0.0000 |
| Total | 33.2549356 | 187 | .17783388 | R-squared = | 0.8794 |
| | | | | Adj R-squared = | 0.8761 |
| | | | | Root MSE = | .14846 |

| loggfr_avg | Coef. | Std. Err. | t | P > \|t\| | [95% Conf. Interval] | | | |
|---|---|---|---|---|---|---|---|---|
| logbio_186 | −.4516763 | .0572056 | −7.90 | 0.000 | −.5645477 | −.3388048 | C-glycosyltryptophan* | 2 |
| logbio_359 | −.1938124 | .0362057 | −5.35 | 0.000 | −.2652493 | −.1223754 | myo-inositol | 14 |
| logbio_435 | −.2002827 | .0504042 | −3.97 | 0.000 | −.2997344 | −.100831 | pseudouridine | 3 |
| logbio_363 | −.0745704 | .0226755 | −3.29 | 0.001 | −.119311 | −.0298298 | N-acetyl-1- | 30 |
| | | | | | | | methylhistidine* | |
| logbio_411 | −.0530628 | .017097 | −3.10 | 0.002 | −.0867966 | −.0193289 | phenylacetylglutamine | 65 |
| _cons | 3.836586 | .0110301 | 347.83 | 0.000 | 3.814822 | 3.858349 | | |

Best 4 Considering Jaffe Cr by stepwise regression (p-value for entry 0.05, exit 0.01)

| | | | | | |
|---|---|---|---|---|---|
| Source | SS | df | MS | Number of obs = | 188 |
| Model | 29.480897 | 5 | 5.8961794 | F(5, 182) = | 284.34 |
| Residual | 3.77403864 | 182 | .020736476 | Prob > F = | 0.0000 |
| Total | 33.2549356 | 187 | .17783388 | R-squared = | 0.8865 |
| | | | | Adj R-squared = | 0.8834 |
| | | | | Root MSE = | .144 |

| loggfr_avg | Coef. | Std. Err. | t | P > \|t\| | [95% Conf. Interval] | | | |
|---|---|---|---|---|---|---|---|---|
| logbio_186 | −.4404718 | .0545791 | −8.07 | 0.000 | −.548161 | −.3327825 | C-glycosyltryptophan* | 2 |
| logscr | −.2516286 | .0525246 | −4.79 | 0.000 | −.355264 | −.1479931 | | |
| logbio_435 | −.1727211 | .0495595 | −3.49 | 0.001 | −.2705061 | −.0749361 | pseudouridine | 3 |
| logbio_359 | −.1344265 | .037874 | −3.55 | 0.000 | −.2091551 | −.059698 | myo-inositol | 14 |
| logbio_411 | −.0507358 | .016597 | −3.06 | 0.003 | −.083483 | −.0179886 | phenylacetylglutamine | 65 |
| _cons | 3.973754 | .0308436 | 128.84 | 0.000 | 3.912897 | 4.034611 | | |

Best 4 Considering Jaffe Cr & CysC by stepwise regression (p-value for entry 0.05, exit 0.01)

| | | | | | |
|---|---|---|---|---|---|
| Source | SS | df | MS | Number of obs = | 188 |
| Model | 30.0051216 | 6 | 5.0008536 | F(6, 181) = | 278.53 |
| Residual | 3.24981406 | 181 | .017954774 | Prob > F = | 0.0000 |
| Total | 33.2549356 | 187 | .17783388 | R-squared = | 0.9023 |
| | | | | Adj R-squared = | 0.8990 |
| | | | | Root MSE = | .134 |

| loggfr_avg | Coef. | Std. Err. | t | P > \|t\| | [95% Conf. Interval] | | | |
|---|---|---|---|---|---|---|---|---|
| logcys | −.4932587 | .0813172 | −6.07 | 0.000 | −.6537103 | −.332807 | | |
| logbio_186 | −.2744222 | .0612087 | −4.48 | 0.000 | −.3951966 | −.1536479 | C-glycosyltryptophan* | 2 |
| logscr | −.1880854 | .050578 | −3.72 | 0.000 | −.2878838 | −.0882869 | | |
| logbio_267 | .1034787 | .0322551 | 3.21 | 0.002 | .0398344 | .167123 | glutamate | 720 |
| logbio_359 | −.110188 | .0355551 | −3.10 | 0.002 | −.1803438 | −.0400321 | myo-inositol | 14 |
| logbio_411 | −.0431129 | .015509 | −2.78 | 0.006 | −.0737147 | −.0125112 | phenylacetylglutamine | 65 |
| _cons | 4.159065 | .0395046 | 105.28 | 0.000 | 4.081117 | 4.237014 | | |

Best 7 with age and sex by stepwise regression (p-value for entry 0.05, exit 0.01)

| | | | | | |
|---|---|---|---|---|---|
| Source | SS | df | MS | Number of obs = | 188 |
| Model | 29.7276535 | 8 | 3.71595669 | F(8, 179) = | 188.57 |
| Residual | 3.5272821 | 179 | .019705487 | Prob > F = | 0.0000 |
| Total | 33.2549356 | 187 | .17783388 | R-squared = | 0.8939 |
| | | | | Adj R-squared = | 0.8892 |
| | | | | Root MSE = | .14038 |

| loggfr_avg | Coef. | Std. Err. | t | P > \|t\| | [95% Conf. Interval] | | | |
|---|---|---|---|---|---|---|---|---|
| sex | −.0997563 | .0250508 | −3.98 | 0.000 | −.1491892 | −.0503233 | | |
| logbio_186 | −.3848388 | .0566999 | −6.79 | 0.000 | −.4967251 | −.2729525 | C-glycosyltryptophan* | 2 |
| logbio_359 | −.1435196 | .0361156 | −3.97 | 0.000 | −.2147868 | −.0722524 | myo-inositol | 14 |
| logbio_435 | −.1644943 | .0482939 | −3.41 | 0.001 | −.2597929 | −.0691957 | pseudouridine | 3 |
| logbio_214 | −.2481113 | .0676176 | −3.67 | 0.000 | −.3815414 | −.1146812 | creatinine | 13 |
| logbio_26 | −.0591928 | .0195833 | −3.02 | 0.003 | −.0978367 | −.0205489 | 1-methylhistidine | 22 |
| logbio_117 | −.0309718 | .0112772 | −2.75 | 0.007 | −.0532252 | −.0087183 | | |
| logbio_363 | −.0607034 | .0221166 | −2.74 | 0.007 | −.1043461 | −.0170606 | | |
| _cons | 3.931865 | .0353317 | 111.28 | 0.000 | 3.862145 | 4.001586 | | |

Best 3 considering Cr with age and sex by stepwise regression (p-value for entry 0.05, exit 0.01)

| | | | | | |
|---|---|---|---|---|---|
| Source | SS | df | MS | Number of obs = | 188 |
| Model | 29.9281464 | 6 | 4.9880244 | F(6, 181) = | 271.38 |
| Residual | 3.32678927 | 181 | .018380051 | Prob > F = | 0.0000 |
| Total | 33.2549356 | 187 | .17783388 | R-squared = | 0.9000 |
| | | | | Adj R-squared = | 0.8966 |
| | | | | Root MSE = | .13557 |

| loggfr_avg | Coef. | Std. Err. | t | P > \|t\| | [95% Conf. Interval] | | | |
|---|---|---|---|---|---|---|---|---|
| sex | −.1348889 | .0242553 | −5.56 | 0.000 | −.1827485 | −.0870293 | | |
| logbio_186 | −.3615767 | .0550829 | −6.56 | 0.000 | −.4702639 | −.2528896 | C-glycosyltryptophan* | 2 |
| logscr | −.4472268 | .0529115 | −8.45 | 0.000 | −.5516295 | −.342824 | | |
| logbio_373 | −.1482477 | .0426217 | −3.48 | 0.001 | −.2323471 | −.0641483 | N-acetylserine | 6 |
| age | −.0034491 | .0010837 | −3.18 | 0.002 | −.0055874 | −.0013108 | | |
| logbio_435 | −.1420244 | .0474717 | −2.99 | 0.003 | −.2356936 | −.0483552 | pseudouridine | 3 |
| _cons | 4.464948 | .0868307 | 51.42 | 0.000 | 4.293618 | 4.636279 | | |

Best 2 considering Cr + Cys with age and sex by stepwise regression (p-value for entry 0.05, exit 0.01)

| | | | | | |
|---|---|---|---|---|---|
| Source | SS | df | MS | Number of obs = | 188 |
| Model | 30.231968 | 6 | 5.03866134 | F(6, 181) = | 301.69 |
| Residual | 3.02296761 | 181 | .016701479 | Prob > F = | 0.0000 |
| Total | 33.2549356 | 187 | .17783388 | R-squared = | 0.9091 |
| | | | | Adj R-squared = | 0.9061 |
| | | | | Root MSE = | .12923 |

| loggfr_avg | Coef. | Std. Err. | t | P > \|t\| | [95% Conf. Interval] | | | |
|---|---|---|---|---|---|---|---|---|
| sex | −.1266131 | .0231579 | −5.47 | 0.000 | −.1723072 | −.080919 | | |
| logcys | −.4201715 | .0793461 | −5.30 | 0.000 | −.5767338 | −.2636092 | | |
| logscr | −.3854155 | .0524519 | −7.35 | 0.000 | −.4889113 | −.2819196 | | |
| logbio_186 | −.229729 | .0609307 | −3.77 | 0.000 | −.3499548 | −.1095031 | C-glycosyltryptophan* | 2 |
| age | −.0034769 | .0010325 | −3.37 | 0.001 | −.0055141 | −.0014397 | | |
| logbio_373 | −.1280338 | .0408314 | −3.14 | 0.002 | −.2086006 | −.047467 | N-acetylserine | 6 |
| _cons | 4.612864 | .0855924 | 53.89 | 0.000 | 4.443976 | 4.781751 | | |

Best 14 by stepwise regression (p-value for entry 0.05, exit 0.10)

| | | | | | |
|---|---|---|---|---|---|
| Source | SS | df | MS | Number of obs = | 188 |
| Model | 30.3573381 | 14 | 2.1683813 | F(14, 173) = | 129.46 |
| Residual | 2.89759751 | 173 | .016749119 | Prob > F = | 0.0000 |
| Total | 33.2549356 | 187 | .17783388 | R-squared = | 0.9129 |
| | | | | Adj R-squared = | 0.9058 |
| | | | | Root MSE = | .12942 |

| loggfr_avg | Coef. | Std. Err. | t | P > \|t\| | [95% Conf. Interval] | | | |
|---|---|---|---|---|---|---|---|---|
| logbio_186 | −.2657648 | .0649383 | −4.09 | 0.000 | −.3939382 | −.1375914 | C-glycosyltryptophan* | 2 |
| logbio_359 | −.10777 | .0383701 | −2.81 | 0.006 | −.1835039 | −.0320361 | myo-inositol | 14 |
| logbio_435 | −.1091854 | .0465494 | −2.35 | 0.020 | −.2010633 | −.0173075 | pseudouridine | 3 |
| logbio_363 | −.0592134 | .0205725 | −2.88 | 0.005 | −.0998189 | −.0186079 | N-acetyl-1- | 30 |
| | | | | | | | methylhistidine* | |
| logbio_267 | .0892623 | .0321947 | 2.77 | 0.006 | .0257173 | .1528073 | glutamate | 720 |
| logbio_117 | −.033102 | .0107415 | −3.08 | 0.002 | −.0543034 | −.0119007 | 4-acetylphenol sulfate | 67 |
| logbio_179 | .0947328 | .0344893 | 2.75 | 0.007 | .0266587 | .1628068 | betaine | 771 |
| logbio_114 | −.1365022 | .0427036 | −3.20 | 0.002 | −.2207893 | −.052215 | 4-acetamidobutanoate | 20 |
| logbio_388 | .0947271 | .025122 | 3.77 | 0.000 | .045142 | .1443122 | nonadecanoate (19:0) | 713 |
| logbio_276 | .0696652 | .0318185 | 2.19 | 0.030 | .0068628 | .1324676 | glycerophosphorylcholine | 775 |
| | | | | | | | (GPC) | |
| logbio_242 | −.2273255 | .0540391 | −4.21 | 0.000 | −.3339862 | −.1206647 | erythronate* | 12 |
| logbio_162 | .0909324 | .0307211 | 2.96 | 0.004 | .030296 | .1515688 | arabonate | 23 |
| logbio_143 | −.1087806 | .0354705 | −3.07 | 0.003 | −.1787912 | −.0387699 | acetylcarnitine | 74 |
| logbio_153 | .0434747 | .0214437 | 2.03 | 0.044 | .0011497 | .0857996 | alpha-hydroxyisocaproate | 751 |
| _cons | 3.827182 | .0170292 | 224.74 | 0.000 | 3.79357 | 3.860794 | | |

Best 14 with age and sex by stepwise regression (p-value for entry 0.05, exit 0.10)

| | | | | | |
|---|---|---|---|---|---|
| Source | SS | df | MS | Number of obs = | 188 |
| Model | 30.5388215 | 16 | 1.90867635 | F(16, 171) = | 120.17 |
| Residual | 2.71611412 | 171 | .015883708 | Prob > F = | 0.0000 |
| Total | 33.2549356 | 187 | .17783388 | R-squared = | 0.9183 |
| | | | | Adj R-squared = | 0.9107 |
| | | | | Root MSE = | .12603 |

| loggfr_avg | Coef. | Std. Err. | t | P > \|t\| | [95% Conf. Interval] | | | |
|---|---|---|---|---|---|---|---|---|
| sex | −.1020999 | .0231958 | −4.40 | 0.000 | −.1478869 | −.0563129 | | |
| logbio_186 | −.2494251 | .0636979 | −3.92 | 0.000 | −.3751605 | −.1236897 | C-glycosyltryptophan* | 2 |
| logbio_359 | −.1385033 | .0377107 | −3.67 | 0.000 | −.2129418 | −.0640649 | myo-inositol | 14 |
| logbio_435 | −.1166252 | .0455107 | −2.56 | 0.011 | −.2064603 | −.0267902 | pseudouridine | 3 |
| logbio_214 | −.2151104 | .0628079 | −3.42 | 0.001 | −.3390891 | −.0911317 | creatinine | 13 |
| logbio_26 | −.0424486 | .0180133 | −2.36 | 0.020 | −.0780056 | −.0068916 | 1-methylhistidine | 22 |
| logbio_117 | −.0313871 | .0103351 | −3.04 | 0.003 | −.0517879 | −.0109863 | 4-acetylphenol sulfate | 67 |
| logbio_363 | −.0682415 | .0201418 | −3.39 | 0.001 | −.1080002 | −.0284829 | N-acetyl-1- | 30 |
| | | | | | | | methylhistidine* | |
| age | −.0024581 | .0010082 | −2.44 | 0.016 | −.0044483 | −.0004679 | | |
| logbio_114 | −.1000038 | .0413284 | −2.42 | 0.017 | −.1815832 | −.0184243 | 4-acetamidobutanoate | 20 |
| logbio_388 | .0619684 | .0237429 | 2.61 | 0.010 | .0151015 | .1088353 | nonadecanoate (19:0) | 713 |
| logbio_276 | .0881 | .0286987 | 3.07 | 0.002 | .0314506 | .1447493 | glycerophosphorylcholine | 775 |
| logbio_242 | −.1774798 | .0526769 | −3.37 | 0.001 | −.2814606 | −.073499 | erythronate* | 12 |
| logbio_480 | .0673966 | .0263307 | 2.56 | 0.011 | .0154215 | .1193716 | threitol | 34 |
| logbio_366 | −.1468202 | .0577934 | −2.54 | 0.012 | −.2609005 | −.0327399 | N-acetylalanine | 18 |
| logbio_162 | .0608557 | .0301739 | 2.02 | 0.045 | .0012944 | .1204169 | arabonate | 23 |
| _cons | 4.085822 | .0685151 | 59.63 | 0.000 | 3.950578 | 4.221066 | | |

TABLE 13
List of All Metabolites Ranked by Their Correlation with MGFR

| | Correlation with average mGFR | | | | | | | |
|---|---|---|---|---|---|---|---|---|
| | | | partial r | | | | | |
| | | | with | Correlation with Age | | Correlation with Sex | | |
| Metabolite # | r | p-value | creatinine | r | p-value | r | p-value | Biochemical Name |
| bio_545 | −0.808 | 0 | −0.443 | −0.047 | 0.527 | 0.039 | 0.595 | X-11564 |
| bio_186 | −0.787 | 0 | −0.446 | 0.020 | 0.788 | −0.008 | 0.909 | C-glycosyltryptophan* |
| bio_435 | −0.774 | 0 | −0.413 | −0.040 | 0.587 | 0.004 | 0.953 | pseudouridine |
| bio_746 | −0.768 | 0 | −0.329 | −0.026 | 0.722 | 0.128 | 0.081 | X-17299 |
| bio_374 | −0.766 | 0 | −0.501 | −0.042 | 0.566 | 0.062 | 0.396 | N-acetylthreonine |
| bio_373 | −0.758 | 0 | −0.385 | −0.011 | 0.879 | 0.148 | 0.043 | N-acetylserine |
| bio_241 | −0.758 | 0 | −0.371 | 0.071 | 0.335 | 0.038 | 0.606 | erythritol |
| bio_161 | −0.739 | 0 | −0.352 | −0.019 | 0.793 | 0.025 | 0.733 | arabitol |
| bio_499 | −0.733 | 0 | −0.383 | −0.035 | 0.638 | −0.030 | 0.685 | urea |
| bio_714 | −0.732 | 0 | −0.276 | −0.051 | 0.484 | 0.134 | 0.066 | X-16394 |
| bio_525 | −0.730 | 0 | −0.260 | 0.038 | 0.608 | 0.037 | 0.616 | X-11423 |
| bio_242 | −0.718 | 0 | −0.281 | 0.040 | 0.583 | 0.007 | 0.924 | erythronate* |
| bio_214 | −0.710 | 0 | −0.107 | −0.095 | 0.195 | 0.243 | 0.001 | creatinine |
| bio_359 | −0.703 | 0 | −0.245 | 0.029 | 0.697 | 0.007 | 0.923 | myo-inositol |
| bio_385 | −0.699 | 0 | −0.247 | −0.005 | 0.945 | 0.090 | 0.221 | N6-carbamoylthreonyladenosine |
| bio_618 | −0.683 | 0 | −0.168 | 0.000 | 0.996 | 0.000 | 0.996 | X-12749 |
| bio_576 | −0.683 | 0 | −0.425 | −0.016 | 0.833 | −0.039 | 0.593 | X-12104 |
| bio_366 | −0.682 | 0 | −0.415 | −0.029 | 0.690 | 0.118 | 0.106 | N-acetylalanine |
| bio_382 | −0.678 | 0 | −0.324 | −0.055 | 0.458 | 0.043 | 0.560 | N2,N2-dimethylguanosine |
| bio_114 | −0.667 | 0 | −0.144 | −0.007 | 0.921 | 0.033 | 0.652 | 4-acetamidobutanoate |
| bio_566 | −0.658 | 0 | −0.243 | −0.039 | 0.595 | 0.085 | 0.249 | X-11945 |
| bio_26 | −0.644 | 0 | −0.301 | 0.010 | 0.895 | 0.164 | 0.024 | 1-methylhistidine |
| bio_162 | −0.637 | 0 | −0.134 | −0.013 | 0.855 | 0.019 | 0.794 | arabonate |
| bio_375 | −0.635 | 0 | −0.392 | −0.004 | 0.956 | 0.004 | 0.956 | N-formylmethionine |
| bio_69 | −0.633 | 0 | −0.327 | −0.088 | 0.230 | 0.116 | 0.114 | 2-hydroxyisobutyrate |
| bio_510 | −0.614 | 0 | −0.123 | −0.040 | 0.584 | −0.023 | 0.755 | xylonate |
| bio_469 | −0.609 | 0 | −0.317 | −0.080 | 0.273 | −0.001 | 0.986 | succinylcarnitine |
| bio_371 | −0.604 | 0 | −0.193 | −0.053 | 0.472 | 0.058 | 0.427 | N-acetylneuraminate |
| bio_603 | −0.600 | 0 | −0.176 | −0.048 | 0.515 | 0.034 | 0.642 | X-12686 |
| bio_363 | −0.597 | 0 | −0.062 | −0.036 | 0.625 | 0.063 | 0.391 | N-acetyl-1-methylhistidine* |
| bio_298 | −0.593 | 0 | −0.243 | 0.043 | 0.561 | −0.063 | 0.391 | homocitrulline |
| bio_775 | −0.590 | 0 | −0.250 | 0.104 | 0.157 | −0.013 | 0.856 | X-17703 |
| bio_531 | −0.575 | 0 | −0.213 | 0.086 | 0.242 | 0.073 | 0.320 | X-11444 |
| bio_480 | −0.568 | 0 | −0.054 | −0.033 | 0.649 | −0.006 | 0.932 | threitol |
| bio_797 | −0.566 | 0 | −0.389 | 0.025 | 0.734 | −0.155 | 0.033 | X-18887 |
| bio_632 | −0.565 | 0 | −0.258 | 0.172 | 0.018 | 0.073 | 0.318 | X-12846 |
| bio_399 | −0.563 | 0 | −0.268 | 0.211 | 0.004 | −0.115 | 0.115 | p-cresol sulfate |
| bio_110 | −0.557 | 0 | −0.185 | 0.070 | 0.339 | −0.124 | 0.089 | 3-methylglutarylcarnitine (C6) |
| bio_379 | −0.557 | 0 | −0.274 | −0.034 | 0.648 | −0.108 | 0.138 | N1-Methyl-2-pyridone-5- |
| | | | | | | | | carboxamide |
| bio_271 | −0.552 | 0 | −0.180 | −0.066 | 0.368 | 0.096 | 0.191 | glutarylcarnitine (C5) |
| bio_729 | −0.550 | 0 | −0.207 | −0.012 | 0.866 | 0.135 | 0.064 | X-16982 |
| bio_319 | −0.550 | 0 | −0.276 | 0.072 | 0.329 | −0.055 | 0.454 | isobutyrylcarnitine |
| bio_104 | −0.549 | 0 | −0.151 | 0.069 | 0.346 | −0.089 | 0.224 | 3-indoxyl sulfate |
| bio_755 | −0.545 | 0 | −0.110 | 0.115 | 0.115 | −0.016 | 0.823 | X-17357 |
| bio_251 | −0.543 | 2.22E−16 | −0.203 | 0.008 | 0.911 | 0.023 | 0.751 | galactitol (dulcitol) |
| bio_625 | −0.543 | 2.22E−16 | −0.063 | −0.009 | 0.901 | 0.007 | 0.919 | X-12822 |
| bio_651 | −0.539 | 2.22E−16 | −0.131 | −0.086 | 0.242 | 0.020 | 0.781 | X-13837 |
| bio_514 | −0.529 | 1.11E−15 | −0.263 | −0.107 | 0.144 | −0.081 | 0.270 | X-02249 |
| bio_596 | −0.528 | 1.33E−15 | −0.115 | 0.045 | 0.541 | −0.022 | 0.760 | X-12411 |
| bio_652 | −0.528 | 1.33E−15 | −0.121 | −0.052 | 0.483 | 0.027 | 0.716 | X-13844 |
| bio_326 | −0.527 | 1.55E−15 | −0.347 | −0.017 | 0.817 | −0.046 | 0.534 | kynurenine |
| bio_567 | −0.523 | 2.89E−15 | −0.006 | −0.079 | 0.280 | 0.013 | 0.858 | X-12007 |
| bio_643 | −0.520 | 4.66E−15 | −0.114 | −0.064 | 0.381 | 0.131 | 0.072 | X-13553 |
| bio_580 | −0.517 | 6.88E−15 | −0.004 | 0.009 | 0.902 | 0.020 | 0.786 | X-12125 |
| bio_383 | −0.516 | 7.77E−15 | −0.093 | −0.061 | 0.403 | 0.112 | 0.126 | N2,N5-diacetylornithine |
| bio_390 | −0.516 | 7.99E−15 | −0.123 | 0.039 | 0.596 | −0.118 | 0.108 | O-methylcatechol sulfate |
| bio_650 | −0.509 | 2.35E−14 | 0.017 | −0.175 | 0.016 | 0.120 | 0.102 | X-13835 |
| bio_609 | −0.504 | 4.62E−14 | 0.036 | −0.193 | 0.008 | 0.144 | 0.049 | X-12729 |
| bio_621 | −0.500 | 7.88E−14 | 0.021 | −0.036 | 0.624 | 0.033 | 0.656 | X-12814 |
| bio_699 | −0.483 | 7.72E−13 | −0.210 | −0.060 | 0.416 | −0.058 | 0.433 | X-16087 |
| bio_637 | −0.475 | 2.23E−12 | −0.120 | 0.004 | 0.952 | −0.105 | 0.151 | X-12906 |
| bio_629 | −0.474 | 2.31E−12 | 0.003 | −0.055 | 0.458 | 0.019 | 0.800 | X-12831 |
| bio_372 | −0.472 | 2.95E−12 | −0.094 | 0.002 | 0.980 | 0.043 | 0.555 | N-acetylphenylalanine |
| bio_664 | −0.472 | 3.20E−12 | 0.078 | −0.135 | 0.065 | 0.166 | 0.023 | X-14411 |
| bio_411 | −0.470 | 4.05E−12 | −0.165 | 0.190 | 0.009 | −0.079 | 0.283 | phenylacetylglutamine |
| bio_315 | −0.469 | 4.65E−12 | −0.090 | −0.019 | 0.798 | 0.132 | 0.072 | indolelactate |
| bio_117 | −0.468 | 5.33E−12 | −0.150 | 0.035 | 0.638 | 0.096 | 0.190 | 4-acetylphenol sulfate |
| bio_430 | −0.467 | 5.65E−12 | −0.159 | 0.013 | 0.858 | −0.036 | 0.628 | pro-hydroxy-pro |
| bio_78 | −0.467 | 5.97E−12 | −0.098 | −0.141 | 0.054 | 0.202 | 0.005 | 2-methylbutyrylcarnitine (C5) |
| bio_690 | −0.464 | 7.87E−12 | −0.179 | −0.012 | 0.871 | 0.010 | 0.894 | X-15667 |
| bio_208 | −0.458 | 1.67E−11 | −0.122 | 0.019 | 0.797 | 0.060 | 0.411 | citrulline |
| bio_631 | −0.458 | 1.74E−11 | −0.119 | 0.067 | 0.363 | −0.006 | 0.932 | X-12844 |
| bio_324 | −0.458 | 1.77E−11 | −0.124 | 0.085 | 0.245 | 0.015 | 0.842 | isovalerylglycine |
| bio_143 | −0.451 | 4.03E−11 | −0.331 | 0.120 | 0.101 | −0.040 | 0.586 | acetylcarnitine |
| bio_585 | −0.450 | 4.36E−11 | −0.229 | 0.078 | 0.285 | −0.144 | 0.048 | X-12216 |
| bio_522 | −0.449 | 4.65E−11 | −0.135 | −0.022 | 0.768 | 0.107 | 0.142 | X-11334 |
| bio_325 | −0.448 | 5.72E−11 | −0.055 | −0.174 | 0.017 | 0.082 | 0.261 | kynurenate |
| bio_364 | −0.447 | 5.99E−11 | 0.106 | −0.059 | 0.421 | 0.092 | 0.208 | N-acetyl-3-methylhistidine* |
| bio_607 | −0.446 | 6.84E−11 | −0.127 | 0.121 | 0.099 | −0.164 | 0.025 | X-12718 |
| bio_7 | −0.445 | 7.95E−11 | −0.050 | 0.067 | 0.360 | 0.031 | 0.670 | 1,6-anhydroglucose |
| bio_418 | −0.439 | 1.42E−10 | −0.229 | 0.005 | 0.948 | −0.030 | 0.682 | phenylcarnitine* |
| bio_677 | −0.439 | 1.43E−10 | −0.092 | −0.078 | 0.290 | −0.061 | 0.407 | X-15486 |
| bio_599 | −0.438 | 1.66E−10 | −0.186 | −0.080 | 0.273 | 0.136 | 0.063 | X-12511 |
| bio_313 | −0.435 | 2.23E−10 | −0.059 | 0.061 | 0.405 | 0.035 | 0.630 | indoleacetylglutamine |
| bio_678 | −0.434 | 2.67E−10 | −0.204 | 0.051 | 0.486 | −0.022 | 0.764 | X-15503 |
| bio_813 | −0.432 | 3.28E−10 | −0.259 | −0.042 | 0.564 | −0.092 | 0.209 | X-19144 |
| bio_253 | −0.427 | 5.58E−10 | −0.141 | 0.067 | 0.360 | −0.064 | 0.385 | gamma-CEHC glucuronide* |
| bio_627 | −0.424 | 7.30E−10 | −0.101 | 0.050 | 0.494 | 0.146 | 0.045 | X-12828 |
| bio_470 | −0.421 | 9.91E−10 | −0.080 | 0.090 | 0.218 | 0.019 | 0.797 | sucrose |
| bio_575 | −0.420 | 1.13E−09 | −0.125 | 0.072 | 0.326 | −0.017 | 0.813 | X-12100 |
| bio_721 | −0.416 | 1.64E−09 | −0.126 | 0.031 | 0.670 | 0.081 | 0.271 | X-16674 |
| bio_821 | −0.416 | 1.77E−09 | −0.325 | −0.037 | 0.614 | −0.029 | 0.689 | X-19437 |
| bio_640 | −0.410 | 3.09E−09 | −0.293 | 0.066 | 0.369 | 0.092 | 0.207 | X-13435 |
| bio_266 | −0.409 | 3.56E−09 | −0.100 | 0.032 | 0.661 | −0.082 | 0.266 | glucuronate |
| bio_438 | −0.408 | 3.66E−09 | −0.252 | −0.063 | 0.393 | 0.398 | 0.000 | pyroglutamine* |
| bio_409 | −0.406 | 4.64E−09 | −0.163 | −0.062 | 0.400 | 0.088 | 0.231 | phenol sulfate |
| bio_739 | −0.405 | 5.02E−09 | −0.119 | 0.079 | 0.284 | 0.098 | 0.182 | X-17178 |
| bio_498 | −0.401 | 7.78E−09 | −0.203 | −0.197 | 0.007 | 0.222 | 0.002 | urate |
| bio_368 | −0.399 | 8.82E−09 | 0.056 | −0.089 | 0.222 | 0.442 | 0.000 | N-acetylcarnosine |
| bio_127 | −0.397 | 1.07E−08 | −0.200 | −0.134 | 0.068 | 0.021 | 0.775 | 5-acetylamino-6-amino-3- |
| | | | | | | | | methyluracil |
| bio_197 | −0.396 | 1.23E−08 | −0.161 | −0.029 | 0.696 | −0.097 | 0.186 | catechol sulfate |
| bio_228 | −0.392 | 1.79E−08 | −0.162 | 0.036 | 0.623 | 0.043 | 0.559 | dimethylglycine |
| bio_776 | −0.391 | 1.89E−08 | −0.147 | 0.053 | 0.467 | 0.033 | 0.654 | X-17706 |
| bio_818 | −0.386 | 2.97E−08 | −0.322 | 0.110 | 0.132 | −0.216 | 0.003 | X-19429 |
| bio_595 | −0.386 | 3.15E−08 | −0.133 | 0.012 | 0.868 | −0.075 | 0.304 | X-12410 |
| bio_367 | −0.382 | 4.56E−08 | −0.077 | 0.072 | 0.328 | 0.091 | 0.215 | N-acetylaspartate (NAA) |
| bio_123 | −0.380 | 5.20E−08 | −0.113 | 0.017 | 0.814 | 0.093 | 0.204 | 4-hydroxyphenylacetate |
| bio_605 | −0.378 | 6.18E−08 | −0.185 | 0.138 | 0.059 | −0.149 | 0.041 | X-12705 |
| bio_111 | −0.378 | 6.54E−08 | −0.086 | −0.076 | 0.297 | 0.061 | 0.409 | 3-methylhistidine |
| bio_604 | −0.377 | 7.15E−08 | 0.055 | −0.082 | 0.266 | 0.014 | 0.844 | X-12704 |
| bio_534 | −0.376 | 7.25E−08 | −0.164 | 0.101 | 0.169 | 0.027 | 0.714 | X-11470 |
| bio_518 | −0.373 | 9.46E−08 | −0.186 | −0.046 | 0.529 | −0.073 | 0.318 | X-11261 |
| bio_573 | −0.371 | 1.21E−07 | −0.084 | 0.003 | 0.962 | 0.020 | 0.787 | X-12092 |
| bio_150 | −0.367 | 1.71E−07 | −0.128 | −0.020 | 0.783 | 0.190 | 0.009 | allantoin |
| bio_591 | −0.364 | 2.08E−07 | 0.052 | −0.025 | 0.729 | 0.089 | 0.223 | X-12263 |
| bio_454 | −0.362 | 2.44E−07 | −0.145 | −0.014 | 0.851 | −0.050 | 0.410 | scyllo-inositol |
| bio_405 | −0.361 | 2.63E−07 | −0.137 | 0.123 | 0.093 | −0.228 | 0.002 | pantothenate |
| bio_248 | −0.361 | 2.83E−07 | −0.177 | 0.027 | 0.711 | 0.074 | 0.314 | fucose |
| bio_548 | −0.355 | 4.36E−07 | −0.072 | −0.033 | 0.657 | −0.049 | 0.509 | X-11640 |
| bio_594 | −0.349 | 7.00E−07 | −0.077 | 0.024 | 0.740 | −0.056 | 0.442 | X-12407 |
| bio_753 | −0.345 | 1.01E−06 | −0.194 | 0.071 | 0.331 | −0.064 | 0.383 | X-17354 |
| bio_160 | −0.343 | 1.18E−06 | −0.097 | −0.149 | 0.042 | 0.090 | 0.222 | arabinose |
| bio_624 | −0.343 | 1.20E−06 | 0.128 | 0.055 | 0.456 | 0.006 | 0.930 | X-12820 |
| bio_490 | −0.342 | 1.28E−06 | −0.016 | −0.068 | 0.357 | −0.023 | 0.752 | trimethylamine N-oxide |
| bio_369 | −0.341 | 1.35E−06 | −0.151 | −0.066 | 0.370 | −0.010 | 0.888 | N-acetylglycine |
| bio_120 | −0.337 | 1.80E−06 | −0.014 | 0.006 | 0.940 | −0.050 | 0.498 | 4-guanidinobutanoate |
| bio_122 | −0.335 | 2.13E−06 | −0.172 | 0.043 | 0.560 | −0.122 | 0.096 | 4-hydroxyhippurate |
| bio_444 | −0.334 | 2.30E−06 | −0.110 | 0.033 | 0.650 | −0.114 | 0.120 | quinolinate |
| bio_661 | −0.332 | 2.79E−06 | 0.107 | 0.048 | 0.514 | −0.072 | 0.329 | X-14352 |
| bio_608 | −0.331 | 2.96E−06 | −0.105 | 0.035 | 0.636 | −0.036 | 0.622 | X-12719 |
| bio_623 | −0.330 | 3.01E−06 | −0.034 | −0.087 | 0.237 | 0.129 | 0.079 | X-12818 |
| bio_786 | −0.330 | 3.02E−06 | 0.137 | 0.027 | 0.710 | −0.007 | 0.924 | X-18345 |
| bio_103 | −0.330 | 3.04E−06 | −0.102 | 0.004 | 0.960 | 0.101 | 0.167 | 3-hydroxysebacate |
| bio_106 | −0.330 | 3.14E−06 | −0.129 | −0.039 | 0.596 | 0.145 | 0.048 | 3-methyl catechol sulfate 1 |
| bio_205 | −0.329 | 3.33E−06 | −0.260 | 0.041 | 0.575 | −0.007 | 0.929 | cis-4-decenoyl carnitine |
| bio_613 | −0.323 | 5.13E−06 | −0.128 | 0.021 | 0.780 | −0.004 | 0.952 | X-12739 |
| bio_357 | −0.322 | 5.74E−06 | −0.165 | 0.058 | 0.430 | 0.118 | 0.107 | metoprolol acid metabolite* |
| bio_442 | −0.320 | 6.53E−06 | −0.169 | −0.010 | 0.893 | 0.046 | 0.535 | quinate |
| bio_628 | −0.320 | 6.68E−06 | −0.295 | 0.044 | 0.551 | −0.089 | 0.225 | X-12830 |
| bio_648 | −0.317 | 8.19E−06 | −0.039 | 0.052 | 0.483 | −0.090 | 0.218 | X-13726 |
| bio_87 | −0.316 | 8.87E−06 | −0.175 | −0.149 | 0.041 | 0.254 | 0.000 | 21-hydroxypregnenolone |
| | | | | | | | | disulfate |
| bio_125 | −0.312 | 0.0000116 | 0.000 | 0.049 | 0.507 | −0.052 | 0.476 | 4-methylcatechol sulfate |
| bio_767 | −0.311 | 0.0000125 | −0.148 | 0.054 | 0.463 | 0.108 | 0.141 | X-17612 |
| bio_204 | −0.310 | 0.0000129 | −0.227 | 0.029 | 0.689 | −0.129 | 0.077 | cinnamoylglycine |
| bio_606 | −0.306 | 0.0000175 | 0.156 | −0.033 | 0.658 | 0.051 | 0.486 | X-12712 |
| bio_558 | −0.303 | 0.0000203 | −0.197 | 0.114 | 0.120 | 0.020 | 0.785 | X-11840 |
| bio_579 | −0.302 | 0.0000228 | −0.122 | −0.016 | 0.830 | 0.029 | 0.696 | X-12116 |
| bio_215 | −0.297 | 0.0000309 | −0.179 | 0.020 | 0.787 | 0.027 | 0.714 | cyclo(gly-pro) |
| bio_486 | −0.296 | 0.0000324 | 0.066 | −0.152 | 0.038 | −0.004 | 0.957 | tiglyl carnitine |
| bio_757 | −0.294 | 0.0000383 | −0.231 | 0.030 | 0.686 | −0.034 | 0.640 | X-17369 |
| bio_293 | −0.293 | 0.0000407 | −0.119 | 0.017 | 0.818 | 0.020 | 0.785 | hippurate |
| bio_561 | −0.293 | 0.0000412 | −0.016 | 0.079 | 0.281 | 0.075 | 0.305 | X-11850 |
| bio_590 | −0.291 | 0.0000459 | 0.020 | 0.030 | 0.686 | 0.071 | 0.334 | X-12261 |
| bio_380 | −0.291 | 0.0000468 | −0.144 | 0.032 | 0.666 | −0.126 | 0.086 | N1-methyladenosine |
| bio_611 | −0.286 | 0.0000629 | −0.021 | −0.008 | 0.917 | 0.157 | 0.032 | X-12731 |
| bio_145 | −0.286 | 0.0000635 | −0.270 | 0.046 | 0.532 | −0.009 | 0.898 | acisoga |
| bio_349 | −0.285 | 0.0000691 | −0.056 | 0.032 | 0.665 | −0.057 | 0.434 | mannitol |
| bio_285 | −0.281 | 0.0000838 | −0.109 | 0.144 | 0.048 | 0.082 | 0.266 | glycylglycine |
| bio_227 | −0.280 | 0.0000934 | −0.254 | 0.026 | 0.726 | −0.039 | 0.593 | dimethylarginine (SDMA +ADMA) |
| bio_440 | −0.279 | 0.0000951 | 0.119 | −0.019 | 0.798 | 0.012 | 0.870 | pyrophosphate (PPI) |
| bio_447 | −0.277 | 0.000107 | −0.274 | 0.055 | 0.457 | −0.137 | 0.060 | ribose |
| bio_396 | −0.273 | 0.0001427 | −0.008 | 0.012 | 0.866 | 0.062 | 0.400 | ornithine |
| bio_626 | −0.272 | 0.0001508 | −0.071 | −0.088 | 0.232 | 0.061 | 0.409 | X-12824 |
| bio_250 | −0.271 | 0.0001535 | −0.217 | −0.024 | 0.744 | 0.049 | 0.505 | furosemide |
| bio_550 | −0.271 | 0.0001582 | −0.091 | −0.032 | 0.659 | 0.151 | 0.038 | X-11787 |
| bio_93 | −0.269 | 0.0001713 | −0.217 | −0.110 | 0.132 | −0.056 | 0.442 | 3-dehydrocarnitine* |
| bio_586 | −0.267 | 0.0001954 | 0.025 | −0.136 | 0.062 | 0.071 | 0.334 | X-12221 |
| bio_570 | −0.267 | 0.0002028 | −0.088 | −0.096 | 0.192 | 0.052 | 0.481 | X-12039 |
| bio_630 | −0.266 | 0.0002072 | −0.048 | −0.037 | 0.613 | −0.061 | 0.407 | X-12832 |
| bio_719 | −0.264 | 0.0002329 | 0.030 | 0.020 | 0.784 | 0.077 | 0.295 | X-16617 |
| bio_370 | −0.264 | 0.0002396 | −0.150 | 0.048 | 0.516 | −0.015 | 0.842 | N-acetylmethionine |
| bio_91 | −0.262 | 0.0002605 | −0.048 | 0.060 | 0.417 | 0.086 | 0.239 | 3-aminoisobutyrate |
| bio_772 | −0.262 | 0.0002605 | −0.194 | −0.079 | 0.284 | −0.011 | 0.885 | X-17686 |
| bio_63 | −0.261 | 0.0002862 | −0.130 | 0.061 | 0.406 | −0.076 | 0.300 | 2-hydroxyacetominophen |
| | | | | | | | | sulfate* |
| bio_808 | −0.260 | 0.0002871 | −0.026 | 0.057 | 0.437 | −0.017 | 0.812 | X-19132 |
| bio_481 | −0.260 | 0.000289 | −0.138 | 0.020 | 0.788 | −0.181 | 0.013 | threonate |
| bio_432 | −0.260 | 0.0003005 | −0.127 | 0.097 | 0.187 | 0.145 | 0.047 | propionylcarnitine |
| bio_616 | −0.258 | 0.0003318 | −0.144 | 0.025 | 0.732 | 0.013 | 0.860 | X-12742 |
| bio_185 | −0.256 | 0.0003737 | −0.206 | 0.031 | 0.669 | 0.068 | 0.354 | butyrylcarnitine |
| bio_538 | −0.254 | 0.0004015 | −0.136 | −0.031 | 0.677 | 0.026 | 0.727 | X-11521 |
| bio_384 | −0.253 | 0.0004362 | −0.169 | −0.056 | 0.446 | −0.058 | 0.428 | N6-acetyllysine |
| bio_647 | −0.252 | 0.0004528 | −0.124 | 0.066 | 0.372 | 0.114 | 0.118 | X-13699 |
| bio_94 | −0.252 | 0.0004587 | −0.151 | −0.022 | 0.767 | 0.138 | 0.060 | 3-ethylphenylsulfate* |
| bio_97 | −0.249 | 0.0005306 | −0.165 | −0.004 | 0.961 | 0.076 | 0.303 | 3-hydroxycotinine glucuronide |
| bio_461 | −0.248 | 0.0005712 | −0.178 | −0.074 | 0.311 | −0.083 | 0.257 | stachydrine |
| bio_4 | −0.248 | 0.000583 | −0.016 | −0.048 | 0.515 | 0.004 | 0.954 | 1,3-dimethylurate |
| bio_453 | −0.245 | 0.0006533 | −0.004 | −0.119 | 0.103 | 0.073 | 0.321 | sarcosine (N-Methylglycine) |
| bio_107 | −0.244 | 0.0007069 | −0.104 | −0.048 | 0.514 | 0.192 | 0.008 | 3-methyl catechol sulfate 2 |
| bio_126 | −0.244 | 0.000715 | −0.187 | −0.066 | 0.365 | 0.045 | 0.541 | 4-vinylphenol sulfate |
| bio_732 | −0.244 | 0.0007182 | −0.059 | −0.083 | 0.260 | −0.053 | 0.472 | X-17138 |
| bio_577 | −0.243 | 0.0007318 | −0.035 | −0.061 | 0.402 | −0.109 | 0.138 | X-12107 |
| bio_61 | −0.243 | 0.0007538 | −0.116 | −0.085 | 0.247 | 0.137 | 0.060 | 2-ethylphenylsulfate |
| bio_92 | −0.242 | 0.0008001 | −0.062 | 0.014 | 0.848 | −0.098 | 0.179 | 3-carboxy-4-methyl-5-propyl-2- |
| | | | | | | | | furanpropanoate (CMPF) |
| bio_427 | −0.241 | 0.0008358 | −0.116 | −0.065 | 0.376 | −0.062 | 0.400 | pregnanediol-3-glucuronide |
| bio_202 | −0.238 | 0.0009511 | −0.150 | 0.072 | 0.328 | 0.091 | 0.217 | choline |
| bio_398 | −0.237 | 0.0010216 | −0.111 | 0.093 | 0.205 | −0.083 | 0.261 | p-acetamidophenylglucuronide |
| bio_823 | −0.235 | 0.0011091 | −0.023 | 0.018 | 0.805 | 0.084 | 0.254 | X-19441 |
| bio_701 | −0.231 | 0.001376 | −0.054 | 0.054 | 0.460 | −0.003 | 0.968 | X-16123 |
| bio_748 | −0.230 | 0.0014662 | −0.129 | 0.031 | 0.675 | 0.016 | 0.832 | X-17327 |
| bio_512 | −0.227 | 0.0016756 | 0.019 | 0.048 | 0.513 | −0.022 | 0.763 | xylulose |
| bio_740 | −0.227 | 0.0016813 | −0.136 | 0.075 | 0.308 | −0.003 | 0.964 | X-17179 |
| bio_806 | −0.226 | 0.0017252 | −0.038 | 0.008 | 0.914 | −0.030 | 0.686 | X-18965 |
| bio_752 | −0.225 | 0.0018597 | −0.113 | −0.162 | 0.026 | −0.203 | 0.005 | X-17353 |
| bio_692 | −0.224 | 0.0019288 | 0.039 | 0.126 | 0.085 | 0.029 | 0.697 | X-15708 |
| bio_758 | −0.222 | 0.002131 | −0.194 | 0.108 | 0.139 | −0.023 | 0.753 | X-17371 |
| bio_348 | −0.222 | 0.0021801 | 0.036 | 0.086 | 0.242 | −0.026 | 0.724 | maltose |
| bio_252 | −0.220 | 0.0023795 | −0.097 | 0.046 | 0.531 | −0.043 | 0.556 | gamma-CEHC |
| bio_389 | −0.219 | 0.0024257 | −0.098 | −0.003 | 0.963 | 0.140 | 0.055 | o-cresol sulfate |
| bio_645 | −0.217 | 0.0026823 | −0.126 | 0.154 | 0.035 | −0.079 | 0.280 | X-13689 |
| bio_687 | −0.217 | 0.0027146 | −0.030 | −0.194 | 0.008 | 0.095 | 0.193 | X-15646 |
| bio_820 | −0.216 | 0.0028754 | −0.068 | 0.106 | 0.150 | 0.102 | 0.165 | X-19434 |
| bio_52 | −0.213 | 0.00332 | −0.023 | −0.062 | 0.401 | 0.091 | 0.216 | 2,3-dihydroxyisovalerate |
| bio_737 | −0.212 | 0.0034788 | −0.162 | 0.124 | 0.089 | −0.110 | 0.133 | X-17175 |
| bio_578 | −0.211 | 0.0035097 | −0.017 | −0.052 | 0.476 | 0.058 | 0.428 | X-12108 |
| bio_693 | −0.207 | 0.0042502 | 0.002 | −0.232 | 0.001 | 0.045 | 0.542 | X-15728 |
| bio_679 | −0.207 | 0.0043423 | −0.100 | 0.015 | 0.834 | −0.062 | 0.397 | X-15523 |
| bio_622 | −0.206 | 0.0043867 | −0.128 | −0.065 | 0.377 | 0.004 | 0.954 | X-12816 |
| bio_287 | −0.206 | 0.0044819 | −0.180 | −0.008 | 0.911 | −0.130 | 0.075 | glycylvaline |
| bio_771 | −0.206 | 0.0045689 | −0.029 | −0.015 | 0.837 | −0.010 | 0.896 | X-17685 |
| bio_134 | −0.204 | 0.0048776 | −0.055 | −0.114 | 0.119 | 0.215 | 0.003 | 5alpha-androstan- |
| | | | | | | | | 3beta,17alpha-diol disulfate |
| bio_142 | −0.200 | 0.0059244 | −0.053 | −0.152 | 0.038 | 0.135 | 0.065 | 9-methyluric acid |
| bio_152 | −0.199 | 0.0060547 | −0.088 | 0.059 | 0.421 | −0.120 | 0.100 | alpha-CEHC glucuronide* |
| bio_356 | −0.199 | 0.0062075 | −0.148 | 0.026 | 0.722 | 0.137 | 0.061 | metoprolol |
| bio_587 | −0.198 | 0.0063365 | −0.079 | −0.118 | 0.107 | 0.049 | 0.504 | X-12230 |
| bio_381 | −0.198 | 0.0063399 | −0.081 | −0.026 | 0.722 | 0.081 | 0.270 | N1-methylguanosine |
| bio_277 | −0.198 | 0.0063638 | −0.266 | 0.093 | 0.204 | −0.230 | 0.001 | glycine |
| bio_450 | −0.198 | 0.0064175 | −0.035 | −0.111 | 0.130 | −0.166 | 0.022 | saccharin |
| bio_247 | −0.198 | 0.0064387 | −0.005 | −0.009 | 0.907 | −0.028 | 0.705 | fructose |
| bio_822 | −0.197 | 0.0066519 | −0.016 | 0.044 | 0.550 | 0.064 | 0.381 | X-19440 |
| bio_249 | −0.195 | 0.0073692 | 0.005 | 0.131 | 0.073 | 0.089 | 0.225 | fumarate |
| bio_810 | −0.194 | 0.0075849 | −0.060 | −0.073 | 0.319 | 0.028 | 0.708 | X-19136 |
| bio_686 | −0.193 | 0.0079155 | −0.052 | −0.005 | 0.943 | 0.131 | 0.072 | X-15636 |
| bio_593 | −0.193 | 0.0079498 | −0.027 | −0.161 | 0.028 | 0.026 | 0.727 | X-12329 |
| bio_487 | −0.192 | 0.0082151 | −0.078 | −0.010 | 0.892 | 0.119 | 0.103 | trans-4-hydroxyproline |
| bio_765 | −0.191 | 0.0085916 | −0.015 | −0.019 | 0.794 | 0.110 | 0.132 | X-17459 |
| bio_99 | −0.190 | 0.008726 | −0.028 | 0.072 | 0.329 | −0.033 | 0.656 | 3-hydroxyhippurate |
| bio_292 | −0.189 | 0.0091413 | −0.216 | 0.067 | 0.362 | −0.006 | 0.930 | hexanoylcarnitine |
| bio_597 | −0.189 | 0.0094109 | −0.098 | 0.063 | 0.390 | −0.115 | 0.117 | X-12435 |
| bio_429 | −0.189 | 0.0094595 | −0.127 | −0.078 | 0.287 | 0.230 | 0.001 | pregnenolone sulfate |
| bio_654 | −0.188 | 0.0094618 | 0.026 | 0.103 | 0.159 | −0.035 | 0.635 | X-13866 |
| bio_188 | −0.188 | 0.0095537 | −0.182 | 0.048 | 0.510 | −0.111 | 0.129 | campesterol |
| bio_207 | −0.184 | 0.0113099 | −0.240 | 0.290 | 0.000 | −0.137 | 0.061 | citrate |
| bio_199 | −0.183 | 0.0115948 | −0.041 | −0.050 | 0.500 | −0.030 | 0.687 | chiro-inositol |
| bio_511 | −0.183 | 0.0118181 | 0.047 | 0.018 | 0.804 | −0.119 | 0.103 | xylose |
| bio_90 | −0.183 | 0.012006 | −0.107 | 0.032 | 0.666 | −0.142 | 0.051 | 3-(N-acetyl-L-cystein-S-yl) |
| | | | | | | | | acetaminophen* |
| bio_36 | −0.181 | 0.0130207 | −0.109 | −0.006 | 0.940 | −0.061 | 0.402 | 1- |
| | | | | | | | | palmitoylglycerophosphoethanol- |
| | | | | | | | | amine |
| bio_105 | −0.180 | 0.0132408 | −0.238 | 0.025 | 0.732 | −0.050 | 0.494 | 3-methoxytyrosine |
| bio_392 | −0.180 | 0.013368 | −0.187 | 0.057 | 0.438 | 0.011 | 0.884 | octanoylcarnitine |
| bio_720 | −0.176 | 0.0155587 | −0.106 | 0.032 | 0.665 | −0.086 | 0.241 | X-16649 |
| bio_116 | −0.175 | 0.0161019 | −0.089 | −0.082 | 0.266 | −0.070 | 0.340 | 4-acetaminophen sulfate |
| bio_311 | −0.174 | 0.0167752 | −0.028 | −0.027 | 0.708 | 0.150 | 0.040 | imidazole propionate |
| bio_742 | −0.173 | 0.0171658 | −0.088 | −0.174 | 0.017 | 0.135 | 0.064 | X-17185 |
| bio_403 | −0.173 | 0.0171759 | −0.113 | 0.102 | 0.164 | −0.129 | 0.077 | palmitoyl sphingomyelin |
| bio_710 | −0.173 | 0.017477 | −0.041 | 0.007 | 0.925 | 0.001 | 0.991 | X-16136 |
| bio_824 | −0.173 | 0.0176776 | −0.147 | 0.126 | 0.084 | −0.019 | 0.801 | X-19451 |
| bio_671 | −0.172 | 0.01813 | −0.227 | 0.056 | 0.445 | −0.008 | 0.911 | X-14947 |
| bio_828 | −0.171 | 0.0186794 | −0.184 | 0.020 | 0.787 | −0.121 | 0.098 | X-19616 |
| bio_557 | −0.171 | 0.0190951 | −0.180 | −0.010 | 0.888 | −0.158 | 0.031 | X-11838 |
| bio_422 | −0.170 | 0.0194338 | −0.004 | 0.053 | 0.471 | −0.191 | 0.008 | phosphate |
| bio_217 | −0.169 | 0.0202035 | −0.198 | 0.066 | 0.368 | 0.010 | 0.890 | decanoylcarnitine |
| bio_31 | −0.168 | 0.0206832 | −0.109 | −0.052 | 0.477 | 0.034 | 0.645 | 1- |
| | | | | | | | | oleoylglycerophosphoethanolamine |
| bio_812 | −0.168 | 0.0212182 | −0.090 | −0.032 | 0.664 | −0.118 | 0.107 | X-19140 |
| bio_220 | −0.168 | 0.0213423 | −0.072 | 0.057 | 0.439 | 0.372 | 0.000 | deoxycarnitine |
| bio_819 | −0.167 | 0.0216181 | −0.016 | 0.046 | 0.529 | 0.064 | 0.383 | X-19430 |
| bio_809 | −0.166 | 0.0226622 | −0.095 | −0.088 | 0.230 | −0.054 | 0.464 | X-19134 |
| bio_365 | −0.164 | 0.0240226 | 0.180 | −0.044 | 0.552 | 0.117 | 0.111 | N-acetyl-beta-alanine |
| bio_144 | −0.163 | 0.0252372 | −0.077 | 0.008 | 0.911 | −0.035 | 0.636 | acetylphosphate |
| bio_783 | −0.163 | 0.0254371 | 0.154 | 0.070 | 0.343 | 0.048 | 0.514 | X-18273 |
| bio_556 | −0.162 | 0.0266866 | −0.205 | 0.047 | 0.526 | −0.071 | 0.336 | X-11835 |
| bio_768 | −0.161 | 0.0268359 | −0.057 | 0.024 | 0.745 | 0.030 | 0.686 | X-17626 |
| bio_782 | −0.161 | 0.0272814 | 0.153 | 0.070 | 0.338 | 0.050 | 0.500 | X-18271 |
| bio_698 | −0.161 | 0.0275025 | 0.155 | 0.072 | 0.329 | 0.050 | 0.495 | X-16083 |
| bio_386 | −0.161 | 0.0276486 | 0.201 | −0.094 | 0.202 | 0.057 | 0.437 | naproxen |
| bio_452 | −0.160 | 0.0277025 | −0.141 | 0.051 | 0.489 | −0.078 | 0.287 | salicyluric glucuronide* |
| bio_695 | −0.160 | 0.0277418 | 0.154 | 0.069 | 0.350 | 0.049 | 0.507 | X-15737 |
| bio_3 | −0.160 | 0.0283451 | 0.023 | −0.151 | 0.039 | −0.025 | 0.730 | 1,3,7-trimethylurate |
| bio_167 | −0.159 | 0.0291914 | −0.249 | 0.063 | 0.393 | −0.160 | 0.028 | aspartate |
| bio_602 | −0.159 | 0.0296035 | 0.153 | 0.070 | 0.338 | 0.050 | 0.500 | X-12609 |
| bio_483 | −0.158 | 0.0300564 | −0.009 | 0.050 | 0.493 | 0.049 | 0.508 | threonylphenylalanine |
| bio_244 | −0.158 | 0.03023 | −0.030 | 0.040 | 0.582 | 0.144 | 0.048 | ethanolamine |
| bio_286 | −0.157 | 0.0311647 | −0.109 | −0.017 | 0.816 | 0.038 | 0.603 | glycylphenylalanine |
| bio_426 | −0.157 | 0.0313556 | −0.081 | −0.073 | 0.319 | 0.290 | 0.000 | pregn steroid monosulfate* |
| bio_789 | −0.157 | 0.0314169 | 0.140 | 0.067 | 0.363 | 0.040 | 0.589 | X-18554 |
| bio_119 | −0.156 | 0.032725 | −0.023 | −0.238 | 0.001 | 0.364 | 0.000 | 4-androsten-3beta,17beta-diol |
| | | | | | | | | disulfate 2* |
| bio_589 | −0.154 | 0.0348015 | −0.175 | −0.010 | 0.891 | −0.016 | 0.833 | X-12254 |
| bio_684 | −0.153 | 0.0355631 | −0.033 | −0.096 | 0.189 | 0.036 | 0.628 | X-15606 |
| bio_139 | −0.153 | 0.03565 | −0.108 | −0.019 | 0.795 | 0.107 | 0.145 | 7-dehydrocholesterol |
| bio_516 | −0.153 | 0.0363029 | 0.020 | −0.013 | 0.861 | 0.022 | 0.767 | X-10458 |
| bio_762 | −0.149 | 0.0415836 | −0.110 | −0.007 | 0.928 | 0.060 | 0.413 | X-17444 |
| bio_441 | −0.148 | 0.0421316 | −0.058 | 0.125 | 0.088 | 0.014 | 0.851 | pyruvate |
| bio_471 | −0.148 | 0.042516 | 0.055 | −0.092 | 0.210 | −0.037 | 0.616 | tartarate |
| bio_76 | −0.144 | 0.048514 | −0.154 | 0.069 | 0.346 | −0.100 | 0.174 | 2-methoxyacetaminophen |
| | | | | | | | | glucuronide* |
| bio_774 | −0.142 | 0.0511249 | 0.147 | 0.019 | 0.799 | −0.103 | 0.160 | X-17692 |
| bio_290 | −0.142 | 0.0512259 | −0.045 | −0.111 | 0.129 | 0.044 | 0.553 | heptanoate (7:0) |
| bio_571 | −0.142 | 0.0514547 | −0.213 | 0.111 | 0.130 | −0.086 | 0.243 | X-12056 |
| bio_549 | −0.139 | 0.0566989 | 0.038 | −0.105 | 0.150 | −0.053 | 0.473 | X-11727 |
| bio_790 | −0.138 | 0.0592048 | −0.078 | 0.002 | 0.974 | 0.112 | 0.125 | X-18604 |
| bio_462 | −0.138 | 0.0592785 | −0.022 | 0.036 | 0.623 | 0.010 | 0.890 | stearamide |
| bio_546 | −0.138 | 0.0595991 | −0.060 | 0.089 | 0.223 | 0.110 | 0.132 | X-11612 |
| bio_42 | −0.137 | 0.0606841 | −0.070 | −0.006 | 0.936 | 0.123 | 0.092 | 1- |
| | | | | | | | | stearoylglycerophosphoethanol- |
| | | | | | | | | amine |
| bio_763 | −0.135 | 0.0646611 | 0.125 | 0.033 | 0.649 | 0.011 | 0.878 | X-17447 |
| bio_655 | −0.135 | 0.0650298 | −0.070 | −0.014 | 0.854 | 0.087 | 0.237 | X-13891 |
| bio_526 | −0.135 | 0.0654423 | −0.176 | −0.069 | 0.349 | −0.182 | 0.012 | X-11437 |
| bio_73 | −0.134 | 0.0672856 | 0.048 | −0.024 | 0.748 | 0.097 | 0.187 | 2-linoleoylglycerol (2- |
| | | | | | | | | monolinolein) |
| bio_299 | −0.133 | 0.0695185 | −0.094 | 0.023 | 0.755 | 0.020 | 0.780 | homostachydrine* |
| bio_83 | −0.132 | 0.0698832 | −0.025 | −0.017 | 0.817 | −0.005 | 0.945 | 2-palmitoylglycerophosphoethanolamine* |
| bio_515 | −0.131 | 0.0735047 | −0.155 | −0.040 | 0.582 | −0.166 | 0.023 | X-10346 |
| bio_582 | −0.131 | 0.0739398 | 0.067 | −0.104 | 0.155 | 0.052 | 0.482 | X-12189 |
| bio_70 | −0.126 | 0.0854231 | −0.104 | −0.194 | 0.007 | 0.100 | 0.172 | 2-hydroxyoctanoate |
| bio_177 | −0.125 | 0.0875375 | −0.068 | 0.057 | 0.440 | −0.134 | 0.066 | beta-sitosterol |
| bio_397 | −0.124 | 0.0887611 | −0.064 | 0.081 | 0.268 | 0.136 | 0.063 | oxypurinol |
| bio_517 | −0.124 | 0.0893611 | −0.044 | −0.170 | 0.019 | 0.033 | 0.657 | X-11247 |
| bio_377 | −0.124 | 0.0898242 | −0.100 | 0.121 | 0.097 | 0.013 | 0.863 | N-methyl-acetaminophen sulfate |
| | | | | | | | | 1* |
| bio_88 | −0.124 | 0.0898865 | 0.006 | −0.013 | 0.856 | 0.185 | 0.011 | 3-(4-hydroxyphenyl)lactate |
| bio_588 | −0.124 | 0.0898995 | −0.020 | −0.077 | 0.293 | 0.041 | 0.581 | X-12231 |
| bio_532 | −0.123 | 0.0913477 | −0.030 | −0.106 | 0.150 | 0.037 | 0.617 | X-11452 |
| bio_222 | −0.123 | 0.0919636 | 0.054 | −0.086 | 0.238 | 0.080 | 0.275 | desmethylnaproxen sulfate* |
| bio_22 | −0.122 | 0.094328 | −0.031 | 0.017 | 0.814 | 0.056 | 0.445 | 1-linoleoylglycerol (1- |
| | | | | | | | | monolinolein) |
| bio_583 | −0.120 | 0.1008739 | −0.100 | 0.087 | 0.234 | 0.147 | 0.044 | X-12195 |
| bio_305 | −0.120 | 0.1019326 | −0.072 | 0.014 | 0.845 | 0.066 | 0.367 | hydroxybutyrylcarnitine* |
| bio_756 | −0.120 | 0.1021427 | −0.090 | −0.001 | 0.994 | −0.036 | 0.621 | X-17367 |
| bio_612 | −0.119 | 0.1024499 | 0.045 | −0.103 | 0.160 | 0.015 | 0.840 | X-12734 |
| bio_209 | −0.118 | 0.1078814 | −0.077 | 0.070 | 0.340 | −0.203 | 0.005 | cortisol |
| bio_716 | −0.117 | 0.1086738 | −0.085 | −0.004 | 0.956 | −0.012 | 0.872 | X-16564 |
| bio_175 | −0.117 | 0.1097999 | 0.052 | −0.299 | 0.000 | 0.191 | 0.009 | beta-alanine |
| bio_54 | −0.115 | 0.1160224 | −0.123 | 0.170 | 0.019 | 0.061 | 0.406 | 2-aminoheptanoic acid |
| bio_749 | −0.115 | 0.1175189 | −0.074 | −0.027 | 0.710 | −0.012 | 0.873 | X-17328 |
| bio_269 | −0.113 | 0.123527 | −0.162 | −0.020 | 0.782 | −0.074 | 0.311 | glutamine-leucine |
| bio_601 | −0.112 | 0.124727 | −0.094 | 0.101 | 0.166 | −0.095 | 0.193 | X-12543 |
| bio_666 | −0.112 | 0.1255765 | −0.077 | −0.028 | 0.706 | −0.027 | 0.713 | X-14588 |
| bio_722 | −0.111 | 0.1286481 | −0.133 | 0.093 | 0.202 | −0.008 | 0.913 | X-16932 |
| bio_24 | −0.111 | 0.1290896 | −0.038 | −0.059 | 0.425 | 0.160 | 0.029 | 1- |
| | | | | | | | | linoleoylglycerophosphoethanol- |
| | | | | | | | | amine* |
| bio_312 | −0.109 | 0.1350226 | 0.016 | 0.050 | 0.493 | 0.143 | 0.051 | indoleacetate |
| bio_201 | −0.109 | 0.138408 | −0.022 | 0.083 | 0.260 | −0.081 | 0.271 | cholesterol |
| bio_519 | −0.108 | 0.1388734 | −0.119 | 0.037 | 0.187 | 0.001 | 0.989 | X-11299 |
| bio_77 | −0.108 | 0.1418035 | −0.172 | 0.007 | 0.927 | −0.126 | 0.086 | 2-methoxyacetaminophen |
| | | | | | | | | sulfate* |
| bio_130 | −0.107 | 0.1422117 | −0.055 | −0.188 | 0.010 | −0.062 | 0.398 | 5-hydroxymethyl-2-furoic acid |
| bio_288 | −0.107 | 0.1446012 | −0.159 | 0.035 | 0.637 | −0.047 | 0.520 | guanosine |
| bio_574 | −0.107 | 0.1450244 | 0.035 | −0.018 | 0.811 | 0.009 | 0.905 | X-12093 |
| bio_112 | −0.106 | 0.146444 | −0.052 | −0.053 | 0.470 | 0.014 | 0.852 | 3-methylxanthine |
| bio_633 | −0.106 | 0.1481249 | −0.035 | −0.219 | 0.003 | 0.042 | 0.569 | X-12847 |
| bio_48 | −0.105 | 0.1498395 | 0.004 | −0.046 | 0.532 | 0.075 | 0.307 | 13-HODE + 9-HODE |
| bio_264 | −0.105 | 0.1500945 | −0.099 | 0.052 | 0.478 | −0.072 | 0.325 | gluconate |
| bio_653 | −0.104 | 0.1554994 | 0.068 | −0.107 | 0.142 | −0.178 | 0.014 | X-13848 |
| bio_513 | −0.104 | 0.1564588 | 0.008 | −0.096 | 0.193 | 0.117 | 0.111 | X-01911 |
| bio_448 | −0.104 | 0.1571279 | −0.119 | −0.168 | 0.021 | −0.064 | 0.380 | ribulose |
| bio_89 | −0.103 | 0.1604954 | −0.123 | −0.034 | 0.642 | −0.132 | 0.071 | 3-(cystein-S-yl)acetaminophen* |
| bio_239 | −0.102 | 0.164935 | 0.010 | −0.088 | 0.228 | 0.278 | 0.000 | eplandrosterone sulfate |
| bio_211 | −0.100 | 0.1740774 | −0.115 | −0.116 | 0.113 | 0.099 | 0.176 | cotinine |
| bio_141 | −0.099 | 0.176605 | −0.050 | 0.070 | 0.342 | 0.006 | 0.933 | 7-methylxanthine |
| bio_16 | −0.099 | 0.1769737 | −0.100 | 0.092 | 0.212 | −0.174 | 0.017 | 1- |
| | | | | | | | | docosahexaenoylglycerophospho- |
| | | | | | | | | ethanolamine* |
| bio_657 | −0.099 | 0.1777687 | −0.155 | −0.025 | 0.730 | −0.056 | 0.450 | X-14192 |
| bio_649 | −0.098 | 0.1803371 | 0.076 | −0.060 | 0.413 | 0.014 | 0.852 | X-13730 |
| bio_95 | −0.098 | 0.1831284 | 0.036 | −0.030 | 0.682 | 0.196 | 0.007 | 3-hydroxy-2-ethylpropionate |
| bio_75 | −0.096 | 0.1918858 | 0.048 | −0.052 | 0.482 | 0.166 | 0.022 | 2-linoleoylglycerophosphoethanol- |
| | | | | | | | | amine* |
| bio_295 | −0.095 | 0.1939523 | −0.171 | 0.029 | 0.691 | −0.167 | 0.022 | histidylalanine |
| bio_826 | −0.095 | 0.1941483 | −0.055 | 0.000 | 0.997 | 0.051 | 0.486 | X-19532 |
| bio_702 | −0.095 | 0.1958326 | 0.198 | −0.166 | 0.023 | 0.151 | 0.038 | X-16124 |
| bio_825 | −0.094 | 0.200387 | −0.082 | −0.212 | 0.003 | 0.053 | 0.469 | X-19455 |
| bio_362 | −0.093 | 0.2041809 | −0.001 | −0.214 | 0.003 | −0.017 | 0.816 | N-(2-furoyl)glycine |
| bio_804 | −0.092 | 0.2073801 | 0.049 | −0.184 | 0.011 | −0.016 | 0.831 | X-18945 |
| bio_410 | −0.092 | 0.2082778 | −0.051 | 0.154 | 0.035 | −0.033 | 0.656 | phenylacetate |
| bio_190 | −0.091 | 0.2136592 | 0.005 | 0.021 | 0.772 | 0.048 | 0.517 | caproate (6:0) |
| bio_792 | −0.090 | 0.217511 | −0.090 | 0.068 | 0.352 | −0.056 | 0.447 | X-18750 |
| bio_533 | −0.090 | 0.2192363 | −0.023 | 0.102 | 0.163 | −0.044 | 0.548 | X-11469 |
| bio_378 | −0.090 | 0.2220536 | −0.034 | 0.030 | 0.688 | −0.121 | 0.098 | N-methylhydantoin |
| bio_234 | −0.090 | 0.2221603 | −0.098 | −0.040 | 0.585 | 0.046 | 0.532 | dodecanedioate |
| bio_81 | −0.090 | 0.2222264 | 0.030 | −0.104 | 0.155 | 0.072 | 0.329 | 2-oleoylglycerophosphoethanol- |
| | | | | | | | | amine* |
| bio_158 | −0.088 | 0.2302051 | −0.046 | −0.132 | 0.070 | 0.236 | 0.001 | andro steroid monosulfate 2* |
| bio_67 | −0.087 | 0.2339467 | −0.125 | 0.029 | 0.695 | −0.108 | 0.141 | 2-hydroxyhippurate (salicylurate) |
| bio_733 | −0.087 | 0.237947 | −0.130 | 0.017 | 0.822 | −0.067 | 0.359 | X-17145 |
| bio_465 | −0.085 | 0.2450522 | −0.075 | 0.125 | 0.088 | −0.136 | 0.063 | stearoyl sphingomyelin |
| bio_13 | −0.085 | 0.2458374 | −0.129 | 0.069 | 0.349 | −0.009 | 0.904 | 1-arachidonylglycerol |
| bio_750 | −0.085 | 0.2477618 | 0.020 | 0.061 | 0.404 | −0.098 | 0.180 | X-17343 |
| bio_658 | −0.084 | 0.2506783 | −0.115 | −0.035 | 0.631 | −0.059 | 0.425 | X-14272 |
| bio_263 | −0.084 | 0.2528904 | −0.035 | −0.151 | 0.039 | 0.082 | 0.264 | gamma-tocopherol |
| bio_151 | −0.084 | 0.2533532 | −0.048 | 0.121 | 0.097 | 0.082 | 0.263 | allopurinol riboside |
| bio_459 | −0.084 | 0.2535799 | 0.039 | 0.016 | 0.829 | −0.036 | 0.623 | sorbitol |
| bio_800 | −0.082 | 0.2612227 | −0.032 | −0.001 | 0.990 | 0.145 | 0.046 | X-18913 |
| bio_555 | −0.082 | 0.2628058 | 0.030 | −0.002 | 0.981 | 0.054 | 0.466 | X-11805 |
| bio_65 | −0.082 | 0.2629695 | −0.007 | 0.107 | 0.145 | 0.173 | 0.018 | 2-hydroxydecanoic acid |
| bio_428 | −0.082 | 0.2644093 | −0.007 | −0.136 | 0.063 | 0.327 | 0.000 | pregnen-diol disulfate* |
| bio_805 | −0.081 | 0.2672602 | 0.057 | −0.036 | 0.626 | 0.097 | 0.184 | X-18946 |
| bio_166 | −0.081 | 0.2684686 | −0.125 | −0.061 | 0.405 | −0.095 | 0.195 | asparagylleucine |
| bio_562 | −0.080 | 0.2771791 | −0.007 | −0.106 | 0.149 | 0.205 | 0.005 | X-11852 |
| bio_474 | −0.080 | 0.278203 | −0.065 | −0.102 | 0.165 | −0.047 | 0.521 | taurocholenate sulfate* |
| bio_536 | −0.079 | 0.2821919 | −0.049 | 0.116 | 0.113 | 0.019 | 0.793 | X-11483 |
| bio_568 | −0.079 | 0.2822918 | 0.012 | 0.006 | 0.932 | 0.013 | 0.862 | X-12010 |
| bio_766 | −0.079 | 0.2846228 | −0.118 | −0.027 | 0.709 | −0.111 | 0.130 | X-17471 |
| bio_744 | −0.078 | 0.2867297 | −0.165 | 0.114 | 0.118 | 0.059 | 0.420 | X-17189 |
| bio_506 | −0.078 | 0.2876265 | −0.082 | −0.005 | 0.951 | −0.056 | 0.448 | valylvaline |
| bio_173 | −0.078 | 0.2886061 | −0.002 | −0.190 | 0.009 | 0.113 | 0.123 | benzoylecgonine |
| bio_569 | −0.078 | 0.2897334 | −0.160 | 0.056 | 0.443 | −0.089 | 0.224 | X-12027 |
| bio_149 | −0.074 | 0.3101965 | −0.077 | 0.019 | 0.799 | −0.088 | 0.230 | alanylleucine |
| bio_509 | −0.074 | 0.3104035 | −0.032 | 0.017 | 0.817 | 0.124 | 0.089 | xanthine |
| bio_8 | −0.073 | 0.3166922 | −0.004 | −0.094 | 0.198 | −0.131 | 0.073 | 1,7-dimethylurate |
| bio_505 | −0.072 | 0.3242292 | −0.112 | −0.030 | 0.680 | −0.074 | 0.312 | valylphenylalanine |
| bio_219 | −0.071 | 0.3327688 | 0.004 | −0.132 | 0.072 | −0.005 | 0.943 | delta-tocopherol |
| bio_198 | −0.071 | 0.3337583 | −0.047 | −0.028 | 0.699 | 0.050 | 0.496 | celecoxib |
| bio_200 | −0.070 | 0.3381101 | −0.008 | −0.022 | 0.765 | 0.028 | 0.704 | cholate |
| bio_503 | −0.070 | 0.3415623 | −0.049 | −0.028 | 0.702 | −0.039 | 0.600 | valylarginine |
| bio_668 | −0.069 | 0.3440535 | −0.106 | −0.064 | 0.381 | −0.202 | 0.005 | X-14632 |
| bio_703 | −0.067 | 0.3585538 | −0.033 | −0.003 | 0.965 | 0.087 | 0.234 | X-16125 |
| bio_451 | −0.067 | 0.3617666 | −0.099 | −0.016 | 0.826 | −0.107 | 0.145 | salicylate |
| bio_335 | −0.066 | 0.3673835 | −0.094 | 0.022 | 0.770 | −0.154 | 0.035 | leucylalanine |
| bio_20 | −0.066 | 0.3682698 | −0.096 | −0.036 | 0.622 | 0.004 | 0.955 | 1- |
| | | | | | | | | eicosatrienoylglycerophosphoeth- |
| | | | | | | | | anolamine* |
| bio_791 | −0.065 | 0.3727131 | −0.107 | 0.005 | 0.951 | 0.039 | 0.594 | X-18739 |
| bio_644 | −0.064 | 0.3824445 | −0.023 | −0.015 | 0.834 | −0.077 | 0.295 | X-13557 |
| bio_706 | −0.064 | 0.3833751 | 0.012 | −0.078 | 0.287 | 0.005 | 0.949 | X-16130 |
| bio_176 | −0.063 | 0.3915881 | 0.130 | −0.065 | 0.376 | 0.262 | 0.000 | beta-hydroxyisovalerate |
| bio_713 | −0.063 | 0.392761 | 0.060 | −0.113 | 0.123 | 0.003 | 0.966 | X-16288 |
| bio_458 | −0.062 | 0.4006038 | −0.052 | 0.020 | 0.784 | 0.070 | 0.341 | serylleucine |
| bio_317 | −0.060 | 0.4110917 | −0.106 | 0.053 | 0.475 | −0.128 | 0.080 | inosine |
| bio_689 | −0.060 | 0.4168395 | −0.130 | 0.139 | 0.058 | −0.052 | 0.479 | X-15664 |
| bio_34 | −0.059 | 0.4181974 | −0.195 | 0.030 | 0.688 | −0.132 | 0.071 | 1-palmitoylglycerophosphate |
| bio_318 | −0.055 | 0.4518135 | −0.034 | 0.056 | 0.443 | −0.063 | 0.389 | inositol 1-phosphate (I1P) |
| bio_814 | −0.055 | 0.4524404 | −0.020 | −0.117 | 0.110 | 0.032 | 0.665 | X-19166 |
| bio_164 | −0.054 | 0.4588111 | −0.133 | −0.035 | 0.630 | −0.127 | 0.082 | arginine |
| bio_718 | −0.054 | 0.463218 | −0.019 | −0.051 | 0.486 | 0.110 | 0.132 | X-16616 |
| bio_131 | −0.054 | 0.4633968 | 0.242 | 0.047 | 0.521 | 0.186 | 0.011 | 5-methyluridine (ribothymidine) |
| bio_731 | −0.054 | 0.4637158 | −0.136 | 0.162 | 0.026 | −0.057 | 0.434 | X-17137 |
| bio_300 | −0.054 | 0.465991 | 0.221 | 0.017 | 0.813 | −0.073 | 0.318 | homoveratric acid |
| bio_745 | −0.054 | 0.465991 | 0.221 | 0.017 | 0.813 | −0.073 | 0.318 | X-17192 |
| bio_728 | −0.053 | 0.4688951 | −0.017 | −0.060 | 0.411 | −0.039 | 0.600 | X-16947 |
| bio_761 | −0.052 | 0.4770267 | −0.031 | 0.010 | 0.894 | 0.080 | 0.277 | X-17443 |
| bio_354 | −0.052 | 0.4786391 | −0.134 | −0.022 | 0.761 | −0.146 | 0.046 | methyl-beta-glucopyranoside |
| bio_634 | −0.052 | 0.4826538 | −0.020 | −0.054 | 0.462 | 0.050 | 0.500 | X-12848 |
| bio_434 | −0.051 | 0.4881779 | −0.044 | −0.055 | 0.450 | 0.018 | 0.809 | pseudoephedrine |
| bio_610 | −0.051 | 0.4896517 | 0.018 | −0.110 | 0.132 | 0.139 | 0.056 | X-12730 |
| bio_233 | −0.049 | 0.5029163 | −0.132 | 0.146 | 0.045 | −0.100 | 0.171 | docosapentaenoate (n6 DPA; |
| | | | | | | | | 22:5n6) |
| bio_717 | −0.049 | 0.5029421 | −0.066 | −0.057 | 0.437 | −0.060 | 0.414 | X-16574 |
| bio_165 | −0.049 | 0.5078456 | −0.047 | −0.025 | 0.733 | −0.056 | 0.445 | asparagine |
| bio_436 | −0.047 | 0.526563 | −0.011 | 0.056 | 0.448 | −0.172 | 0.018 | pyridoxate |
| bio_674 | −0.045 | 0.5405672 | 0.156 | 0.027 | 0.711 | 0.257 | 0.000 | X-15382 |
| bio_420 | −0.044 | 0.5497651 | 0.002 | −0.097 | 0.186 | 0.109 | 0.137 | phenyllactate (PLA) |
| bio_306 | −0.043 | 0.5585169 | −0.090 | −0.011 | 0.879 | 0.029 | 0.692 | hydroxycotinine |
| bio_795 | −0.042 | 0.5631995 | 0.014 | 0.032 | 0.664 | 0.056 | 0.449 | X-18774 |
| bio_10 | −0.042 | 0.5709373 | −0.017 | −0.015 | 0.843 | −0.021 | 0.774 | 1- |
| | | | | | | | | arachidonoylglycerophosphoeth- |
| | | | | | | | | anolamine* |
| bio_764 | −0.041 | 0.5793816 | 0.037 | −0.066 | 0.372 | 0.024 | 0.747 | X-17454 |
| bio_787 | −0.041 | 0.5811439 | −0.027 | −0.021 | 0.778 | −0.053 | 0.470 | X-18482 |
| bio_619 | −0.040 | 0.5820255 | −0.177 | −0.004 | 0.952 | −0.091 | 0.216 | X-12798 |
| bio_793 | −0.040 | 0.5869207 | −0.132 | 0.127 | 0.083 | −0.002 | 0.982 | X-18752 |
| bio_333 | −0.038 | 0.6070536 | −0.094 | 0.059 | 0.418 | 0.060 | 0.415 | laurylcarnitine |
| bio_472 | −0.037 | 0.6148027 | −0.003 | 0.003 | 0.970 | −0.113 | 0.121 | taurochenodeoxycholate |
| bio_504 | −0.034 | 0.6401844 | −0.088 | −0.011 | 0.876 | −0.075 | 0.308 | valylhistidine |
| bio_528 | −0.033 | 0.6523458 | 0.076 | −0.144 | 0.048 | 0.333 | 0.000 | X-11440 |
| bio_751 | −0.031 | 0.6682892 | 0.031 | −0.098 | 0.183 | 0.003 | 0.963 | X-17347 |
| bio_433 | −0.031 | 0.6684902 | 0.064 | −0.008 | 0.915 | −0.010 | 0.890 | prostaglandin E2 |
| bio_502 | −0.031 | 0.6741698 | −0.062 | −0.004 | 0.959 | −0.031 | 0.676 | valylalanine |
| bio_140 | −0.031 | 0.67668 | −0.043 | −0.072 | 0.329 | 0.037 | 0.617 | 7-ketodeoxycholate |
| bio_307 | −0.031 | 0.6773673 | 0.048 | −0.035 | 0.633 | 0.048 | 0.516 | hyocholate |
| bio_332 | −0.030 | 0.6828745 | −0.057 | −0.034 | 0.643 | −0.029 | 0.694 | lauryl sulfate |
| bio_669 | −0.029 | 0.6888022 | −0.028 | −0.060 | 0.411 | −0.045 | 0.538 | X-14658 |
| bio_138 | −0.029 | 0.6908104 | −0.027 | 0.050 | 0.497 | 0.026 | 0.723 | 7-beta-hydroxycholesterol |
| bio_136 | −0.029 | 0.6912805 | 0.001 | −0.211 | 0.004 | −0.067 | 0.361 | 5alpha-pregnan-3beta,20alpha- |
| | | | | | | | | diol disulfate |
| bio_794 | −0.029 | 0.6924257 | −0.017 | 0.030 | 0.682 | 0.068 | 0.353 | X-18769 |
| bio_475 | −0.028 | 0.7002348 | −0.008 | 0.129 | 0.077 | −0.131 | 0.072 | taurodeoxycholate |
| bio_473 | −0.027 | 0.7122362 | −0.008 | 0.045 | 0.537 | −0.076 | 0.303 | taurocholate |
| bio_113 | −0.027 | 0.7133979 | −0.064 | 0.098 | 0.181 | −0.017 | 0.820 | 3-phenylpropionate |
| | | | | | | | | (hydrocinnamate) |
| bio_121 | −0.026 | 0.7263185 | 0.118 | −0.051 | 0.486 | 0.062 | 0.400 | 4-hydroxycyclohexylcarboxylic |
| | | | | | | | | acid |
| bio_412 | −0.025 | 0.7368116 | −0.069 | 0.088 | 0.230 | −0.048 | 0.512 | phenylalanine |
| bio_508 | −0.023 | 0.7552657 | −0.032 | 0.006 | 0.936 | 0.082 | 0.263 | warfarin |
| bio_338 | −0.022 | 0.7647154 | −0.101 | −0.035 | 0.634 | −0.111 | 0.130 | leucylphenylalanine |
| bio_170 | −0.022 | 0.7673155 | −0.029 | −0.023 | 0.756 | 0.050 | 0.496 | atenolol |
| bio_553 | −0.021 | 0.7739112 | 0.064 | 0.035 | 0.630 | 0.235 | 0.001 | X-11795 |
| bio_537 | −0.021 | 0.7788659 | 0.004 | −0.132 | 0.070 | 0.069 | 0.345 | X-11485 |
| bio_496 | −0.020 | 0.7875538 | −0.034 | 0.005 | 0.949 | −0.052 | 0.479 | tyrosyltryptophan |
| bio_29 | −0.018 | 0.8028561 | 0.012 | 0.052 | 0.481 | 0.046 | 0.529 | 1-oleoylglycerol (1-monoolein) |
| bio_416 | −0.018 | 0.8041694 | 0.018 | −0.018 | 0.809 | 0.009 | 0.903 | phenylalanylserine |
| bio_736 | −0.018 | 0.8044005 | −0.036 | −0.006 | 0.933 | −0.137 | 0.061 | X-17174 |
| bio_2 | −0.018 | 0.8056079 | 0.048 | 0.070 | 0.342 | −0.028 | 0.702 | 1,2-propanediol |
| bio_l | −0.018 | 0.8078943 | −0.010 | 0.003 | 0.969 | −0.050 | 0.494 | 1,2-dipalmitoylglycerol |
| bio_268 | −0.018 | 0.8101021 | −0.151 | 0.203 | 0.005 | 0.001 | 0.990 | glutamine |
| bio_296 | −0.017 | 0.8118539 | −0.089 | 0.001 | 0.993 | −0.139 | 0.056 | histidylphenylalanine |
| bio_614 | −0.017 | 0.8123812 | 0.165 | −0.143 | 0.051 | 0.011 | 0.879 | X-12740 |
| bio_191 | −0.017 | 0.8217283 | −0.097 | −0.069 | 0.348 | −0.065 | 0.374 | caprylate (8:0) |
| bio_636 | −0.016 | 0.8242181 | −0.029 | 0.185 | 0.011 | 0.057 | 0.440 | X-12851 |
| bio_425 | −0.014 | 0.8479646 | 0.017 | −0.109 | 0.138 | 0.021 | 0.775 | pipeline |
| bio_681 | −0.013 | 0.8616449 | −0.123 | −0.007 | 0.923 | −0.045 | 0.536 | X-15559 |
| bio_50 | −0.012 | 0.8742872 | −0.017 | 0.114 | 0.118 | 0.049 | 0.501 | 16-hydroxypalmitate |
| bio_115 | −0.012 | 0.8753917 | −0.056 | −0.085 | 0.246 | −0.147 | 0.044 | 4-acetamidophenol |
| bio_730 | −0.011 | 0.8842425 | 0.044 | −0.102 | 0.163 | −0.065 | 0.373 | X-17010 |
| bio_303 | −0.010 | 0.8900183 | −0.076 | −0.025 | 0.736 | −0.145 | 0.047 | HXGXA* |
| bio_712 | −0.010 | 0.8901975 | 0.095 | −0.068 | 0.355 | 0.065 | 0.374 | X-16245 |
| bio_711 | −0.010 | 0.8907077 | −0.010 | 0.065 | 0.372 | 0.008 | 0.914 | X-16235 |
| bio_84 | −0.009 | 0.9056482 | 0.076 | −0.016 | 0.829 | 0.019 | 0.796 | 2-piperidinone |
| bio_323 | −0.008 | 0.9141753 | 0.133 | −0.077 | 0.295 | 0.129 | 0.078 | isovalerylcarnitine |
| bio_829 | −0.007 | 0.9211932 | 0.001 | 0.042 | 0.563 | −0.003 | 0.967 | X-19779 |
| bio_5 | −0.007 | 0.926118 | −0.004 | 0.013 | 0.859 | −0.079 | 0.282 | 1,3-dipalmitoylglycerol |
| bio_347 | −0.007 | 0.9282962 | 0.018 | 0.214 | 0.003 | 0.159 | 0.030 | malate |
| bio_255 | −0.006 | 0.9320635 | −0.123 | 0.062 | 0.395 | 0.002 | 0.976 | gamma-glutamylglutamine |
| bio_584 | −0.006 | 0.9385134 | 0.003 | −0.022 | 0.766 | −0.010 | 0.893 | X-12205 |
| bio_341 | −0.005 | 0.9437284 | −0.008 | −0.062 | 0.401 | −0.035 | 0.636 | leukotriene B4 |
| bio_667 | −0.005 | 0.9462076 | 0.001 | −0.065 | 0.373 | −0.062 | 0.396 | X-14626 |
| bio_460 | −0.005 | 0.9472195 | −0.029 | 0.092 | 0.211 | 0.056 | 0.443 | sphingosine |
| bio_726 | −0.004 | 0.961179 | 0.067 | −0.168 | 0.021 | 0.014 | 0.850 | X-16940 |
| bio_284 | −0.003 | 0.9625073 | −0.033 | −0.047 | 0.519 | 0.069 | 0.349 | glycoursodeoxycholate |
| bio_682 | −0.001 | 0.9882637 | −0.078 | −0.007 | 0.926 | −0.030 | 0.683 | X-15563 |
| bio_779 | 0.000 | 0.9987965 | −0.089 | 0.020 | 0.789 | −0.157 | 0.032 | X-18039 |
| bio_376 | 0.000 | 0.9953307 | −0.129 | −0.043 | 0.563 | −0.169 | 0.020 | N-methyl proline |
| bio_174 | 0.001 | 0.9843853 | −0.028 | 0.004 | 0.962 | 0.087 | 0.234 | benzyl alcohol |
| bio_665 | 0.002 | 0.9826861 | −0.052 | 0.053 | 0.473 | 0.057 | 0.441 | X-14473 |
| bio_289 | 0.002 | 0.9745284 | 0.053 | 0.055 | 0.453 | 0.111 | 0.128 | heme |
| bio_159 | 0.003 | 0.9675729 | 0.070 | −0.111 | 0.128 | 0.281 | 0.000 | androsterone sulfate |
| bio_484 | 0.003 | 0.966144 | −0.033 | −0.061 | 0.409 | −0.021 | 0.778 | thymol sulfate |
| bio_827 | 0.004 | 0.9584086 | −0.082 | 0.024 | 0.745 | −0.122 | 0.095 | X-19574 |
| bio_541 | 0.006 | 0.9371107 | −0.007 | 0.065 | 0.374 | 0.160 | 0.028 | X-11538 |
| bio_423 | 0.006 | 0.9305269 | 0.002 | −0.065 | 0.379 | 0.056 | 0.449 | pimelate (heptanedioate) |
| bio_539 | 0.006 | 0.9297354 | −0.058 | 0.068 | 0.356 | 0.054 | 0.464 | X-11529 |
| bio_468 | 0.007 | 0.928351 | 0.057 | −0.054 | 0.466 | 0.170 | 0.019 | succinate |
| bio_129 | 0.007 | 0.9279369 | −0.026 | 0.008 | 0.913 | −0.011 | 0.880 | 5-HETE |
| bio_554 | 0.007 | 0.9246866 | −0.048 | −0.028 | 0.705 | −0.149 | 0.041 | X-11797 |
| bio_218 | 0.007 | 0.9240806 | 0.066 | −0.200 | 0.006 | 0.299 | 0.000 | dehydroisoandrosterone sulfate |
| | | | | | | | | (DHEA-S) |
| bio_708 | 0.008 | 0.9178502 | 0.044 | 0.087 | 0.236 | 0.028 | 0.706 | X-16134 |
| bio_327 | 0.008 | 0.9162537 | −0.021 | 0.085 | 0.245 | 0.192 | 0.008 | L-urobilin |
| bio_184 | 0.008 | 0.9092681 | −0.041 | −0.031 | 0.669 | −0.005 | 0.945 | bradykinin, des-arg(9) |
| bio_675 | 0.009 | 0.9030602 | 0.001 | 0.129 | 0.078 | 0.102 | 0.164 | X-15439 |
| bio_770 | 0.009 | 0.8996608 | 0.035 | −0.149 | 0.042 | 0.074 | 0.312 | X-17683 |
| bio_663 | 0.009 | 0.8993582 | −0.038 | 0.015 | 0.834 | 0.012 | 0.870 | X-14384 |
| bio_308 | 0.011 | 0.88008 | −0.093 | −0.003 | 0.965 | −0.157 | 0.031 | hypoxanthine |
| bio_507 | 0.012 | 0.8731424 | 0.028 | −0.049 | 0.505 | −0.073 | 0.318 | verapamil |
| bio_446 | 0.012 | 0.8674713 | 0.026 | 0.099 | 0.177 | 0.092 | 0.208 | ribitol |
| bio_464 | 0.014 | 0.8486131 | −0.020 | −0.039 | 0.597 | 0.077 | 0.294 | stearidonate (18:4n3) |
| bio_55 | 0.015 | 0.8353141 | 0.038 | −0.098 | 0.182 | 0.099 | 0.176 | 2-aminooctanoate |
| bio_734 | 0.015 | 0.8349885 | 0.006 | −0.045 | 0.540 | 0.061 | 0.404 | X-17146 |
| bio_477 | 0.016 | 0.8287452 | −0.014 | −0.032 | 0.662 | 0.094 | 0.198 | tetradecanedioate |
| bio_773 | 0.017 | 0.8199584 | −0.043 | −0.059 | 0.422 | 0.003 | 0.964 | X-17690 |
| bio_336 | 0.017 | 0.8145997 | −0.039 | 0.006 | 0.930 | −0.121 | 0.099 | leucylglycine |
| bio_747 | 0.021 | 0.773685 | 0.014 | −0.006 | 0.932 | 0.079 | 0.280 | X-17306 |
| bio_431 | 0.022 | 0.7675904 | −0.036 | 0.026 | 0.725 | 0.200 | 0.006 | proline |
| bio_156 | 0.022 | 0.7674424 | −0.002 | 0.027 | 0.714 | −0.221 | 0.002 | alpha-tocopherol |
| bio_688 | 0.023 | 0.7560426 | 0.015 | 0.062 | 0.398 | −0.009 | 0.899 | X-15650 |
| bio_59 | 0.023 | 0.7559994 | 0.009 | 0.049 | 0.507 | 0.006 | 0.933 | 2- |
| | | | | | | | | docosahexaenoylglycerophospho- |
| | | | | | | | | ethanolamine* |
| bio_321 | 0.023 | 0.755666 | −0.047 | 0.029 | 0.695 | −0.041 | 0.573 | isoleucylthreonine |
| bio_656 | 0.023 | 0.7521402 | −0.046 | 0.059 | 0.418 | −0.122 | 0.096 | X-14095 |
| bio_723 | 0.024 | 0.7389417 | −0.033 | −0.168 | 0.021 | −0.115 | 0.116 | X-16933 |
| bio_27 | 0.025 | 0.7309873 | 0.033 | −0.027 | 0.709 | −0.009 | 0.907 | 1-methylxanthine |
| bio_178 | 0.027 | 0.7145828 | 0.004 | −0.099 | 0.176 | 0.046 | 0.530 | beta-tocopherol |
| bio_635 | 0.028 | 0.7054765 | 0.012 | −0.035 | 0.635 | 0.110 | 0.135 | X-12850 |
| bio_799 | 0.029 | 0.6961934 | 0.047 | 0.033 | 0.656 | 0.076 | 0.301 | X-18908 |
| bio_168 | 0.029 | 0.6901025 | −0.007 | −0.084 | 0.254 | −0.005 | 0.942 | aspartylleucine |
| bio_476 | 0.029 | 0.6899827 | −0.031 | −0.002 | 0.983 | −0.094 | 0.199 | taurolithocholate 3-sulfate |
| bio_101 | 0.031 | 0.678004 | 0.064 | 0.054 | 0.466 | 0.112 | 0.126 | 3-hydroxypropanoate |
| bio_58 | 0.031 | 0.6732962 | 0.103 | −0.068 | 0.352 | −0.009 | 0.903 | 2-arachidonoylglycerophosphoeth- |
| | | | | | | | | anolamine* |
| bio_350 | 0.031 | 0.6725661 | 0.081 | −0.063 | 0.387 | 0.059 | 0.419 | mannose |
| bio_559 | 0.033 | 0.6582828 | 0.003 | −0.215 | 0.003 | −0.123 | 0.093 | X-11847 |
| bio_560 | 0.033 | 0.6529117 | 0.064 | −0.017 | 0.822 | −0.112 | 0.127 | X-11849 |
| bio_314 | 0.035 | 0.6292996 | −0.095 | 0.123 | 0.094 | −0.050 | 0.496 | indoleacrylate |
| bio_738 | 0.036 | 0.6238438 | 0.025 | 0.112 | 0.127 | 0.035 | 0.633 | X-17177 |
| bio_279 | 0.036 | 0.623251 | 0.047 | 0.044 | 0.550 | 0.028 | 0.703 | glycocholate |
| bio_33 | 0.038 | 0.6080143 | 0.022 | 0.037 | 0.612 | 0.060 | 0.412 | 1-palmitoylglycerol (1- |
| | | | | | | | | monopalmitin) |
| bio_96 | 0.039 | 0.5974789 | 0.071 | −0.055 | 0.453 | 0.085 | 0.247 | 3-hydroxybutyrate (BHBA) |
| bio_56 | 0.039 | 0.5974395 | −0.019 | 0.065 | 0.378 | 0.008 | 0.914 | 2-arachidonoyl glycerol |
| bio_236 | 0.039 | 0.5924959 | 0.054 | 0.109 | 0.136 | 0.052 | 0.480 | DSGEGDFXAEGGGVR* |
| bio_493 | 0.041 | 0.5760369 | −0.120 | 0.140 | 0.055 | −0.090 | 0.221 | tryptophan betaine |
| bio_527 | 0.041 | 0.5736756 | 0.118 | −0.104 | 0.157 | 0.096 | 0.191 | X-11438 |
| bio_118 | 0.042 | 0.5686321 | 0.081 | −0.158 | 0.030 | 0.278 | 0.000 | 4-androsten-3beta,17beta-diol |
| | | | | | | | | disulfate 1* |
| bio_40 | 0.042 | 0.566558 | −0.001 | −0.003 | 0.970 | −0.088 | 0.228 | 1-stearoylglycerol (1- |
| | | | | | | | | monostearin) |
| bio_183 | 0.042 | 0.5633529 | 0.013 | −0.025 | 0.736 | −0.021 | 0.771 | bisphenol A monosulfate |
| bio_304 | 0.043 | 0.5591819 | 0.006 | −0.038 | 0.610 | −0.039 | 0.598 | hydrochlorothiazide |
| bio_563 | 0.044 | 0.5533572 | 0.048 | −0.022 | 0.768 | −0.092 | 0.209 | X-11858 |
| bio_291 | 0.044 | 0.5519292 | 0.059 | −0.025 | 0.734 | 0.099 | 0.176 | hexadecanedioate |
| bio_673 | 0.044 | 0.5506061 | 0.087 | 0.015 | 0.835 | 0.160 | 0.028 | X-15220 |
| bio_547 | 0.044 | 0.5505292 | 0.126 | 0.082 | 0.263 | 0.023 | 0.751 | X-11632 |
| bio_344 | 0.045 | 0.5426202 | 0.013 | 0.062 | 0.398 | −0.014 | 0.844 | linolenate [alpha or gamma; |
| | | | | | | | | (18:3n3 or 6)] |
| bio_680 | 0.045 | 0.5394906 | 0.020 | −0.013 | 0.863 | 0.015 | 0.837 | X-15558 |
| bio_273 | 0.046 | 0.5280607 | −0.054 | 0.077 | 0.292 | −0.119 | 0.105 | glycerol |
| bio_213 | 0.047 | 0.51977 | 0.084 | 0.027 | 0.716 | −0.291 | 0.000 | creatine |
| bio_600 | 0.048 | 0.516684 | −0.066 | −0.057 | 0.441 | −0.117 | 0.110 | X-12524 |
| bio_592 | 0.048 | 0.5142908 | 0.022 | −0.049 | 0.506 | −0.036 | 0.621 | X-12306 |
| bio_769 | 0.049 | 0.504518 | −0.001 | −0.209 | 0.004 | −0.096 | 0.192 | X-17655 |
| bio_361 | 0.050 | 0.5000343 | −0.070 | 0.021 | 0.771 | −0.112 | 0.124 | myristoleate (14:1n5) |
| bio_259 | 0.050 | 0.4989409 | 0.120 | −0.211 | 0.004 | 0.160 | 0.028 | gamma-glutamylphenylalanine |
| bio_424 | 0.051 | 0.4845103 | 0.041 | 0.003 | 0.963 | 0.108 | 0.140 | pipecolate |
| bio_339 | 0.052 | 0.4754465 | −0.031 | 0.041 | 0.574 | −0.033 | 0.657 | leucylthreonine |
| bio_342 | 0.055 | 0.456858 | 0.049 | 0.101 | 0.168 | 0.050 | 0.496 | linamarin |
| bio_617 | 0.056 | 0.4443059 | 0.143 | −0.021 | 0.770 | 0.061 | 0.404 | X-12748 |
| bio_14 | 0.056 | 0.444079 | 0.001 | 0.027 | 0.713 | 0.012 | 0.871 | 1-dihomo- |
| | | | | | | | | linoleoylglycerophosphocholine |
| | | | | | | | | (20:2n6)* |
| bio_210 | 0.057 | 0.4411152 | 0.032 | 0.035 | 0.635 | 0.009 | 0.906 | cortisone |
| bio_535 | 0.057 | 0.4382153 | 0.049 | −0.093 | 0.205 | 0.011 | 0.883 | X-11478 |
| bio_707 | 0.058 | 0.4263411 | 0.028 | −0.019 | 0.801 | −0.104 | 0.157 | X-16132 |
| bio_741 | 0.059 | 0.4229764 | −0.017 | 0.071 | 0.334 | −0.051 | 0.487 | X-17183 |
| bio_780 | 0.059 | 0.4225445 | 0.056 | 0.046 | 0.530 | 0.100 | 0.171 | X-18241 |
| bio_265 | 0.059 | 0.4178302 | 0.175 | −0.012 | 0.869 | 0.063 | 0.389 | glucose |
| bio_171 | 0.060 | 0.4158543 | −0.008 | 0.082 | 0.266 | 0.001 | 0.985 | azelate (nonanedioate) |
| bio_135 | 0.060 | 0.4115281 | 0.108 | −0.114 | 0.120 | 0.398 | 0.000 | 5alpha-androstan-3beta,17beta- |
| | | | | | | | | diol disulfate |
| bio_494 | 0.061 | 0.4077348 | 0.067 | −0.096 | 0.190 | 0.061 | 0.408 | tryptophylglutamate |
| bio_402 | 0.061 | 0.4025866 | −0.038 | 0.060 | 0.416 | −0.105 | 0.153 | palmitoleate (16:1n7) |
| bio_297 | 0.062 | 0.4020957 | 0.009 | −0.079 | 0.284 | −0.011 | 0.884 | histidyltryptophan |
| bio_408 | 0.062 | 0.4006762 | 0.040 | −0.023 | 0.759 | −0.003 | 0.969 | pentadecanoate (15:0) |
| bio_148 | 0.062 | 0.3973425 | −0.013 | 0.096 | 0.190 | 0.017 | 0.820 | alanine |
| bio_237 | 0.063 | 0.3927451 | −0.027 | 0.043 | 0.555 | 0.030 | 0.685 | eicosapentaenoate (EPA; 20:5n3) |
| bio_337 | 0.064 | 0.3867746 | −0.012 | −0.075 | 0.305 | −0.090 | 0.222 | leucylleucine |
| bio_705 | 0.064 | 0.3816099 | 0.001 | 0.087 | 0.236 | −0.036 | 0.621 | X-16129 |
| bio_331 | 0.065 | 0.3737135 | −0.050 | 0.015 | 0.842 | −0.084 | 0.251 | laurate (12:0) |
| bio_30 | 0.067 | 0.3588946 | 0.074 | −0.040 | 0.584 | 0.040 | 0.591 | 1-oleoylglycerophosphocholine |
| | | | | | | | | (18:1) |
| bio_551 | 0.067 | 0.3587058 | −0.017 | −0.074 | 0.316 | −0.067 | 0.364 | X-11792 |
| bio_407 | 0.068 | 0.356812 | 0.069 | 0.056 | 0.446 | 0.050 | 0.493 | pelargonate (9:0) |
| bio_660 | 0.068 | 0.3560344 | −0.004 | −0.013 | 0.865 | −0.010 | 0.889 | X-14314 |
| bio_25 | 0.068 | 0.3548062 | 0.118 | −0.030 | 0.686 | 0.079 | 0.279 | 1- |
| | | | | | | | | margaroylglycerophosphocholine |
| | | | | | | | | (17:0) |
| bio_278 | 0.068 | 0.3511494 | 0.056 | 0.002 | 0.979 | 0.027 | 0.710 | glycochenodeoxycholate |
| bio_641 | 0.069 | 0.3502281 | 0.010 | 0.063 | 0.391 | 0.007 | 0.929 | X-13452 |
| bio_343 | 0.069 | 0.3486423 | 0.012 | 0.063 | 0.394 | −0.062 | 0.396 | linoleate (18:2n6) |
| bio_281 | 0.069 | 0.3471267 | −0.009 | 0.138 | 0.059 | 0.016 | 0.832 | glycodeoxycholate |
| bio_491 | 0.070 | 0.3416729 | 0.018 | −0.121 | 0.098 | −0.149 | 0.041 | trizma acetate |
| bio_715 | 0.070 | 0.3401433 | 0.021 | −0.052 | 0.476 | −0.101 | 0.169 | X-16439 |
| bio_700 | 0.070 | 0.336854 | 0.119 | −0.020 | 0.782 | 0.161 | 0.027 | X-16094 |
| bio_391 | 0.071 | 0.3323982 | 0.013 | 0.067 | 0.364 | 0.175 | 0.016 | octadecanedioate |
| bio_777 | 0.072 | 0.3295881 | 0.039 | −0.011 | 0.882 | 0.094 | 0.198 | X-17856 |
| bio_457 | 0.072 | 0.3242035 | −0.095 | −0.036 | 0.626 | −0.126 | 0.084 | serotonin (SHT) |
| bio_66 | 0.073 | 0.3199346 | 0.015 | 0.046 | 0.535 | −0.018 | 0.811 | 2-hydroxyglutarate |
| bio_709 | 0.074 | 0.3104475 | 0.046 | 0.064 | 0.384 | −0.027 | 0.716 | X-16135 |
| bio_676 | 0.075 | 0.3094161 | 0.252 | −0.073 | 0.320 | 0.273 | 0.000 | X-15484 |
| bio_270 | 0.075 | 0.3046505 | 0.065 | 0.044 | 0.546 | 0.004 | 0.961 | glutarate (pentanedioate) |
| bio_221 | 0.076 | 0.2985684 | 0.054 | 0.078 | 0.289 | 0.082 | 0.261 | deoxycholate |
| bio_497 | 0.078 | 0.2899621 | 0.008 | 0.074 | 0.316 | −0.007 | 0.922 | undecanedioate |
| bio_226 | 0.078 | 0.2881276 | 0.051 | 0.009 | 0.906 | −0.101 | 0.166 | dihydroorotate |
| bio_206 | 0.078 | 0.2869965 | −0.043 | 0.030 | 0.685 | −0.059 | 0.421 | cis-vaccenate (18:1n7) |
| bio_146 | 0.078 | 0.2866589 | 0.110 | 0.002 | 0.981 | 0.091 | 0.215 | adipate |
| bio_41 | 0.078 | 0.2866021 | 0.114 | −0.035 | 0.638 | 0.072 | 0.326 | 1-stearoylglycerophosphocholine |
| | | | | | | | | (18:0) |
| bio_394 | 0.079 | 0.2828998 | 0.033 | 0.063 | 0.389 | 0.118 | 0.107 | oleoylcarnitine |
| bio_79 | 0.080 | 0.2776799 | 0.106 | 0.036 | 0.622 | 0.057 | 0.435 | 2-oleoylglycerol (2-monoolein) |
| bio_778 | 0.080 | 0.2776457 | 0.009 | 0.141 | 0.054 | −0.117 | 0.110 | X-17969 |
| bio_189 | 0.080 | 0.2756049 | −0.028 | −0.026 | 0.726 | −0.031 | 0.676 | caprate (10:0) |
| bio_406 | 0.083 | 0.2605876 | 0.076 | −0.076 | 0.298 | −0.014 | 0.852 | paraxanthine |
| bio_169 | 0.085 | 0.24436 | 0.116 | −0.113 | 0.123 | 0.073 | 0.317 | aspartylphenylalanine |
| bio_6 | 0.086 | 0.2418458 | −0.124 | −0.026 | 0.719 | 0.115 | 0.118 | 1,5-anhydroglucitol (1,5-AG) |
| bio_329 | 0.087 | 0.237383 | 0.089 | 0.085 | 0.246 | 0.050 | 0.496 | lansoprazole |
| bio_280 | 0.092 | 0.2091266 | 0.004 | −0.058 | 0.426 | 0.170 | 0.020 | glycocholenate sulfate* |
| bio_449 | 0.092 | 0.2072285 | 0.008 | 0.056 | 0.446 | 0.099 | 0.176 | S-methylcysteine |
| bio_23 | 0.094 | 0.19913 | 0.109 | −0.075 | 0.309 | 0.106 | 0.147 | 1- |
| | | | | | | | | linoleoylglycerophosphocholine |
| | | | | | | | | (18:2n6) |
| bio_455 | 0.096 | 0.1889035 | 0.022 | 0.038 | 0.602 | 0.061 | 0.409 | sebacate (decanedioate) |
| bio_128 | 0.097 | 0.1867907 | −0.028 | 0.076 | 0.302 | −0.080 | 0.277 | 5-dodecenoate (12:1n7) |
| bio_467 | 0.097 | 0.1859878 | 0.042 | 0.092 | 0.210 | 0.060 | 0.414 | suberate (octanedioate) |
| bio_759 | 0.097 | 0.1849759 | 0.028 | 0.092 | 0.211 | 0.004 | 0.956 | X-17438 |
| bio_466 | 0.098 | 0.1808646 | 0.052 | 0.165 | 0.023 | 0.212 | 0.003 | stearoylcarnitine |
| bio_231 | 0.098 | 0.1805909 | −0.010 | 0.221 | 0.002 | −0.158 | 0.030 | docosahexaenoate (DHA; |
| | | | | | | | | 22:6n3) |
| bio_316 | 0.099 | 0.1782883 | 0.071 | 0.033 | 0.657 | −0.085 | 0.246 | indolepropionate |
| bio_35 | 0.100 | 0.173279 | 0.090 | −0.024 | 0.741 | 0.048 | 0.510 | 1- |
| | | | | | | | | palmitoylglycerophosphocholine |
| | | | | | | | | (16:0) |
| bio_478 | 0.102 | 0.1646311 | 0.094 | −0.055 | 0.452 | 0.017 | 0.817 | theobromine |
| bio_456 | 0.102 | 0.1618839 | −0.070 | 0.024 | 0.740 | −0.129 | 0.078 | serine |
| bio_540 | 0.103 | 0.1602429 | 0.013 | −0.059 | 0.419 | 0.089 | 0.226 | X-11537 |
| bio_243 | 0.103 | 0.159879 | −0.017 | 0.023 | 0.757 | −0.224 | 0.002 | erythrulose |
| bio_155 | 0.103 | 0.1593735 | 0.157 | −0.119 | 0.103 | 0.184 | 0.011 | alpha-ketoglutarate |
| bio_240 | 0.104 | 0.1565773 | 0.095 | −0.093 | 0.204 | 0.123 | 0.092 | erucate (22:1n9) |
| bio_80 | 0.107 | 0.1458936 | 0.088 | −0.055 | 0.450 | 0.123 | 0.093 | 2-oleoylglycerophosphocholine* |
| bio_282 | 0.107 | 0.1453374 | −0.011 | 0.064 | 0.385 | −0.040 | 0.586 | glycolate (hydroxyacetate) |
| bio_811 | 0.107 | 0.1424021 | 0.075 | 0.035 | 0.631 | 0.198 | 0.006 | X-19137 |
| bio_39 | 0.108 | 0.1416018 | 0.061 | −0.040 | 0.582 | −0.031 | 0.673 | 1- |
| | | | | | | | | pentadecanoylglycerophospho- |
| | | | | | | | | choline (15:0)* |
| bio_639 | 0.108 | 0.1409943 | 0.033 | 0.031 | 0.674 | 0.128 | 0.081 | X-13429 |
| bio_51 | 0.109 | 0.1379754 | 0.075 | −0.069 | 0.346 | 0.084 | 0.251 | 17-methylstearate |
| bio_15 | 0.110 | 0.1330774 | 0.062 | 0.023 | 0.751 | −0.020 | 0.788 | 1- |
| | | | | | | | | docosahexaenoylglycerophospho- |
| | | | | | | | | choline (22:6n3)* |
| bio_132 | 0.112 | 0.1265501 | 0.055 | 0.108 | 0.139 | 0.139 | 0.058 | 5-oxoproline |
| bio_137 | 0.114 | 0.118525 | 0.000 | 0.134 | 0.067 | 0.189 | 0.009 | 7-alpha-hydroxy-3-oxo-4- |
| | | | | | | | | cholestenoate (7-Hoca) |
| bio_181 | 0.114 | 0.1182317 | 0.057 | −0.020 | 0.788 | 0.112 | 0.126 | bilirubin (Z,Z) |
| bio_133 | 0.116 | 0.1142729 | 0.165 | −0.041 | 0.573 | 0.368 | 0.000 | 5alpha-androstan- |
| | | | | | | | | 3alpha,17beta-diol disulfate |
| bio_302 | 0.121 | 0.0975342 | −0.034 | 0.055 | 0.453 | −0.117 | 0.111 | HWESASXX* |
| bio_330 | 0.122 | 0.0941326 | 0.143 | −0.137 | 0.060 | 0.084 | 0.252 | lathosterol |
| bio_815 | 0.124 | 0.090072 | 0.124 | −0.049 | 0.506 | −0.013 | 0.862 | X-19302 |
| bio_360 | 0.125 | 0.0883875 | 0.011 | 0.036 | 0.628 | −0.026 | 0.724 | myristate (14:0) |
| bio_85 | 0.125 | 0.087229 | 0.158 | −0.052 | 0.475 | 0.115 | 0.117 | 2- |
| | | | | | | | | stearoylglycerophosphocholine* |
| bio_62 | 0.126 | 0.085741 | 0.107 | −0.025 | 0.736 | 0.177 | 0.015 | 2-hydroxy-3-methylvalerate |
| bio_32 | 0.126 | 0.0847135 | 0.080 | −0.010 | 0.887 | 0.028 | 0.704 | 1- |
| | | | | | | | | palmitoleoylglycerophosphocholine |
| | | | | | | | | (16:1)* |
| bio_45 | 0.128 | 0.0802077 | 0.066 | 0.040 | 0.589 | −0.014 | 0.852 | 10-heptadecenoate (17:1n7) |
| bio_71 | 0.128 | 0.0796824 | −0.034 | 0.100 | 0.171 | −0.035 | 0.632 | 2-hydroxypalmitate |
| bio_760 | 0.131 | 0.0741002 | 0.028 | 0.077 | 0.296 | 0.069 | 0.348 | X-17441 |
| bio_404 | 0.131 | 0.0735695 | 0.040 | −0.009 | 0.903 | 0.144 | 0.049 | palmitoylcarnitine |
| bio_37 | 0.131 | 0.0730348 | −0.049 | 0.016 | 0.828 | −0.023 | 0.753 | 1- |
| | | | | | | | | palmitoylglycerophosphoinositol* |
| bio_46 | 0.131 | 0.0728979 | 0.056 | 0.046 | 0.533 | 0.006 | 0.938 | 10-nonadecenoate (19:1n9) |
| bio_328 | 0.132 | 0.0701298 | 0.108 | 0.138 | 0.059 | 0.129 | 0.078 | lactate |
| bio_393 | 0.133 | 0.06837 | 0.083 | 0.066 | 0.366 | −0.009 | 0.901 | oleate (18:1n9) |
| bio_154 | 0.134 | 0.0660044 | 0.107 | −0.042 | 0.569 | 0.158 | 0.030 | alpha-hydroxyisovalerate |
| bio_53 | 0.136 | 0.0627417 | 0.160 | 0.070 | 0.340 | 0.144 | 0.049 | 2-aminobutyrate |
| bio_301 | 0.138 | 0.0590755 | 0.104 | −0.045 | 0.544 | 0.023 | 0.759 | HWESASLLR |
| bio_322 | 0.138 | 0.0586226 | 0.135 | −0.099 | 0.176 | 0.060 | 0.417 | isovalerate |
| bio_351 | 0.139 | 0.0564062 | 0.078 | 0.072 | 0.329 | 0.047 | 0.525 | margarate (17:0) |
| bio_230 | 0.141 | 0.0542512 | 0.086 | 0.053 | 0.468 | 0.053 | 0.467 | docosadienoate (22:2n6) |
| bio_463 | 0.141 | 0.0542196 | 0.034 | 0.140 | 0.055 | −0.006 | 0.939 | stearate (18:0) |
| bio_98 | 0.142 | 0.0522544 | 0.027 | 0.101 | 0.166 | 0.022 | 0.770 | 3-hydroxydecanoate |
| bio_272 | 0.144 | 0.0491261 | 0.062 | 0.057 | 0.437 | −0.096 | 0.192 | glycerate |
| bio_74 | 0.144 | 0.04843 | 0.144 | −0.100 | 0.171 | 0.122 | 0.097 | 2-linoleoylglycerophosphocholine* |
| bio_147 | 0.144 | 0.047927 | 0.138 | 0.063 | 0.388 | 0.057 | 0.440 | ADSGEGDFXAEGGGVR* |
| bio_100 | 0.146 | 0.0452548 | 0.284 | −0.088 | 0.228 | 0.190 | 0.009 | 3-hydroxyisobutyrate |
| bio_542 | 0.146 | 0.0450618 | 0.030 | −0.005 | 0.951 | 0.128 | 0.080 | X-11540 |
| bio_238 | 0.147 | 0.0443221 | 0.098 | 0.065 | 0.378 | 0.012 | 0.871 | eicosenoate (20:1n9 or 11) |
| bio_725 | 0.147 | 0.0442789 | −0.003 | 0.031 | 0.677 | 0.087 | 0.238 | X-16935 |
| bio_21 | 0.147 | 0.0435655 | 0.078 | −0.044 | 0.547 | 0.091 | 0.215 | 1- |
| | | | | | | | | linolenoylglycerophosphocholine |
| | | | | | | | | (18:3n3)* |
| bio_260 | 0.148 | 0.0421686 | 0.167 | −0.271 | 0.000 | 0.136 | 0.062 | gamma-glutamylthreonine* |
| bio_543 | 0.150 | 0.0395328 | 0.077 | 0.128 | 0.079 | 0.078 | 0.289 | X-11541 |
| bio_662 | 0.153 | 0.0358155 | 0.056 | 0.056 | 0.449 | 0.011 | 0.879 | X-14364 |
| bio_283 | 0.154 | 0.0347063 | 0.049 | 0.131 | 0.073 | 0.057 | 0.438 | glycolithocholate sulfate* |
| bio_224 | 0.154 | 0.0346452 | 0.085 | 0.102 | 0.163 | 0.005 | 0.947 | dihomo-linoleate (20:2n6) |
| bio_57 | 0.158 | 0.0306321 | 0.212 | −0.077 | 0.291 | 0.042 | 0.564 | 2-arachidonoylglycerophosphocholine* |
| bio_439 | 0.158 | 0.0298045 | 0.028 | −0.075 | 0.310 | −0.122 | 0.095 | pyroglutamylglycine |
| bio_572 | 0.160 | 0.0279673 | 0.081 | 0.010 | 0.891 | 0.118 | 0.106 | X-12063 |
| bio_479 | 0.160 | 0.0279085 | 0.094 | −0.040 | 0.586 | −0.109 | 0.137 | theophylline |
| bio_225 | 0.160 | 0.0278272 | 0.030 | 0.117 | 0.109 | 0.035 | 0.636 | dihomo-linolenate (20:3n3 or n6) |
| bio_187 | 0.162 | 0.0263401 | 0.133 | −0.066 | 0.367 | −0.106 | 0.148 | caffeine |
| bio_82 | 0.163 | 0.0252926 | 0.163 | −0.084 | 0.251 | 0.059 | 0.423 | 2- |
| | | | | | | | | palmitoylglycerophosphocholine* |
| bio_524 | 0.163 | 0.0251217 | 0.051 | −0.024 | 0.741 | −0.097 | 0.185 | X-11381 |
| bio_796 | 0.165 | 0.0236642 | 0.134 | −0.035 | 0.634 | −0.052 | 0.477 | X-18779 |
| bio_12 | 0.166 | 0.0230605 | 0.034 | −0.106 | 0.149 | −0.061 | 0.404 | 1-arachidonoylglycercophosphate |
| bio_802 | 0.166 | 0.0229133 | 0.078 | 0.037 | 0.612 | 0.023 | 0.755 | X-18928 |
| bio_256 | 0.166 | 0.0225436 | 0.184 | −0.199 | 0.006 | 0.221 | 0.002 | gamma-glutamylisoleucine* |
| bio_415 | 0.167 | 0.0218388 | 0.048 | −0.080 | 0.275 | 0.002 | 0.980 | phenylalanylphenylalanine |
| bio_17 | 0.168 | 0.0214292 | 0.108 | −0.036 | 0.626 | 0.213 | 0.003 | 1- |
| | | | | | | | | docosapentaenoylglycerophospho- |
| | | | | | | | | choline (22:5)* |
| bio_19 | 0.168 | 0.0212869 | 0.127 | −0.121 | 0.098 | 0.074 | 0.313 | 1- |
| | | | | | | | | eicosatrienoylglycerophospho- |
| | | | | | | | | choline (20:3)* |
| bio_163 | 0.169 | 0.0200055 | 0.016 | 0.078 | 0.288 | −0.045 | 0.537 | arachidonate (20:4n6) |
| bio_659 | 0.170 | 0.0194555 | 0.053 | 0.057 | 0.441 | 0.038 | 0.605 | X-14302 |
| bio_216 | 0.170 | 0.0194024 | 0.119 | 0.079 | 0.283 | 0.076 | 0.302 | cyclo(leu-pro) |
| bio_72 | 0.170 | 0.0193198 | −0.058 | 0.066 | 0.366 | −0.093 | 0.207 | 2-hydroxystearate |
| bio_254 | 0.173 | 0.0175269 | 0.134 | −0.162 | 0.026 | 0.112 | 0.127 | gamma-glutamylglutamate |
| bio_388 | 0.175 | 0.0163961 | 0.078 | 0.036 | 0.622 | 0.045 | 0.537 | nonadecanoate (19:0) |
| bio_400 | 0.179 | 0.0140485 | 0.084 | 0.069 | 0.345 | −0.022 | 0.761 | palmitate (16:0) |
| bio_49 | 0.180 | 0.0134731 | 0.139 | −0.103 | 0.161 | 0.068 | 0.352 | 15-methylpalmitate (isobar with |
| | | | | | | | | 2-methylpalmitate) |
| bio_417 | 0.182 | 0.0120803 | 0.062 | 0.020 | 0.784 | 0.062 | 0.401 | phenylalanyltryptophan |
| bio_44 | 0.183 | 0.0118093 | 0.217 | −0.025 | 0.730 | 0.140 | 0.055 | 1- |
| | | | | | | | | stearoylplasmenylethanolamine* |
| bio_414 | 0.186 | 0.0106173 | 0.180 | −0.112 | 0.127 | 0.062 | 0.401 | phenylalanylleucine |
| bio_320 | 0.186 | 0.0105992 | 0.199 | −0.039 | 0.593 | 0.336 | 0.000 | isoleucine |
| bio_267 | 0.188 | 0.00947 | 0.118 | −0.136 | 0.063 | 0.123 | 0.093 | glutamate |
| bio_544 | 0.194 | 0.0076684 | 0.057 | −0.060 | 0.411 | −0.089 | 0.223 | X-11550 |
| bio_258 | 0.194 | 0.0075373 | 0.093 | −0.131 | 0.072 | 0.128 | 0.079 | gamma-glutamylmethionine |
| bio_704 | 0.195 | 0.0071019 | 0.139 | −0.173 | 0.018 | −0.127 | 0.082 | X-16128 |
| bio_781 | 0.196 | 0.0069195 | −0.006 | 0.199 | 0.006 | −0.040 | 0.582 | X-18249 |
| bio_9 | 0.200 | 0.0058231 | 0.155 | −0.035 | 0.638 | 0.052 | 0.479 | 1- |
| | | | | | | | | arachidonoylglycerophosphocholine |
| | | | | | | | | (20:4n6)* |
| bio_346 | 0.201 | 0.0056827 | 0.118 | −0.036 | 0.624 | 0.005 | 0.946 | lysine |
| bio_43 | 0.201 | 0.0055696 | −0.008 | 0.017 | 0.822 | −0.069 | 0.350 | 1- |
| | | | | | | | | stearoylglycerophosphoinositol |
| bio_38 | 0.203 | 0.0052014 | 0.185 | 0.018 | 0.807 | 0.049 | 0.502 | 1- |
| | | | | | | | | palmitoylplasmenylethanolamine* |
| bio_727 | 0.203 | 0.0050039 | 0.078 | 0.066 | 0.371 | 0.205 | 0.005 | X-16946 |
| bio_521 | 0.204 | 0.0049504 | −0.083 | 0.141 | 0.053 | −0.093 | 0.206 | X-11315 |
| bio_670 | 0.206 | 0.0043831 | 0.179 | −0.075 | 0.309 | −0.014 | 0.849 | X-14939 |
| bio_798 | 0.208 | 0.0040136 | 0.103 | 0.108 | 0.141 | 0.095 | 0.195 | X-18898 |
| bio_801 | 0.209 | 0.0038865 | 0.046 | 0.166 | 0.023 | 0.122 | 0.096 | X-18914 |
| bio_86 | 0.214 | 0.0031458 | 0.044 | −0.005 | 0.941 | −0.063 | 0.391 | 2- |
| | | | | | | | | stearoylglycerophosphoinositol* |
| bio_28 | 0.217 | 0.002736 | 0.162 | −0.120 | 0.101 | 0.006 | 0.938 | 1- |
| | | | | | | | | myristoylglycerophosphocholine |
| | | | | | | | | (14:0) |
| bio_60 | 0.219 | 0.0024884 | −0.014 | −0.010 | 0.887 | −0.125 | 0.087 | 2-ethylhexanoate |
| bio_182 | 0.221 | 0.0022602 | 0.148 | 0.107 | 0.143 | 0.231 | 0.001 | biliverdin |
| bio_18 | 0.221 | 0.0022481 | 0.151 | 0.051 | 0.491 | 0.041 | 0.575 | 1- |
| | | | | | | | | eicosapentaenoylglycerophos- |
| | | | | | | | | phocholine (20:5n3)* |
| bio_642 | 0.224 | 0.0019768 | 0.161 | 0.000 | 0.998 | −0.092 | 0.208 | X-13483 |
| bio_803 | 0.227 | 0.0016919 | 0.181 | 0.137 | 0.060 | 0.236 | 0.001 | X-18929 |
| bio_387 | 0.230 | 0.0014521 | 0.230 | 0.084 | 0.250 | 0.057 | 0.440 | nicotinamide |
| bio_401 | 0.232 | 0.0013312 | 0.062 | 0.115 | 0.115 | −0.027 | 0.710 | palmitate, methyl ester |
| bio_353 | 0.232 | 0.0013164 | 0.181 | 0.021 | 0.779 | 0.125 | 0.089 | methionine |
| bio_598 | 0.232 | 0.0012875 | 0.275 | −0.015 | 0.835 | 0.062 | 0.400 | X-12462 |
| bio_108 | 0.234 | 0.0012061 | 0.128 | 0.043 | 0.560 | 0.083 | 0.260 | 3-methyl-2-oxobutyrate |
| bio_520 | 0.235 | 0.0011467 | 0.054 | 0.034 | 0.648 | 0.059 | 0.423 | X-11308 |
| bio_724 | 0.242 | 0.0008035 | 0.254 | 0.106 | 0.147 | 0.227 | 0.002 | X-16934 |
| bio_232 | 0.243 | 0.0007633 | 0.189 | 0.082 | 0.262 | 0.071 | 0.333 | docosapentaenoate (n3 DPA; |
| | | | | | | | | 22:5n3) |
| bio_488 | 0.243 | 0.0007427 | 0.227 | 0.028 | 0.701 | 0.105 | 0.153 | trans-urocanate |
| bio_334 | 0.246 | 0.0006486 | 0.194 | −0.045 | 0.538 | 0.264 | 0.000 | leucine |
| bio_153 | 0.246 | 0.0006327 | 0.251 | 0.038 | 0.608 | 0.215 | 0.003 | alpha-hydroxyisocaproate |
| bio_275 | 0.247 | 0.0006061 | 0.099 | −0.002 | 0.983 | −0.012 | 0.873 | glycerol 3-phosphate (G3P) |
| bio_180 | 0.249 | 0.00054 | 0.148 | 0.015 | 0.843 | 0.192 | 0.008 | bilirubin (E,E)* |
| bio_257 | 0.250 | 0.0005201 | 0.210 | −0.208 | 0.004 | 0.202 | 0.005 | gamma-glutamylleucine |
| bio_11 | 0.251 | 0.0004794 | 0.051 | −0.034 | 0.640 | 0.011 | 0.878 | 1- |
| | | | | | | | | arachidonoylglycerophosphoino- |
| | | | | | | | | sitol* |
| bio_355 | 0.262 | 0.0002694 | −0.052 | 0.028 | 0.702 | 0.000 | 0.996 | methylphosphate |
| bio_47 | 0.267 | 0.0001936 | 0.083 | −0.032 | 0.660 | −0.074 | 0.314 | 10-undecenoate (11:1n1) |
| bio_294 | 0.274 | 0.0001318 | 0.226 | 0.094 | 0.199 | −0.165 | 0.023 | histidine |
| bio_172 | 0.275 | 0.0001214 | 0.059 | 0.036 | 0.627 | −0.008 | 0.908 | benzoate |
| bio_261 | 0.279 | 0.0000965 | 0.197 | −0.168 | 0.021 | 0.186 | 0.010 | gamma-glutamyltyrosine |
| bio_262 | 0.281 | 0.0000854 | 0.201 | −0.181 | 0.013 | 0.153 | 0.035 | gamma-glutamylvaline |
| bio_552 | 0.293 | 0.0000399 | 0.133 | 0.087 | 0.238 | 0.171 | 0.019 | X-11793 |
| bio_565 | 0.311 | 0.000012 | 0.099 | 0.033 | 0.657 | 0.057 | 0.441 | X-11880 |
| bio_274 | 0.323 | 5.33E−06 | 0.142 | −0.019 | 0.800 | −0.012 | 0.871 | glycerol 2-phosphate |
| bio_529 | 0.326 | 4.25E−06 | 0.163 | 0.077 | 0.297 | 0.215 | 0.003 | X-11441 |
| bio_530 | 0.336 | 2.06E−06 | 0.175 | 0.065 | 0.377 | 0.262 | 0.000 | X-11442 |
| bio_109 | 0.349 | 7.25E−07 | 0.274 | −0.016 | 0.831 | 0.288 | 0.000 | 3-methyl-2-oxovalerate |
| bio_64 | 0.354 | 4.99E−07 | 0.311 | 0.048 | 0.511 | 0.057 | 0.441 | 2-hydroxybutyrate (AHB) |
| bio_523 | 0.373 | 9.53E−08 | 0.151 | 0.097 | 0.187 | −0.017 | 0.817 | X-11372 |
| bio_196 | 0.378 | 6.17E−08 | −0.014 | 0.089 | 0.225 | −0.020 | 0.783 | carnitine |
| bio_179 | 0.389 | 2.36E−08 | 0.176 | 0.185 | 0.011 | 0.144 | 0.048 | betaine |
| bio_501 | 0.400 | 8.13E−09 | 0.294 | −0.027 | 0.714 | 0.106 | 0.149 | valine |
| bio_495 | 0.409 | 3.35E−09 | 0.237 | −0.003 | 0.971 | 0.081 | 0.272 | tyrosine |
| bio_124 | 0.426 | 6.00E−10 | 0.314 | −0.002 | 0.981 | 0.240 | 0.001 | 4-methyl-2-oxopentanoate |
| bio_276 | 0.460 | 1.37E−11 | 0.272 | 0.029 | 0.697 | 0.069 | 0.350 | glycerophosphorylcholine (GPC) |
| bio_500 | 0.466 | 6.30E−12 | 0.250 | −0.049 | 0.503 | 0.107 | 0.144 | uridine |
| bio_482 | 0.474 | 2.33E−12 | 0.288 | −0.012 | 0.873 | 0.107 | 0.142 | threonine |
| bio_816 | 0.476 | 1.89E−12 | 0.192 | 0.010 | 0.893 | 0.143 | 0.050 | X-19380 |
| bio_817 | 0.528 | 1.33E−15 | 0.324 | −0.041 | 0.579 | 0.116 | 0.114 | X-19411 |
| bio_492 | 0.552 | 0 | 0.332 | −0.029 | 0.691 | 0.203 | 0.005 | tryptophan |
| bio_68 | | | | | | | | 2-hydroxyibuprofen |
| bio_697 | | | | | | | | X-16010 |
| bio_696 | | | | | | | | X-15824 |
| bio_443 | | | | | | | | quinine |
| bio_691 | | | | | | | | X-15707 |
| bio_212 | | | | | | | | cotinine N-oxide |
| bio_615 | | | | | | | | X-12741 |
| bio_437 | | | | | | | | pyridoxine (Vitamin B6) |
| bio_785 | | | | | | | | X-18307 |
| bio_685 | | | | | | | | X-15609 |
| bio_620 | | | | | | | | X-12805 |
| bio_683 | | | | | | | | X-15595 |
| bio_192 | | | | | | | | carbamazepine 10,11-epoxide* |
| bio_352 | | | | | | | | metformin |
| bio_194 | | | | | | | | carbamazepine* |
| bio_489 | | | | | | | | triamterene |
| bio_485 | | | | | | | | ticlopidine* |
| bio_419 | | | | | | | | phenylglyoxylic acid |
| bio_807 | | | | | | | | X-19124 |
| bio_445 | | | | | | | | ranitidine |
| bio_788 | | | | | | | | X-18485 |
| bio_310 | | | | | | | | ibuprofen acyl glucuronide |
| bio_358 | | | | | | | | mirtazapine |
| bio_395 | | | | | | | | omeprazole |
| bio_229 | | | | | | | | diphenhydramine |
| bio_203 | | | | | | | | cimetidine |
| bio_223 | | | | | | | | desvenlafaxine |
| bio_694 | | | | | | | | X-15731 |
| bio_754 | | | | | | | | X-17355 |
| bio_743 | | | | | | | | X-17188 |
| bio_564 | | | | | | | | X-11876 |
| bio_157 | | | | | | | | amitriptyline |
| bio_413 | | | | | | | | phenylalanylalanine |
| bio_646 | | | | | | | | X-13697 |
| bio_340 | | | | | | | | leucyltyrosine |
| bio_246 | | | | | | | | famotidine |
| bio_345 | | | | | | | | lipitor |
| bio_784 | | | | | | | | X-18275 |
| bio_245 | | | | | | | | ethyl glucuronide |
| bio_581 | | | | | | | | X-12179 |
| bio_672 | | | | | | | | X-14987 |
| bio_309 | | | | | | | | ibuprofen |
| bio_735 | | | | | | | | X-17161 |
| bio_235 | | | | | | | | doxylamine |
| bio_102 | | | | | | | | 3-hydroxyquinine |
| bio_193 | | | | | | | | carbamazepine glucuronide* |
| bio_638 | | | | | | | | X-13098 |
| bio_195 | | | | | | | | carboxyibuprofen |
| bio_421 | | | | | | | | phenylpropanolamine |

### REFERENCES

1. Kwong Y T, Stevens L A, Selvin E, et al. Imprecision of urinary iothalamate clearance as a gold-standard measure of GFR decreases the diagnostic accuracy of kidney function estimating equations. Am J Kidney Dis. July 2010; 56(1):39-49.
2. Levey A S, Bosch J P, Lewis J B, Greene T, Rogers N, Roth D. A more accurate method to estimate glomerular filtration rate from serum creatinine: a new prediction equation. Modification of Diet in Renal Disease Study Group. Ann Intern Med. Mar. 16 1999; 130(6):461-470.
3. Levey A S, Stevens L A, Schmid C H, et al. A new equation to estimate glomerular filtration rate. Ann Intern Med. May 5 2009; 150(9):604-612.
4. Kidney Disease: Improving Global Outcomes (KDIGO) CKD Work Group. KDIGO 2012 Clinical Practice Guideline for the Evaluation and Management of Chronic Kidney Disease. Kidney inter. 2013; Suppl. 3 1-150.
5. Inker L A, Schmid C H, Tighiouart H, et al. Estimating glomerular filtration rate from serum creatinine and cystatin C. The New England journal of medicine. Jul. 5 2012; 367(1):20-29.
6. Stevens L A, Coresh J, Schmid C H, et al. Estimating GFR using serum cystatin C alone and in combination with serum creatinine: a pooled analysis of 3,418 individuals with CKD. Am J Kidney Dis. March 2008; 51(3):395-406.
7. Miller W G, Myers G L, Ashwood E R, et al. Creatinine measurement: state of the art in accuracy and interlaboratory harmonization. Archives of pathology & laboratory medicine. March 2005; 129(3):297-304.
8. Israelit A H, Long D L, White M G, Hull A R. Measurement of glomerular filtration rate utilizing a single subcutaneous injection of 125I-iothalamate. Kidney Int. November 1973; 4(5):346-349.
9. Perrone R D. Steinman T I, Beck G J, et al. Utility of radioisotopic filtration markers in chronic renal insufficiency: simultaneous comparison of 125I-iothalamate, 169Yb-DTPA, 99mTc-DTPA, and inulin. The Modification of Diet in Renal Disease Study. Am J Kidney Dis. September 1990:16(3):224-235.
10. Levey A S, Coresh J, Greene T, et al. Using standardized serum creatinine values in the modification of diet in renal disease study equation for estimating glomerular filtration rate. Ann Intern Med. Aug. 15 2006; 145(4):247-254.
11. Stevens L A, Li S, Kurella Tamura M, et al. Comparison of the CKD Epidemiology Collaboration (CKD-EPI) and Modification of Diet in Renal Disease (MDRD) study equations: risk factors for and complications of CKD and mortality in the Kidney Early Evaluation Program (KEEP). Am J Kidney Dis. March 2011; 57(3 Suppl 2):S9-16.
12. Stevens L A, Manzi J, Levey A S, et al. Impact of creatinine calibration on performance of GFR estimating equations in a pooled individual patient database. Am J Kidney Dis. July 2007; 50(1):21-35.
13. Kyhse-Andersen J, Schmidt C, Nordin G, et al. Serum cystatin C, determined by a rapid, automated particle-enhanced turbidimetric method, is a better marker than serum creatinine for glomerular filtration rate. Clin Chem. October 1994; 40(10):1921-1926.
14. Evans A M. DeHaven C D, Barrett T. Mitchell M, Milgram E. Integrated, nontargeted ultrahigh performance liquid chromatography/electrospray ionization tandem mass spectrometry platform for the identification and relative quantification of the small-molecule complement of biological systems. Analytical chemistry. Aug. 15 2009; 81(16):6656-6667.
15. Ohta T. Masutomi N, Tsutsui N, et al. Untargeted metabolomic profiling as an evaluative tool of fenofibrate-induced toxicology in Fischer 344 male rats. Toxicologic pathology. June 2009; 37(4):521-535.
16. Yu B, Zheng Y, Alexander D, Morrison A C, Coresh J, Boerwinkle E. Genetic determinants influencing human serum metabolome among African Americans. PLoS genetics. March 2014; 10(3):e1004212.
17. Nkuipou-Kenfack E, Duranton F, Gayrard N, et al. Assessment of metabolomic and proteomic biomarkers in detection and prognosis of progression of renal function in chronic kidney disease. PLoS One. 2014; 9(5):e96955.
18. Goek O N, Doring A, Gieger C, et al. Serum metabolite concentrations and decreased GFR in the general population. Am J Kidney Dis. August 2012; 60(2):197-206.
19. Buzatto A Z, de Sousa A C, Guedes S F. Cieslarova Z, Simionato A V. Metabolomic investigation of human diseases biomarkers by CE and LC coupled to MS. Electrophoresis. May 2014; 35(9): 1285-1307.
20. Kobayashi T, Yoshida T, Fujisawa T, et al. A metabolomics-based approach for predicting stages of chronic kidney disease. Biochemical and biophysical research communications. Mar. 7 2014; 445(2):412-416.
21. Rhee E P, Clish C B, Ghorbani A, et al. A combined epidemiologic and metabolomic approach improves CKD prediction. J Am Soc Nephrol. July 2013; 24(8):1330-1338.
22. Mullen W, Saigusa D, Abe T, Adamski J, Mischak H. Proteomics and metabolomics as tools to unravel novel culprits and mechanisms of uremic toxicity: instrument or hype? Seminars in nephrology. March 2014; 34(2): 180-190.
23. Niewczas M A, Sirich T L, Mathew A V, et al. Uremic solutes and risk of end-stage renal disease in type 2 diabetes: metabolomic study. Kidney Int. May 2014; 85(5):1214-1224.
24. Zheng Y, Yu B, Alexander D, Steffen L M, Boerwinkle E. Human metabolome associates with dietary intake habits among african americans in the atherosclerosis risk in communities study. American journal of epidemiology. Jun. 15 2014; 179(12): 1424-1433.
25. Miller W G. Reporting estimated GFR: a laboratory perspective. Am J Kidney Dis. October 2008; 52(4):645-648.
26. Myers G L, Miller W G, Coresh J, et al. Recommendations for improving serum creatinine measurement: a report from the Laboratory Working Group of the National Kidney Disease Education Program. Clin Chem. January 2006; 52(1):5-18.
27. Stevens L A, Levey A S. Measured GFR as a confirmatory test for estimated GFR. J Am Soc Nephrol. November 2009; 20(11):2305-2313.

1. A method for calculating the estimated glomerular filtration rate (eGFR) in a patient comprising the steps of:
a. measuring the level of one or more metabolites using mass spectrometry from a blood sample obtained from the patient; and
b. calculating the eGFR using an algorithm that utilizes the measured levels of the one or more metabolites, wherein the algorithm is developed using GFR measured using an exogenous filtration marker.

2. The method of claim 1, wherein the one or more metabolites comprise one or more of X-11564, C-glycosyltryptophan, p-cresol sulfate, myo-inositol, X-02249, and pseudouridine.

3. The method of claim 1, wherein the one or more metabolites comprise one or more of creatinine, X-11564, C-glycosyltryptophan, 1-methylhistidine, leucine, and 1-myristoylglycerophosphocholine (14:0).

4. The method of claim 1, wherein the one or more metabolites comprise one or more of C-glycosyltryptophan, myo-inositol, pseudouridine, N-acetyl-1-methylhistidine, and phenylacetylglutamine.

5. The method of claim 1, wherein the one or more metabolites comprise one or more of creatinine, C-glycosyltryptophan, pseudouridine, myo-inositol, and phenylacetylglutamine.

6. The method of claim 1, wherein the one or more metabolites comprise one or more of X-11564, C-glycosyltryptophan, pseudouridine, X-17299, N-acetylthreonine, N-acetylserine, erythritol, arabitol, urea, and X-16394.

7. The method of claim 1, wherein the one or more metabolites comprise one or more of X-11564, C-glycosyltryptophan, pseudouridine, X-17299, and N-acetylthreonine.

8. The method of claim 1, wherein the one or more metabolites comprise one or more of C-glysyltryptophan*, pseudouridine, N-acetyl-threonine, N-acetylserine, and erythritol.

9. The method of claim 1, wherein the one or more metabolites comprise one or more of valine, tyrosine, 4-methyl-2-oxopentanoate, glycerophosphorylcholine (GPC), uridine, threonine, X-19380, X-19411, tryptophan, X-11564, C-glycosyltryptophan*, pseudouridine, X-17299, N-acetylthreonine, N-acetylserine, erythritol, arabitol, urea, X-16394, X-11423, erythronate*, creatinine, myo-inositol, N6-carbamoylthreonyladenosine, X-12749, X-12104, N-acetylalanine, N2,N2-dimethylguanosine, 4-acetamidobutanoate, X-11945, 1-methylhistidine, arabonate, N-formylmethionine, 2-hydroxyisobutyrate, xylonate, succinylcarnitine, N-acetylneuraminate, X-12686, N-acetyl-1-methylhistidine*, homocitrulline, X-17703, X-11444, threitol, X-18887, X-12846, p-cresol sulfate, 3-methylglutarylcarnitine (C6), N1-Methyl-2-pyridone-5-carboxamide, glutarylcarnitine (C5), X-16982, isobutyrylcarnitine, 3-indoxyl sulfate, X-17357, galactitol (dulcitol), X-12822, X-13837, X-02249, X-12411, X-13844, kynurenine, X-12007, X-13553, X-12125, N2,N5-diacetylornithine, O-methylcatechol sulfate, X-13835, X-12729, X-12814, leucine, and 1-myristoylglycerophosphocholine (14:0), betaine, 2-hydroxybutyrate (AHB), X-18914.

10. The method of claim 1, wherein the algorithm further utilizes serum creatinine levels.

11. The method of claim 1, wherein the algorithm further utilizes serum cystatin C levels.

12. The method of claim 1, wherein the algorithm further utilizes one or more demographic parameters selected from the group consisting of age, sex and race.

13. The method of claim 1, wherein the algorithm further utilizes one or more of serum creatinine levels, serum cystatin C levels, age, sex and race.

14. The method of claim 1, wherein the algorithm is a linear model.

15. The method of claim 1, wherein the algorithm is a non-linear model.

16. A method for calculating the estimated GFR in a patient comprising the steps of:
a. measuring the level of one or more metabolites using mass spectrometry from a blood sample obtained from the patient, wherein the one or more metabolites comprise X-11564, C-glycosyltryptophan, pseudouridine, X-17299, and N-acetylthreonine; and
b. calculating the estimated GFR using an algorithm that utilizes the measured levels of the metabolites and one or more of serum creatinine levels, serum cystatin C levels, age, sex and race.

17. A method for calculating the estimated GFR in a patient comprising the steps of:
c. measuring the level of one or more metabolites from a blood sample obtained from the patient, wherein the one or more metabolites comprise X-11564, C-glycosyltryptophan, pseudouridine, X-17299, and N-acetylthreonine; and
d. calculating the estimated GFR using an algorithm that utilizes the measured levels of the metabolites and one or more of serum creatinine levels, serum cystatin C levels, age, sex and race.

18. The method of claim 17, wherein the measuring step is performed using mass spectrometry.

19. A method for determining the estimated GFR in a patient comprising the step of calculating the estimated GFR using an algorithm that utilizes the measured levels of one or more metabolite biomarkers and one or more of serum creatinine levels, serum cystatin C levels, age, sex and race, wherein the metabolite biomarkers comprise X-11564, C-glycosyltryptophan, pseudouridine, X-17299, and N-acetylthreonine, and further wherein the metabolite biomarkers are measured from a blood sample obtained from the patient.

20. The method of claim 16, wherein the algorithm is a linear model.

21. The method of claim 16, wherein the algorithm is a non-linear model.

22. The method of claim 1, wherein the algorithm is a stepwise regression model.
